(19) United States
(12) Patent Application Publication
(10) Pub. No.: US 2021/0163969 A1
(43) Pub. Date: Jun. 3, 2021

(54) COMBINED TRANSCRIPTION AND TRANSLATION PLATFORM DERIVED FROM PLANT PLASTIDS AND METHODS FOR IN VITRO PROTEIN SYNTHESIS AND PROTOTYPING OF GENETIC EXPRESSION IN PLANTS

(71) Applicants: Northwestern University, Evanston, IL (US); Massachusetts Institute of Technology, Cambridge, MA (US)

(72) Inventors: Michael Christopher Jewett, Evanston, IL (US); Lauren Clark, Chicago, IL (US); Christopher A. Voigt, Cambridge, MA (US); Eszter Majer, Boston, MA (US)

(21) Appl. No.: 17/170,702

(22) Filed: Feb. 8, 2021

Related U.S. Application Data
(63) Continuation-in-part of application No. PCT/US2019/045319, filed on Aug. 6, 2019.
(60) Provisional application No. 62/772,341, filed on Nov. 28, 2018.
(60) Provisional application No. 62/714,916, filed on Aug. 6, 2018.
(60) Provisional application No. 62/976,316, filed on Feb. 13, 2020.

(51) Int. Cl. C12N 15/82; C12N 15/10

(52) U.S. Cl. CPC C12N 15/8214; C12N 15/1086; C12N 15/8251

(57) ABSTRACT

Disclosed are compositions, methods, and kits for performing cell-free protein synthesis (CFPS). The disclosed compositions, methods, and kits include or utilize components prepared from plant plastids or extracts thereof. The compositions, methods, and kits may be used for in vitro protein synthesis and prototyping of genetic expression in plants and are suitable for automation.

### CROSS-REFERENCE TO RELATED PATENT APPLICATIONS

The present application is a continuation-in-part (CIP) application of International Application PCT/US2019/045319, filed on Aug. 6, 2019, which claims the benefit of priority under 35 U.S.C. § 119(e) to U.S. Provisional Application No. 62/772,341, filed on Nov. 28, 2018, and to U.S. Provisional Application No. 62/714,916, filed on Aug. 6, 2018. The present application also claims the benefit of priority to U.S. Provisional Application No. 62/976,316, filed on Feb. 13, 2020. The contents of the aforementioned applications are incorporated herein by reference in their entireties.

### STATEMENT REGARDING FEDERALLY SPONSORED RESEARCH OR DEVELOPMENT

This invention was made with government support under HR0011-15-C-0084 awarded by the Defense Advanced Research Projects Agency (DARPA). The government has certain rights in the invention.

### BACKGROUND

The present invention generally relates to compositions, methods, and kits for performing cell-free protein synthesis (CFPS). More specifically, the present invention relates to compositions, methods, and kits for performing cell-free protein synthesis (CFPS) that include or utilize components prepared from isolated plant plastids or extracts thereof. The compositions, methods, and kits may be used for in vitro protein synthesis and prototyping of genetic expression in plants.

Cell-free systems have recently enjoyed a technical renaissance that has transformed them into robust platforms for the synthesis of a wide variety of useful and interesting products[1-4]. Such platforms combine crude cell lysates or purified components with substrates in a test tube, enabling the activation and use of cellular processes in vitro. Cell-free protein synthesis (CFPS) systems in particular have made significant advances in reaction volume, duration, and productivity, now reaching g/L quantities of protein[3, 5-12]. These systems provide several unique advantages for understanding, harnessing, and expanding the capabilities of natural biological systems. Reactions are open, and are therefore easily accessible for sample extraction and substrate feeding. Dilute reaction environments facilitate the folding of complex eukaryotic protein products which may otherwise express poorly in bacterial systems[4]. Importantly, the removal of genomic material from the chassis organism directs reaction substrates and machinery towards the desired synthesis reaction at high rates. Exploiting these features, CFPS platforms enjoy increasingly widespread use as a complement to in vivo expression for applications including biomolecular breadboarding[13-16], expression of toxic products[17-20], production of complex protein products that are poorly soluble in vivo[7, 21-23], manufacture of glycoproteins[24, 25], detection of disease[26-28], and on demand biomanufacturing[21, 29-32].

Despite the emergence of cell-free systems as a prominent research tool for fundamental and applied biology, the vast majority of previous efforts have focused on a select few model systems such as Escherichia coli, Saccharomyces cerevisiae, and Chinese Hamster Ovary cells, among others[2, 5, 11, 12, 33, 34]. However, we and others hypothesize that developing cell-free systems composed of extracts derived from relevant chassis organisms that better mimic the natural physicochemical environment might enhance predictive power for synthetic biology applications. This idea motivates the development of new cell-free systems. In this context, several new CFPS systems have been developed, including some from Streptomyces species and Bacillus [35-39]. For example, an elegant study by Freemont and colleagues showed characterized new DNA parts from the non-model bacterium Bacillus megaterium by combining automated CFPS and Bayesian models[39].

A particularly exciting chassis organism for developing a new cell-free system is plant plastids, and plant chloroplasts in particular. Unfortunately, the current state of the art in transformation of plants is a laborious, low-throughput method that takes approximately one year to produce a stably transgenic plant. As a result, developments in plant biotechnology and our understanding of the basic biology of plants have lagged far behind what we have been able to achieve with mammalian or prokaryotic biology, in spite of the fact that plants are considered to be the source of a rich potential for pharmaceutically and technologically relevant natural products. Developing a cell-free system that could be used to prototype genetic design and establish part libraries for plants could be transformative. Indeed, new genetic parts (e.g., promoters, ribosome binding sites, terminators) could facilitate forward engineering.

Here, we disclose a cell-free system using components prepared from plant plastids and extracts thereof using Nicotiana tabacum and Zea mays as models. Our disclosed cell-free systems can be applied to prepare similar cell-systems using components prepared from other plants in order to prototype genetic parts and establish part libraries.

### SUMMARY

Disclosed are components, compositions, systems, kits, and methods for performing cell-free protein synthesis (CFPS). The disclosed components may be prepared from isolated plant plastids and extracts thereof, and the disclosed compositions, systems, kits, and methods may include or utilize components prepared from isolated plant plastids and extracts thereof. In particular, the disclosed components may be prepared from isolated chloroplasts and extracts thereof, and the disclosed compositions, kits, and methods may include or utilize components prepared from isolated plant chloroplasts and extracts thereof. The components, compositions, systems, kits, and methods may be used for in vitro protein synthesis and prototyping of genetic expression in plants. The systems and methods disclosed herein are suitable for automation.

The present disclosure is based, at least in part, on the discovery of a plastid cell-free protein synthesis system that enables high throughput genetic analysis and protein production. In some aspects, a plant plastid cell-free protein synthesis system for in vitro transcription of mRNA and/or translation of polypeptides is provided.

The disclosed components, compositions, systems, kits, and methods relate to plants and plant materials, such as extracts prepared from isolated plastid of plants, such as extracts prepared from isolated chloroplasts. Suitable plants for use in preparing the disclosed components, compositions, systems, and kits disclosed herein may include, but are not limited to tobacco (e.g., Nicotiana spp. such as Nicotiana tabacum) and maize (e.g., Zea spp. such as Zea mays L.) For example, suitable plants for preparing the disclosed plastid extracts may include, but are not limited to tobacco chloroplasts and maize chloroplasts.

Particularly disclosed herein are cell-free protein synthesis systems that comprise as a component a plastid extract prepared from plant tissue or cells, optionally wherein the extract is not prepared from whole leaf (i.e., wherein the plastids are isolated from whole leaf prior to preparing the extract). In some embodiments, the plastid extract may be prepared from plant tissue or cells, optionally wherein the plant has been grown for 1-, 2-, 3-, 4-, 5-, 6-, or 7-8 weeks (i.e., post-planting or post-germination) prior to processing the plant to obtain the plastid extract. Optionally, the cell-free protein synthesis systems further may comprise one or more components selected from a reaction buffer; an RNA polymerase; and a transcription template, wherein the RNA polymerase is capable of transcribing the transcription template to form a translation template and the plastid extract can sustain protein synthesis through a combined transcription/translation reaction.

In some embodiments, the plant utilized for preparing the components, compositions, systems, and kits disclosed herein has been grown for about 2 weeks (e.g., post-planting or post-germination). In some embodiments, the plant has been grown in about 8 hours of light and about 16 hours of darkness. In some embodiments, the plant has been grown in about 10 hours of darkness immediately before the extract is prepared.

In some embodiments, the plastid extract is prepared from a plant that has been engineered to express T7 RNA polymerase in the plastid or in the nucleus. In some embodiments, the plastid extract is prepared from a plant that has been engineered to express T7 RNA polymerase in the nucleus and the T7 RNA polymerases comprises a polypeptide signal that targets the T7 RNA polymerase to the plastid.

In some embodiments, the plant is engineered to be deficient in a negative effector for cell-free protein synthesis (CFPS). For example, the plant may engineered to be deficient in a negative effector for transcription and/or translation.

Also disclosed herein are components, compositions, systems, kits, and methods for performing in vitro transcription of mRNA and/or in vitro translation of a polypeptide. The methods may involve reacting a plant plastid cell-free protein synthesis mixture or system with a linear or plasmid template for transcription of the mRNA and/or a template for translation of the polypeptide. Optionally, the cell-free protein synthesis system comprises one or more of monomers for synthesis of the mRNA and/or the polypeptide; co-factors, enzymes and/or other reagents necessary for the transcription and/or translation; and magnesium at a concentration of from about 3 mM to about 20 mM (e.g., from about 4 mM to about 12 mM, or from about 6 mM to about 10 mM).

In some embodiments, the disclosed compositions, systems, kits, and methods may include or utilize monomers. For example, the compositions, systems, kits, and methods may include nucleotide monomers for synthesizing mRNA and/or monomers for synthesizing polypeptides. In some embodiments, the monomers are amino acids present in the disclosed compositions, systems, kits, and methods at a concentration of between about 0.5 to 4 mM, and preferably at a concentration of about 2 mM. In some embodiments, the disclosed compositions, systems, kits, and methods may include or utilize potassium at a concentration of about 0-, 1-, 2-, 3-, 4-, or 5-200 mM, and preferably at a concentration of about 100 mM. In some embodiments, the disclosed compositions, systems, kits, and methods may include or utilize salts at a total concentration of between about 50-400 mM. In some embodiments, the systems or methods are actuated or performed using a liquid handling robot.

In some embodiments, the template encodes a test library of genetic components of a plant (e.g., tobacco or maize). In some embodiments, the method further comprises testing the function of the genetic components of the test library in the plant plastid cell-free protein synthesis system and assessing gene expression in the system. In some embodiments, the methods are used to characterize and assess the genetic parts or components and modulate gene expression prior to studying the genetic components and gene expression in plants. In some embodiments, the genetic components comprise at least two genetic components selected from the group consisting of promoters, terminators, ribosome binding sites, and genes. In some embodiments, the genetic components are genetic clusters.

In some embodiments, the template encodes a test library of codon-optimized constructs of a gene product. In some embodiments, the method further comprises testing expression of the codon-optimized constructs of the test library in the plant plastid cell-free protein synthesis system and using information obtained from testing expression of the codon-optimized constructs to modify expression in a plant. In some embodiments, the method may be used to characterize and assess codon optimization and modulate of gene expression prior to studying codon optimization and modulate of gene expression in plants.

In some embodiments, the template encodes a test library of constructs expressing several genes. In some embodiments, the method further comprises testing expression of the constructs of the test library in the plant plastid cell-free protein synthesis system and using information obtained from testing expression of the construct to modify gene expression in a plant. In some embodiments, the method may be used to provide information to a geneticist regarding the design of multi gene functions.

In some embodiments, the template encodes a test library of constructs of a biosynthetic pathway expressing enzymes. In some embodiments the method further comprises testing the constructs of the test library in the plant plastid cell-free protein synthesis system and assessing gene expression, metabolite production, requirement of accessory proteins or other molecules, and/or metabolic parameters. In some embodiments, the method may be used to aid design of multi gene functions, and the method may be used to aid the design and function of enzyme pathways for example in order to modulate expression levels of the enzymes and testing possible accessory proteins or other molecules required for proper pathway function.

In some embodiments, the template encodes plant specific sensors comprising genetic circuits that respond to external commands. In some embodiments, the method further comprises testing the sensors in the plant plastid cell-free protein synthesis system and assessing sensor response and circuit behavior. In some embodiments, the method may be used to aid the design and function of sensor and genetic circuits.

### BRIEF DESCRIPTION OF THE FIGURES

The following drawings form part of the present specification and are included to further demonstrate certain aspects of the present disclosure, which can be better understood by reference to one or more of these drawings in combination with the detailed description of specific embodiments presented herein. For purposes of clarity, not every component may be labeled in every drawing. It is to be understood that the data illustrated in the drawings in no way limit the scope of the disclosure.

FIG. 1 provides an overview of the isolation of intact chloroplasts from plant material and preparation of an extract from the isolated chloroplasts for use in cell-free transcription and/or translation.

FIG. 2 illustrates the relative protein concentration of ribulose-1,5-bisphosphate carboxylase/oxygenase (Rubisco), an enzyme present in chloroplasts, versus the concentration of membrane proteins in a extract as an indicator of intact chloroplasts versus broken chloroplasts.

FIG. 3 illustrates the production of mRNA in a cell-free system comprising a chloroplast extract as a function of the concentration of a DNA template for mRNA present in the system.

FIG. 4 illustrates the results of experiments in which a template DNA encoding a fluorescent target protein was transcribed and translated in a cell-free systems comprising a chloroplast extract.

FIG. 5 illustrates that divalent cations such as Mg²⁺ are required for efficient protein production in a cell-free system comprising a chloroplast extract in which a template DNA encoding a fluorescent target protein was transcribed and translated as in FIG. 4.

FIG. 6 illustrates that extracts prepared from chloroplasts isolated from plants that were grown at six (6) weeks versus four (4) weeks exhibit better performance regarding protein production in a cell-free free system.

FIG. 7 illustrates a method to increase lysate activity related to total protein content by e.g. dark incubation of plants before extract preparation. a) Light conditions during normal growth and dark incubation of plants before chloroplast extract purification. After 6-24 h of dark incubation starch granules are reduced in the chloroplasts (light microscopic images) which results in b) higher total protein content (measured with Bradford assay) and lysate productivity (measured as expressed luciferase) in the chloroplast cell-free system. SD from technical replicates of purified lysates.

FIG. 8 illustrates the results of semi-continuous and batch reaction of extracts from light/dark incubated plants (2 replicates: performed with two different extracts).

FIG. 9 illustrates that the addition of a polyol such as glycerol to isolated chloroplasts prior to lysis by freezing improves productivity of the lysates.

FIG. 10 illustrates the results of combined optimization on protein production in a cell-free system comprising a chloroplast extract, including addition of a polyol prior to lysis of chloroplasts to prepare the extract, timing of growth in plants from which the chloroplast extract is prepared, and codon optimization for expression in the plants from which the chloroplast extract is prepared.

FIG. 11 illustrates that protein expression is influenced by modification in the 5′-UTR and 3′-UTR of the expression template.

FIG. 12 illustrates the effect of magnesium acetate versus magnesium glutamate on protein production in a cell-free system comprising a chloroplast extract.

FIG. 13 illustrates the effect of adding potassium on protein production in a cell-free system comprising a chloroplast extract.

FIG. 14 illustrates the effect of pH in the presence of a HEPES buffered system versus a Tris buffered system on protein production in a cell-free system comprising a chloroplast extract.

FIG. 15 illustrates the effect of adding ammonium on protein production in a cell-free system comprising a chloroplast extract.

FIG. 16 illustrates the effect of amino acid concentration on protein production in a cell-free system comprising a chloroplast extract.

FIG. 17 illustrates the effect of HEPES concentration at a pH of 7.3 on protein production in a cell-free system comprising a chloroplast extract.

FIG. 18 illustrates the effect of different energy regeneration systems on protein production in a cell-free system comprising a chloroplast extract, including a phosphoenolpyruvate (PEP) system, a PEP/pyruvate kinase (PyK) system, and a creatine phosphate (CP)/creatine kinase (CK) system.

FIG. 19 illustrates the effect of different concentrations of a creatine phosphate (CP)/creatine kinase (CK) energy regeneration system on protein production in a cell-free system comprising a chloroplast extract.

FIG. 20 illustrates the effect of different concentration of T7 DNA-dependent RNA-polymerase (T7 RNAP) on protein production in a cell-free system comprising a chloroplast extract.

FIG. 21 illustrates the effect of final DNA template concentration at two different concentrations of T7 RNAP on protein production in a cell-free system comprising a chloroplast extract.

FIG. 22 illustrates the effect of different macromolecular crowding agents on protein production in a cell-free system comprising a chloroplast extract, including Ficoll 400, PEG 600, PEG 3350, and PEG 8000.

FIG. 23 illustrates the effect of different macromolecular crowding agents (Ficoll 400, PEG 3350, and PEG 8000) at different concentrations (0.50%, 1%, 2%, and 4%) on protein production in a cell-free system comprising a chloroplast extract.

FIG. 24 illustrates the effect of chloroplast extract concentration on protein production in a cell-free system comprising the chloroplast extract.

FIG. 25 illustrates protein production versus time in two different cell-free systems comprising a chloroplast extract.

FIG. 26 shows the effect of RNAse inhibitor on chloroplast cell-free reaction. 0, 0.5 and 1 U/μL RNAse inhibitor was added.

FIG. 27 illustrates that linear templates or plasmid DNA can be used in chloroplast cell free reactions to produce protein, with linear templates offering a slight boost to yields and a faster turnaround time than plasmids.

FIG. 28 illustrates different mRNA expression dynamics in chloroplast and E. coli cell-free systems. A construct (pY71mRFP1SpA) expressing a fluorescent protein (mRFP) and a fluorescent RNA aptamer (spinach) was assayed in both chloroplast and E. coli cell-free systems.

FIG. 29 provides a schematic illustration of plastid prototyping using a chloroplast cell-free system and subsequent chloroplast transformation to prepare modified plants.

FIG. 30 illustrates a thermodynamic model that provides the basis for designing ribosome binding sites (RBS) that have different strengths.

FIG. 31 illustrates the results of experiments in which protein expression was measured versus ribosome binding strength for a library of expression vectors having different ribosome binding sites (RBS). The proteins expressed in the system included the first 99 nucleotides of biosynthetic enzyme A (Kas) or biosynthetic enzyme B (AroG) fused to luciferase where luciferase activity was measures as an output for protein production.

FIG. 32 illustrates the results of experiments in which protein expression was measured versus ribosome binding strength for a library of expression vectors having different ribosome binding sites (RBS). The proteins expressed in the system were one of biosynthetic enzyme A (Kas) or biosynthetic enzyme B (AroG) where protein production was measure via incorporation of radioactive Leucine.

FIG. 33 illustrates yields of protein (GFP) in a chloroplast extract using an RBS library and a 16S rRNA sequence from the chloroplast, illustrating how a chloroplast cell-free system can be utilized to rank the efficiency of genetic parts such as RBS.

FIG. 34 illustrates schematically how the effect of codon-optimization on protein production can be assessed using a chloroplast cell-free system and how an algorithm can be devised from results in a chloroplast cell-free system to provide an algorithm for codon optimization prior to chloroplast transformation of an expression vector.

FIG. 35 illustrates RT-qPCR analysis of a multi-genic cluster in a chloroplast cell-free system. A cluster (Klebsiella‘refactored’ v2.) composed of 16 genes (20 kb) was used as template for chloroplast cell-free reactions. Reactions were incubated 25° C. during 1 h and 100 μL of the reaction mix was harvested for total RNA extraction. Total RNA was assayed with RT-qPCR. All 16 genes were detected in both cell-free systems, demonstrating that the chloroplast cell-free is able to express a multi-gene cluster.

FIG. 36 provides a comparison of multi-gene expression in a cell-free system per FIG. 37 versus multi-gene expression in vivo.

FIG. 37 illustrates RT-qPCR analysis of a multi-genic cluster in chloroplast and E. coli cell-free. a) A cluster (Klebsiella ‘refactored’ v2.) composed of 16 genes (20 kb) was used as template for chloroplast and E. coli cell-free reactions. Reactions were incubated 25° C. or 37° C. during 1 h and 100 μL of the reaction mix was harvested for total RNA extraction. b) Total RNA was assayed with RT-qPCR. All 16 genes were detected in both cell-free systems, demonstrating that the chloroplast cell-free is able to express a multi-gene cluster.

FIG. 38 illustrates that there is a better correlation between predicted RBS strength and actual yields in a cell-free system that comprises a chloroplast extract versus a cell-free system that comprise an E. coli extract.

FIG. 39 Chloroplast in vivo gene expression correlates with chloroplast cell-free data. a) Engineered nitrogenase (nif) clusters (Klebsiella v1.0, v2.0, and v3.2) were integrated into the tobacco plastome under T7 promoters. Upon theophylline (Theo) induction, plastome integrated T7 RNAP transcribes nif clusters. b) Plasmids with nif clusters assayed in the chloroplast cell-free system. GFP under T7 promoter was expressed from the plasmid backbone for normalization. Purified T7 RNAP was added to the reactions. c) RNA-seq data of transplastomic plants (left hand side) shows cluster expression with and without Theo induction (full and empty circles, respectively). Correlation between wild type Klebsiella oxytoca and transplastomic lines (right hand side). d) RT-qPCR data normalized to GFP shows cluster expression in chloroplast cell-free (left hand side). Correlation between in vivo and chloroplast cell-free data (right hand side).

FIG. 40 illustrates the preparation of extracts from isolated intact chloroplasts (cp) obtained from lysed maize cells for in vitro transcription and in vitro translation of a template encoding luciferase. Intact chloroplasts (cp) were isolated from other cellular components including broken chloroplasts and thylakoids (tk).

FIG. 41(a), FIG. 41(b), and FIG. 41(c) illustrate purification of the maize cell-free system.

FIG. 42 demonstrates that the maize cell-free system synthesizes protein from a DNA template.

FIG. 43(a), FIG. 43(b), and FIG. 43(c) demonstrate that the maize cell-free system transcribes a multi-genic construct.

### DETAILED DESCRIPTION

### Definitions and Terminology

The disclosed subject matter may be further described using definitions and terminology as follows. The definitions and terminology used herein are for the purpose of describing particular embodiments only, and are not intended to be limiting.

As used in this specification and the claims, the singular forms “a,” “an,” and “the” include plural forms unless the context clearly dictates otherwise. For example, the term “a component” should be interpreted to mean “one or more components” unless the context clearly dictates otherwise. As used herein, the term “plurality” means “two or more.”

As used herein, “about”, “approximately,” “substantially,” and “significantly” will be understood by persons of ordinary skill in the art and will vary to some extent on the context in which they are used. If there are uses of the term which are not clear to persons of ordinary skill in the art given the context in which it is used, “about” and “approximately” will mean up to plus or minus 10% of the particular term and “substantially” and “significantly” will mean more than plus or minus 10% of the particular term.

As used herein, the terms “include” and “including” have the same meaning as the terms “comprise” and “comprising.” The terms “comprise” and “comprising” should be interpreted as being “open” transitional terms that permit the inclusion of additional components further to those components recited in the claims. The terms “consist” and “consisting of” should be interpreted as being “closed” transitional terms that do not permit the inclusion of additional components other than the components recited in the claims. The term “consisting essentially of” should be interpreted to be partially closed and allowing the inclusion only of additional components that do not fundamentally alter the nature of the claimed subject matter.

The phrase “such as” should be interpreted as “for example, including.” Moreover the use of any and all exemplary language, including but not limited to “such as”, is intended merely to better illuminate the invention and does not pose a limitation on the scope of the invention unless otherwise claimed.

Furthermore, in those instances where a convention analogous to “at least one of A, B and C, etc.” is used, in general such a construction is intended in the sense of one having ordinary skill in the art would understand the convention (e.g., “a system having at least one of A, B and C” would include but not be limited to systems that have A alone, B alone, C alone, A and B together, A and C together, B and C together, and/or A, B, and C together.). It will be further understood by those within the art that virtually any disjunctive word and/or phrase presenting two or more alternative terms, whether in the description or figures, should be understood to contemplate the possibilities of including one of the terms, either of the terms, or both terms. For example, the phrase “A or B” will be understood to include the possibilities of “A” or 'B or “A and B.”

All language such as “up to,” “at least,” “greater than,” “less than,” and the like, include the number recited and refer to ranges which can subsequently be broken down into subranges as discussed above.

A range includes each individual member. Thus, for example, a group having 1-3 members refers to groups having 1, 2, or 3 members. Similarly, a group having 6 members refers to groups having 1, 2, 3, 4, or 6 members, and so forth.

The modal verb “may” refers to the preferred use or selection of one or more options or choices among the several described embodiments or features contained within the same. Where no options or choices are disclosed regarding a particular embodiment or feature contained in the same, the modal verb “may” refers to an affirmative act regarding how to make or use and aspect of a described embodiment or feature contained in the same, or a definitive decision to use a specific skill regarding a described embodiment or feature contained in the same. In this latter context, the modal verb “may” has the same meaning and connotation as the auxiliary verb “can.”

The term “plastid” refers to a small organelle of plant cells which may include pigment (e.g., chlorophyll) or food (e.g., starch). Plastids may include, but are not limited to (chloroplasts, amyloplasts, elaioplasts, chromoplasts, etc.) Plant plastids (chloroplasts, amyloplasts, elaioplasts, chromoplasts, etc.) are the major biosynthetic centers that in addition to photosynthesis are responsible for production of industrially important compounds such as amino acids, complex carbohydrates, fatty acids, and pigments. Plastids are derived from a common precursor known as a proplastid and thus the plastids present in a given plant species all have the same genetic content. Plant cells contain 500-10,000 copies of a small 120-160 kilobase circular genome, each molecule of which has a large (approximately 25 kb) inverted repeat. Thus, it is possible to engineer plant cells to contain up to 20,000 copies of a particular gene of interest, which potentially can result in very high levels of foreign gene expression.

As used herein, the term “modulate” refers to altering a condition or result from a reference level, such as a wild-type level. For example, modulating expression of gene may refer to increasing expression of a gene or decreasing expression of a gene from a reference level, such as a wild-type level.

Polynucleotides and Synthesis Methods

The terms “nucleic acid” and “oligonucleotide,” as used herein, refer to polydeoxyribonucleotides (containing 2-deoxy-D-ribose), polyribonucleotides (containing D-ribose), and to any other type of polynucleotide that is an N glycoside of a purine or pyrimidine base. There is no intended distinction in length between the terms “nucleic acid”, “oligonucleotide” and “polynucleotide”, and these terms will be used interchangeably. These terms refer only to the primary structure of the molecule. Thus, these terms include double- and single-stranded DNA, as well as double- and single-stranded RNA. For use in the present methods, an oligonucleotide also can comprise nucleotide analogs in which the base, sugar, or phosphate backbone is modified as well as non-purine or non-pyrimidine nucleotide analogs.

As used herein, a “polymerase” refers to an enzyme that catalyzes the polymerization of nucleotides. “DNA polymerase” catalyzes the polymerization of deoxyribonucleotides. Known DNA polymerases include, for example, Pyrococcus furiosus (Pfu) DNA polymerase, E. coli DNA polymerase I, T7 DNA polymerase and Thermus aquaticus (Taq) DNA polymerase, among others. “RNA polymerase” catalyzes the polymerization of ribonucleotides. The foregoing examples of DNA polymerases are also known as DNA-dependent DNA polymerases. RNA-dependent DNA polymerases also fall within the scope of DNA polymerases. Reverse transcriptase, which includes viral polymerases encoded by retroviruses, is an example of an RNA-dependent DNA polymerase. Known examples of RNA polymerase (“RNAP”) include, for example, RNA polymerases of bacteriophages (e.g. T3 RNA polymerase, T7 RNA polymerase, SP6 RNA polymerase), and E. coli RNA polymerase, among others. The foregoing examples of RNA polymerases are also known as DNA-dependent RNA polymerase. The polymerase activity of any of the above enzymes can be determined by means well known in the art.

The term “promoter” refers to a cis-acting DNA sequence that directs RNA polymerase and other trans-acting transcription factors to initiate RNA transcription from the DNA template that includes the cis-acting DNA sequence.

As used herein, the term “sequence defined biopolymer” refers to a biopolymer having a specific primary sequence. A sequence defined biopolymer can be equivalent to a genetically-encoded defined biopolymer in cases where a gene encodes the biopolymer having a specific primary sequence.

As used herein, “expression template” refers to a nucleic acid that serves as substrate for transcribing at least one RNA that can be translated into a sequence defined biopolymer (e.g., a polypeptide or protein). Expression templates include nucleic acids composed of DNA or RNA. Suitable sources of DNA for use a nucleic acid for an expression template include genomic DNA, cDNA and RNA that can be converted into cDNA. Genomic DNA, cDNA and RNA can be from any biological source, such as a tissue sample, a biopsy, a swab, sputum, a blood sample, a fecal sample, a urine sample, a scraping, among others. The genomic DNA, cDNA and RNA can be from host cell or virus origins and from any species, including extant and extinct organisms. As used herein, “expression template” and “transcription template” have the same meaning and are used interchangeably.

In some embodiments of the present disclosure the expression template may be a genetic cluster. A genetic cluster includes a nucleotide sequence that is at least about 85% or more homologous or identical relative to the entire length of a naturally occurring genetic cluster sequence, e.g., at least 5%, 10%, 15%, 20%, 25%, 30%, 35%, 40%, 50% or more of the full length naturally occurring genetic cluster sequence). In some embodiments, the nucleotide sequence is at least about 90%, 91%, 92%, 93%, 94%, 95%, 96%, 97%, 98%, 99% or 100% homologous or identical relative to a naturally occurring genetic cluster sequence. In some embodiments, the nucleotide sequence is at least about 85%, e.g., is at least about 90%, 91%, 92%, 93%, 94%, 95%, 96%, 97%, 98%, 99% or 100% homologous or identical to a genetic cluster sequence, in a fragment thereof or a region that is much more conserved, such as an essential, but has lower sequence identity outside that region.

Calculations of homology or sequence identity between sequences (the terms are used interchangeably herein) may be performed as follows. To determine the percent identity of two nucleic acid sequences, the sequences may be aligned for optimal comparison purposes (e.g., gaps can be introduced in one or both of a first and a second amino acid or nucleic acid sequence for optimal alignment and non-homologous sequences can be disregarded for comparison purposes). The length of a reference sequence aligned for comparison purposes is at least 80% of the length of the reference sequence, and in some embodiments is at least 90% or 100%. The nucleotides at corresponding amino acid positions or nucleotide positions may be then compared. When a position in the first sequence is occupied by the same nucleotide as the corresponding position in the second sequence, then the molecules are identical at that position (as used herein nucleic acid “identity” is equivalent to nucleic acid “homology”). The percent identity between the two sequences is a function of the number of identical positions shared by the sequences, taking into account the number of gaps, and the length of each gap, which need to be introduced for optimal alignment of the two sequences.

In some embodiments the gene clusters may be native gene clusters. In some embodiments, the gene clusters may be refactored gene clusters. A refactored gene cluster is a gene cluster which is synthetic and has one or more distinctions from a naturally occurring gene cluster. In other words an engineered gene cluster has some alteration relative to a naturally occurring gene cluster, i.e. its genes are reordered, deleted or altered in some way relative to a corresponding natural gene cluster. A target, naturally occurring, or wild type genetic cluster is one which serves as the original model for the refactoring.

As used herein, the equivalent terms “expression” or “gene expression” are intended to refer to the transcription of a DNA molecule into RNA, and/or the translation of such RNA into a polypeptide.

As used herein, a “gene cluster” refers to a set of two or more genes that encode gene products. As used herein, a “nif gene cluster” refers to a set of two or more genes that encode nitrogen fixation genes.

“Exogenous” with respect to genes indicates that the nucleic acid or gene is not in its natural (native) environment. For example, an exogenous gene can refer to a gene that is from a different species. In contrast, “endogenous” with respect to genes indicates that the gene is in its native environment. As used herein, the terms “endogenous” and “native” are used interchangeably.

As used herein, the term “delete” or “deleted” refers to the removal of a gene (e.g. endogenous gene) from a sequence or cluster. As used herein, the term “alter” or “altered” refers to the modification of one or more nucleotides in a gene or the deletion of one or more base pairs in a gene. This alteration may render the gene dysfunctional. Method of deletion and alteration, in the context of genes, are known in the art.

The term “gene” may refer to chromosomal DNA, plasmid DNA, cDNA, synthetic DNA, or other DNA that encodes a peptide, polypeptide, protein, or RNA molecule, and regions flanking the coding sequence involved in the regulation of expression. Some genes can be transcribed into mRNA and translated into polypeptides (structural genes); other genes can be transcribed into RNA (e.g., rRNA, tRNA); and other types of genes function as regulators of expression (regulator genes).

“Expression” of a gene refers to the transcription of a gene to produce the corresponding mRNA and/or translation of this mRNA to produce the corresponding gene product, i.e., a peptide, polypeptide, or protein. Gene expression may be controlled or modulated by regulatory elements including 5′ regulatory elements such as promoters.

“Genetic component” refers to any nucleic acid sequence or genetic element that may also be a component or part of an expression vector. Examples of genetic components include, but are not limited to, promoter regions, 5′ untranslated leaders, introns, genes, 3′ untranslated regions, and other regulatory sequences or sequences that affect transcription or translation of one or more nucleic acid sequences.

In certain exemplary embodiments, vectors such as, for example, expression vectors, containing a nucleic acid encoding one or more mRNAs or reporter polypeptides and/or proteins described herein are provided. As used herein, the term “vector” refers to a nucleic acid molecule capable of transporting another nucleic acid to which it has been linked. One type of vector is a “plasmid,” which refers to a circular double stranded DNA loop into which additional DNA segments can be ligated. Such vectors are referred to herein as “expression vectors.” In general, expression vectors of utility in recombinant DNA techniques are often in the form of plasmids. In the present specification, “plasmid” and “vector” can be used interchangeably. However, the disclosed methods and compositions are intended to include such other forms of expression vectors, such as viral vectors (e.g., replication defective retroviruses, adenoviruses and adeno-associated viruses), which serve equivalent functions.

In certain exemplary embodiments, the recombinant expression vectors comprise a nucleic acid sequence (e.g., a nucleic acid sequence encoding one or more mRNAs or reporter polypeptides and/or proteins described herein) in a form suitable for expression of the nucleic acid sequence in one or more of the methods described herein, which means that the recombinant expression vectors include one or more regulatory sequences which is operatively linked to the nucleic acid sequence to be expressed. Within a recombinant expression vector, “operably linked” is intended to mean that the nucleotide sequence encoding one or more mRNAs or reporter polypeptides and/or proteins described herein is linked to the regulatory sequence(s) in a manner which allows for expression of the nucleotide sequence (e.g., in an in vitro ribosomal assembly, transcription and/or translation system). The term “regulatory sequence” is intended to include promoters, enhancers and other expression control elements (e.g., polyadenylation signals). Such regulatory sequences are described, for example, in Goeddel; Gene Expression Technology: Methods in Enzymology 185, Academic Press, San Diego, Calif. (1990).

The genetic elements described herein can each be under the control of a regulatory element, such as an inducible or repressible promoter or enhancer element. In some embodiments, one or more genes are under the control of the same or different regulatory elements. In some embodiments, all the genes are under the control of the same or different regulatory elements.

The regulatory elements may also be activation elements or inhibitory elements. An activation element is a nucleic acid sequence that when presented in context with a nucleic acid to be expressed will cause expression of the nucleic acid in the presence of an activation signal. An inhibitory signal is a nucleic acid sequence that when presented in context with a nucleic acid to be expressed will cause expression of the nucleic acid unless an inhibitory signal is present. Each of the activation and inhibitory elements may be a promoter, such as a bacteriophage T7 promoter, sigma 70 promoter, sigma 54 promoter, lac promoter, etc. As used herein, the term “promoter” is intended to refer to those regulatory sequences which are sufficient to enable the transcription of an operably linked DNA molecule. Promoters may be constitutive or inducible. As used herein, the term “constitutive promoter” refers to a promoter that is always on (i.e. causing transcription at a constant level). Examples of constitutive promoters include, without limitation, sigma 70 promoter, bla promoter, lacI. promoter, etc. Non-limiting examples of inducible promoters are shown in the following table:

| | | Essential Regulatory |
|---|---|---|
| Name | Chemical Inducer and/or Repressor | Gene(s) |
| ParaBAD | L-arabinose (ON) & glucose (OFF) | araC |
| (“PBAD”) | | |
| PrhaBAD | L-Rhamnose (ON) & glusoe (OFF) | rhaR & rhaS |
| Plac | lactose or Iptg (on) & glucose (OFF) | lacI |
| Ptac | lactose or IPTG (ON) | lacI |
| Plux | acyl-homoserine lactone (ON) | luxR |
| Ptet | tetracycline or aTc (ON) | tetR |
| Psal | salycilate (ON) | nahR |
| Ptrp | tryptophan (OFF) | (NONE) |
| Ppho | phosphate (OFF) | phoB & phoR |

Inducible promoters allow regulation of gene expression and can be regulated by exogenously supplied compounds, environmental factors such as temperature, or the presence of a specific physiological state, e.g., acute phase, a particular differentiation state of the cell, or in replicating cells only. Inducible promoters and inducible systems are available from a variety of commercial sources, including, without limitation, Invitrogen, Clontech and Ariad. Many other systems have been described and can be readily selected by one of skill in the art. Examples of inducible promoters regulated by exogenously supplied promoters include the zinc-inducible sheep metallothionine (MT) promoter, the dexamethasone (Dex)-inducible mouse mammary tumor virus (MMTV) promoter, the T7 polymerase promoter system [WO 98/10088]; the ecdysone insect promoter [No et al, Proc. Natl. Acad. Sci. USA, 93:3346-3351 (1996)], the tetracycline-repressible system [Gossen et al, Proc. Natl. Acad. Sci. USA, 89:5547-5551 (1992)], the tetracycline-inducible system [Gossen et al, Science, 268:1766-1769 (1995), see also Harvey et al, Curr. Opin. Chem. Biol., 2:512-518 (1998)], the RU486-inducible system [Wang et al, Nat. Biotech., 15:239-243 (1997) and Wang et al, Gene Ther., 4:432-441 (1997)] and the rapamycin-inducible system [Magari et al, J. Clin. Invest., 100:2865-2872 (1997)]. Still other types of inducible promoters which may be useful in this context are those which are regulated by a specific physiological state, e.g., temperature, acute phase, a particular differentiation state of the cell, or in replicating cells only.

The components and methods disclosed herein may include and/or utilize chemical signals. As used herein, the term “chemical signals” refers to chemical compounds. Any substance consisting of two or more different types of atoms (chemical elements) in a fixed stoichiometric proportion can be termed a chemical compound. Chemical signals can be synthetic or natural chemical compounds. In some embodiments of the present invention, a bacterium of the present disclosure or a sensor of the present disclosure is under the control of a chemical signal. In some embodiments, the signal is a native biological signal (e.g. root exudate, biological control agent, etc.). In some embodiments, the chemical signal is a quorum sensing signal from the bacterium. Non-limiting examples of chemical signals include biocontrol agents, phytohormones, vanillate, IPTG, aTc, cuminic acid, DAPG, and salicylic acid, 3,4-dihydroxybenzoic acid, 3OC6HSL and 3OC14HSL.

As used herein, the term “phytohormone” refers plant hormones and they are any of various hormones produced by plants that influence process such as germination, growth, and metabolism in the plant.

The disclosed components and methods may include or utilize terminators. As used herein, the term “terminator” (as referred to as a transcription terminator) is a section of nucleic acid sequence that marks the end of a gene or operon in genomic DNA during transcription. They stop transcription of a polymerase. Terminators can be classified into several groups. At the first group of termination signals the core enzyme can terminate in vitro at certain sites in the absence of any other factors (as tested in vitro). These sites of termination are called intrinsic terminators or also class I terminators. Intrinsic terminators usually share one common structural feature, the so called hairpin or stem-loop structure. On the one hand the hairpin comprises a stem structure, encoded by a dG-dC rich sequence of dyad symmetrical structure. On the other hand the terminator also exhibits a dA-dT rich region at the 3′-end directly following the stem structure. The uridine rich region at the 3′ end is thought to facilitate transcript release when RNA polymerase pauses at hairpin structures. Two or more terminators can be operatively linked if they are positioned to each other to provide concerted termination of a preceding coding sequence. Particularly preferred, the terminator sequences are downstream of coding sequences, i.e. on the 3′ position of the coding sequence. The terminator can e.g. be at least 1, at least 10, at least 30, at least 50, at least 100, at least 150, at least 200, at least 250, at least 300, at least 400, at least 500 nucleotides downstream of the coding sequence or directly adjacent. Examples of terminators include, but are not limited to, T7 terminator, rmBT1, L3S2P21, tonB, rrnA, rrnB, rnD, RNAI, crp, his, ilv lambda, M13, rpoC, and trp (see for example U.S. Pat. No. 9,745,588, incorporated herein by reference).

In some instances, the nucleic acids disclosed herein may include non-naturally occurring nucleotides and/or substitutions, i.e. Sugar or base substitutions or modifications. One or more substituted sugar moieties include, e.g., one of the following at the 2′ position: OH, SH, SCH₃, F, OCN, OCH₃OCH₃, OCH₃O(CH₂)n CH₃, O(CH₂)n NH₂ or O(CH₂)ₙCH₃ where n is from 1 to about 10; C₁ to C₁₀ lower alkyl, alkoxyalkoxy, substituted lower alkyl, alkaryl or aralkyl; Cl; Br; CN; CF₃; OCF₃; O-, S-, or N-alkyl; O-, S-, or N-alkenyl; SOCH₃; SO₂; CH₃; ONO₂; NO₂; N₃; NH₂; heterocycloalkyl; heterocycloalkaryl; aminoalkylamino; polyalkylamino; substituted silyl; an RNA cleaving group; a reporter group; an intercalator; a group for improving the pharmacokinetic properties of a nucleic acid; or a group for improving the pharmacodynamic properties of a nucleic acid and other substituents having similar properties. Similar modifications may also be made at other positions on the nucleic acid, particularly the 3′ position of the sugar on the 3′ terminal nucleotide and the 5′ position of 5′ terminal nucleotide. Nucleic acids may also have sugar mimetics such as cyclobutyls in place of the pentofuranosyl group.

Nucleic acids can also include, additionally or alternatively, nucleobase (often referred to in the art simply as “base”) modifications or substitutions. As used herein, “unmodified” or “natural” nucleobases include adenine (A), guanine (G), thymine (T), cytosine (C) and uracil (U). Modified nucleobases include nucleobases found only infrequently or transiently in natural nucleic acids, e.g., hypoxanthine, 6-methyladenine, 5-Me pyrimidines, particularly 5-methylcytosine (also referred to as 5-methyl-2′ deoxycytosine and often referred to in the art as 5-Me-C), 5-hydroxymethylcytosine (HMC), glycosyl HMC and gentobiosyl HMC, isocytosine, pseudoisocytosine, as well as synthetic nucleobases, e.g., 2-aminoadenine, 2-(methylamino)adenine, 2-(imidazolylalkyl)adenine, 2-(aminoalklyamino)adenine or other heterosubstituted alkyladenines, 2-thiouracil, 2-thiothymine, 5-bromouracil, 5-hydroxymethyluracil, 5-propynyluracil, 8-azaguanine, 7-deazaguanine, N6 (6-aminohexyl)adenine, 6-aminopurine, 2-aminopurine, 2-chloro-6-aminopurine and 2,6-diaminopurine or other diaminopurines. See, e.g., Komberg, “DNA Replication,” W. H. Freeman & Co., San Francisco, 1980, pp 75-′7′ 7; and Gebeyehu, G., et al. Nucl. Acids Res., 15:4513 (1987)). A “universal” base known in the art, e.g., inosine, can also be included.

Illustrative and non-limiting examples of modified nucleotides include, but are not limited to diaminopurine, S²T, 5-fluorouracil, 5-bromouracil, 5-chlorouracil, 5-iodouracil, hypoxanthine, xantine, 4-acetylcytosine, 5-(carboxyhydroxylmethyl)uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-D46-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, 2,6-diaminopurine and the like. Nucleic acid molecules may also be modified at the base moiety (e.g., at one or more atoms that typically are available to form a hydrogen bond with a complementary nucleotide and/or at one or more atoms that are not typically capable of forming a hydrogen bond with a complementary nucleotide), sugar moiety or phosphate backbone.

Peptides, Polypeptides, Proteins, and Synthesis Methods

As used herein, the terms “peptide,” “polypeptide,” and “protein,” refer to molecules comprising a chain a polymer of amino acid residues joined by amide linkages. The term “amino acid residue,” includes but is not limited to amino acid residues contained in the group consisting of alanine (Ala or A), cysteine (Cys or C), aspartic acid (Asp or D), glutamic acid (Glu or E), phenylalanine (Phe or F), glycine (Gly or G), histidine (His or H), isoleucine (Ile or I), lysine (Lys or K), leucine (Leu or L), methionine (Met or M), asparagine (Asn or N), proline (Pro or P), glutamine (Gln or Q), arginine (Arg or R), serine (Ser or S), threonine (Thr or T), valine (Val or V), tryptophan (Trp or W), and tyrosine (Tyr or Y) residues. The term “amino acid residue” also may include nonstandard, noncanonical, or unnatural amino acids, which optionally may include amino acids other than any of the following amino acids: alanine, cysteine, aspartic acid, glutamic acid, phenylalanine, glycine, histidine, isoleucine, lysine, leucine, methionine, asparagine, proline, glutamine, arginine, serine, threonine, valine, tryptophan, and tyrosine residues. The term “amino acid residue” may include alpha-, beta-, gamma-, and delta-amino acids.

In some embodiments, the term “amino acid residue” may include nonstandard, noncanonical, or unnatural amino acid residues contained in the group consisting of homocysteine, 2-Aminoadipic acid, N-Ethylasparagine, 3-Aminoadipic acid, Hydroxylysine, β-alanine, β-Amino-propionic acid, allo-Hydroxylysine acid, 2-Aminobutyric acid, 3-Hydroxyproline, 4-Aminobutyric acid, 4-Hydroxyproline, piperidinic acid, 6-Aminocaproic acid, Isodesmosine, 2-Aminoheptanoic acid, allo-Isoleucine, 2-Aminoisobutyric acid, N-Methylglycine, sarcosine, 3-Aminoisobutyric acid, N-Methylisoleucine, 2-Aminopimelic acid, 6-N-Methyllysine, 2,4-Diaminobutyric acid, N-Methylvaline, Desmosine, Norvaline, 2,2′-Diaminopimelic acid, Norleucine, 2,3-Diaminopropionic acid, Ornithine, and N-Ethylglycine. The term “amino acid residue” may include L isomers or D isomers of any of the aforementioned amino acids.

Other examples of nonstandard, noncanonical, or unnatural amino acids include, but are not limited, to a p-acetyl-L-phenylalanine, a p-iodo-L-phenylalanine, an O-methyl-L-tyrosine, a p-propargyloxyphenylalanine, a p-propargyl-phenylalanine, an L-3-(2-naphthyl)alanine, a 3-methyl-phenylalanine, an O-4-allyl-L-tyrosine, a 4-propyl-L-tyrosine, a tri-O-acetyl-GlcNAcpβ-serine, an L-Dopa, a fluorinated phenylalanine, an isopropyl-L-phenylalanine, a p-azido-L-phenylalanine, a p-acyl-L-phenylalanine, a p-benzoyl-L-phenylalanine, an L-phosphoserine, a phosphonoserine, a phosphonotyrosine, a p-bromophenylalanine, a p-amino-L-phenylalanine, an isopropyl-L-phenylalanine, an unnatural analogue of a tyrosine amino acid; an unnatural analogue of a glutamine amino acid; an unnatural analogue of a phenylalanine amino acid; an unnatural analogue of a serine amino acid; an unnatural analogue of a threonine amino acid; an unnatural analogue of a methionine amino acid; an unnatural analogue of a leucine amino acid; an unnatural analogue of a isoleucine amino acid; an alkyl, aryl, acyl, azido, cyano, halo, hydrazine, hydrazide, hydroxyl, alkenyl, alkynl, ether, thiol, sulfonyl, seleno, ester, thioacid, borate, boronate, 27ufa27hor, phosphono, phosphine, heterocyclic, enone, imine, aldehyde, hydroxylamine, keto, or amino substituted amino acid, or a combination thereof; an amino acid with a photoactivatable cross-linker; a spin-labeled amino acid; a fluorescent amino acid; a metal binding amino acid; a metal-containing amino acid; a radioactive amino acid; a photocaged and/or photoisomerizable amino acid; a biotin or biotin-analogue containing amino acid; a keto containing amino acid; an amino acid comprising polyethylene glycol or polyether; a heavy atom substituted amino acid; a chemically cleavable or photocleavable amino acid; an amino acid with an elongated side chain; an amino acid containing a toxic group; a sugar substituted amino acid; a carbon-linked sugar-containing amino acid; a redox-active amino acid; an α-hydroxy containing acid; an amino thio acid; an α,α disubstituted amino acid; a β-amino acid; a γ-amino acid, a cyclic amino acid other than proline or histidine, and an aromatic amino acid other than phenylalanine, tyrosine or tryptophan.

As used herein, a “peptide” is defined as a short polymer of amino acids, of a length typically of 20 or less amino acids, and more typically of a length of 12 or less amino acids (Garrett & Grisham, Biochemistry, 2^{nd} edition, 1999, Brooks/Cole, 110). In some embodiments, a peptide as contemplated herein may include no more than about 2, 3, 4, 5, 6, 7, 8, 9, 10, 11, 12, 13, 14, 15, 16, 17, 18, 19, or 20 amino acids. A polypeptide, also referred to as a protein, is typically of length ≥100 amino acids (Garrett & Grisham, Biochemistry, 2^{nd} edition, 1999, Brooks/Cole, 110). A polypeptide, as contemplated herein, may comprise, but is not limited to, 100, 101, 102, 103, 104, 105, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000, about 2250, about 2500 or more amino acid residues.

A peptide as contemplated herein may be further modified to include non-amino acid moieties. Modifications may include but are not limited to acylation (e.g., O-acylation (esters), N-acylation (amides), S-acylation (thioesters)), acetylation (e.g., the addition of an acetyl group, either at the N-terminus of the protein or at lysine residues), formylation lipoylation (e.g., attachment of a lipoate, a C8 functional group), myristoylation (e.g., attachment of myristate, a C14 saturated acid), palmitoylation (e.g., attachment of palmitate, a C16 saturated acid), alkylation (e.g., the addition of an alkyl group, such as an methyl at a lysine or arginine residue), isoprenylation or prenylation (e.g., the addition of an isoprenoid group such as famesol or geranylgeraniol), amidation at C-terminus, glycosylation (e.g., the addition of a glycosyl group to either asparagine, hydroxylysine, serine, or threonine, resulting in a glycoprotein). Distinct from glycation, which is regarded as a nonenzymatic attachment of sugars, polysialylation (e.g., the addition of polysialic acid), glypiation (e.g., glycosylphosphatidylinositol (GPI) anchor formation, hydroxylation, iodination (e.g., of thyroid hormones), and phosphorylation (e.g., the addition of a phosphate group, usually to serine, tyrosine, threonine or histidine).

As used herein, “translation template” refers to an RNA product of transcription from an expression template that can be used by ribosomes to synthesize polypeptides or proteins.

The term “reaction mixture,” as used herein, refers to a solution containing reagents necessary to carry out a given reaction. A reaction mixture is referred to as complete if it contains all reagents necessary to perform the reaction. Components for a reaction mixture may be stored separately in separate container, each containing one or more of the total components. Components may be packaged separately for commercialization and useful commercial kits may contain one or more of the reaction components for a reaction mixture.

The steps of the methods described herein can be performed in any suitable order unless otherwise indicated herein or otherwise clearly contradicted by context. The steps may be repeated or reiterated any number of times to achieve a desired goal unless otherwise indicated herein or otherwise clearly contradicted by context.

Preferred aspects of this invention are described herein, including the best mode known to the inventors for carrying out the invention. Variations of those preferred aspects may become apparent to those of ordinary skill in the art upon reading the foregoing description. The inventors expect a person having ordinary skill in the art to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Cell-Free Protein Synthesis

The disclosed components, compositions, systems, kits, and methods may be utilized for performing cell-free protein synthesis. Cell-free protein synthesis (CFPS) exploits an ensemble of catalytic proteins prepared from the crude lysate of cells. The cell extract (whose composition is sensitive to growth media, lysis method, and processing conditions) is an important component of extract-based CFPS reactions. A variety of methods exist for preparing an extract competent for cell-free protein synthesis, including those described herein.

Cell-free protein synthesis (CFPS) and methods for making cell extracts for use in CFPS are known in the art. (See, e.g., Carlson et al., “Cell-free protein synthesis: Applications come of age,” Biotech. Adv. Vol. 30, Issue 5, September-October 2012, Pages 1185-1194; Hodgman et al., “Cell-free synthetic biology: Thinking outside the cell,” Metabol. Eng. Vol. 14, Issue 3, May 2012, Pages 261-269; and Harris et al., “Cell-free biology: exploiting the interface between synthetic biology and synthetic chemistry,” Curr. Op. Biotech. Vol. 23, Issue 5, October 2012, Pages 672-678; see also U.S. Pat. Nos. 7,312,049; 7,008,651; and 6,994,986; see also U.S. Published Application Nos. 20170306320; 20160362708; 20160060301; 20120088269; 20090042244; 2008024821; 20080138857; 20070154983; 20070141661; 20050186655; 20050148046120050064592; 20050032086; 20040209321; and 20040038332; the contents of which are incorporated herein by reference in their entireties).

The disclosed compositions may include platforms for preparing a sequence defined biopolymer of protein in vitro. The platforms for preparing a sequence defined polymer or protein in vitro comprises an extract from an organism, and in particular a species of plant, such as an extract from chloroplasts of a plant, such as Nicotiana tabacum.

Because CFPS exploits an ensemble of catalytic proteins prepared from the crude lysate of cells, the cell extract (whose composition is sensitive to growth media, lysis method, and processing conditions) is an important component of extract-based CFPS reactions. A variety of methods exist for preparing an extract competent for cell-free protein synthesis, including those disclosed in U.S. Published Application No. 20140295492, published on Oct. 2, 2014, which is incorporated by reference.

The platform may comprise an expression template, a translation template, or both an expression template and a translation template. The expression template serves as a substrate for transcribing at least one RNA that can be translated into a sequence defined biopolymer (e.g., a polypeptide or protein). The translation template is an RNA product that can be used by ribosomes to synthesize the sequence defined biopolymer. In certain embodiments the platform comprises both the expression template and the translation template. In certain specific embodiments, the platform may be a coupled transcription/translation (“Tx/Tl”) system where synthesis of translation template and a sequence defined biopolymer from the same cellular extract.

The platform may comprise one or more polymerases capable of generating a translation template from an expression template. The polymerase may be supplied exogenously or may be supplied from the organism used to prepare the extract. In certain specific embodiments, the polymerase is expressed from a plasmid present in the organism used to prepare the extract and/or an integration site in the genome of the organism used to prepare the extract.

The platform may comprise an orthogonal translation system. An orthogonal translation system may comprise one or more orthogonal components that are designed to operate parallel to and/or independent of the organism's orthogonal translation machinery. In certain embodiments, the orthogonal translation system and/or orthogonal components are configured to incorporation of unnatural amino acids. An orthogonal component may be an orthogonal protein or an orthogonal RNA. In certain embodiments, an orthogonal protein may be an orthogonal synthetase. In certain embodiments, the orthogonal RNA may be an orthogonal tRNA or an orthogonal rRNA. An example of an orthogonal rRNA component has been described in U.S. Published Application Nos. 20170073381 and 20160060301, the contents of which are incorporated by reference in their entireties. In certain embodiments, one or more orthogonal components may be prepared in vivo or in vitro by the expression of an oligonucleotide template. The one or more orthogonal components may be expressed from a plasmid present in the genomically recoded organism, expressed from an integration site in the genome of the genetically recoded organism, co-expressed from both a plasmid present in the genomically recoded organism and an integration site in the genome of the genetically recoded organism, express in the in vitro transcription and translation reaction, or added exogenously as a factor (e.g., a orthogonal tRNA or an orthogonal synthetase added to the platform or a reaction mixture.

Platforms Comprising Extracts from Plant Chloroplasts

The disclosed compositions (or systems) may include platforms for preparing a sequence defined biopolymer or protein in vitro, where the platform comprising an extract prepared from plant chloroplast, in particular, plant chloroplasts from Nicotiana tabacum and maize.

The platforms disclosed herein may include additional components, for example, one or more components for performing CFPS. Components may include, but are not limited to amino acids which optionally may include noncanonical amino acids, NTPs, salts (e.g., sodium salts, potassium salts, and/or magnesium salts), cofactors (e.g., nicotinamide adenine dinucleotide (NAD) and/or coenzyme-A (CoA)), an energy source and optionally an energy source comprising a phosphate group (e.g., phosphoenol pyruvate (PEP), ATP, or creatine phosphate), a translation template (e.g., a non-native mRNA that is translated in the platform) and/or a transcription template (e.g., a template DNA for synthesizing a non-native mRNA that is translated in the platform), and any combination thereof.

In some embodiments, the platform may comprise an energy source and optionally an energy source comprising a phosphate group (e.g., phosphoenol pyruvate (PEP), ATP, or creatine phosphate), where the energy source is present in the platform at a concentration of greater than about 30 mM, 40 mM, 50 mM, 60 mM, 70 mM, 80 mM, or 90 mM (preferably greater than about 67 mM), but less than about 100 mM, or within a concentration range bounded by of these values.

In some embodiments, the platform further comprises a source of potassium (K⁺)(such as a potassium salt such as potassium glutamate), where the platform comprises potassium at a concentration greater than about 50 mM, 100 mM, 150 mM, 200 mM, 250 mM, 300 mM, 350 mM, 400 mM, or 450 mM (preferably about 300 mM), but less than about 500 mM, or within a concentration range bounded by of these values,

The disclosed platforms and cell extracts may be utilized in methods for preparing a sequence defined biopolymer or protein in vitro. The disclosed methods typically include translating in vitro a translation template (e.g., mRNA) encoding the sequence defined biopolymer or protein in the platform of any of the foregoing claims. Optionally, the disclosed methods may include transcribing a transcription template (e.g., DNA) in the platform to provide the translation template.

The disclosed methods may be performed under conditions that are suitable for extracts prepared from plant chloroplasts. In some embodiments, the disclosed methods are performed at a temperature between about 20-40° C., and preferably at a temperature of about 30° C.

The disclosed methods may be performed to synthesize any sequence defined biopolymer or protein. In some embodiments, the sequence defined polymer or protein is a therapeutic protein and/or the method may utilized to identify therapeutic proteins or biomaterials by translating a library of transcription templates. In some embodiments, the disclosed methods may be performed to optimize in vitro translation conditions for a cellular extract prepared from a species of plant.

Kits also are contemplated herein. In some embodiments, the contemplated kits comprise as components: (a) a cellular extract prepared from plant chloroplasts; and (b) a reaction mixture for transcribing and/or translating an mRNA. Suitable components for the reaction mixture of the disclosed kits may include, but are not limited to, amino acids which optionally may include noncanonical amino acids, NTPs, salts (e.g., sodium salts, potassium salts, and/or magnesium salts), cofactors (e.g., nicotinamide adenine dinucleotide (NAD) and/or coenzyme-A (CoA)), an energy source and optionally an energy source comprising a phosphate group (e.g., ATP or creatine phosphate).

Maize Plastid Cell-Free System and Uses Thereof for Prototyping Gene Expression in a

A method for prototyping expression of single genes, multi-genic constructs and characterization of genetic parts in a plastid cell-free system purified from maize is provided. The maize plastid cell-free system is a high throughput system that may enable 10,000s or more genetic constructs to be prototyped weekly, thus, speeding up the design and test cycle of maize engineering and preventing mistakes like using incorrect codon-optimization or ribosome binding sites. Those genetic designs producing the desired expression pattern can be selected for maize plastid transformation.

Plants due to their slow regeneration and long generation times are considered to be difficult to engineer. Genetic prototyping in cell-free systems has been demonstrated herein, enabling the testing of many constructs in a short period of time without the constraints of in vivo engineering. The viability of the cell-free technology is demonstrated in a purified plant (maize). This technology may revolutionize maize biotechnology by enabling plant engineers to prototype several design variants very quickly in a high throughput manner. Constructs producing the desired expression pattern in vitro can be selected for maize plastid transformation which will reduce the amount of work related to plant regeneration, increase the number of tested designs resulting in successful engineering of complex functions in maize.

These techniques enable the pre-screening of DNA designs with a maize plastid cell-free system to allow for a dramatic reduction in iteration time for research and development. 10,000 or more designs can be tested in a week which would be impossible in vivo. High-throughput interrogation and characterization of organismal genetic parts (promoters, terminators, ribosome binding sites, etc.) are also enabled. Other important uses include in vitro screening of enzyme pathway variants for plant derived metabolites, synthetic biology applications, fundamental studies of the plastid translation apparatus and harnessing translation machinery for novel functions, agriculture, and crop science.

Maize plastid transformation is a laborious process that involves bombardment of plant tissue with gold particles loaded with DNA and regeneration of the plant tissue into a new plant. This method is not sufficiently high-throughput for prototyping genetic parts or multi-genic constructs. Maize plastid transformation takes approximately 3-6 months to produce transformants, meaning that design iterations are heavily time-constrained (2/year).

Plant plastids (chloroplasts, amyloplasts, elaioplasts, chromoplasts, etc.) are the major biosynthetic centers that in addition to photosynthesis are responsible for production of industrially important compounds such as amino acids, complex carbohydrates, fatty acids, and pigments. Plastids are derived from a common precursor known as a proplastid and thus the plastids present in a given plant species all have the same genetic content. Plant cells contain 500-10,000 copies of a small 120-160 kilobase circular genome, each molecule of which has a large (approximately 25 kb) inverted repeat. Thus, it is possible to engineer plant cells to contain up to 20,000 copies of a particular gene of interest, which potentially can result in very high levels of foreign gene expression.

The components, compositions, systems, kits, and method disclosed herein may be utilized to prepare genetically modified maize having genetically modified plant plastids accordingly. The genetically modified maize may exhibit one or more desirable phenotypes in which expression of one or more genes are modulated in the genetically modified maize.

### Illustrative Embodiments

The following Embodiments are illustrative and should not be interpreted to limit the scope of the claimed subject matter.

1. A component, composition, system, kit, or method for use in performing in vitro transcription of mRNA and/or in vitro translation of polypeptides. The component comprising or the composition, system, kit or method comprising or utilizing as a component: (a) a plastid extract prepared from plants (e.g., a plastid extract prepared from isolated intact chloroplasts), optionally plants that are grown for about 1-8 weeks, for about 2-8 weeks, or for about 4-8 weeks, optionally wherein the plants are grown in about 6 hours dark and in about 18 hours light during the 1-8 weeks, or the 2-8 weeks, or the 4-8 weeks, and optionally incubating the plants in total dark for 24 hours, preferably 10-12 hours before preparing the plastid extract. The component optionally comprising or the composition, system, kit or method optionally comprising or utilizing as a component one or more of the following components: (b) a reaction buffer; (c) an RNA polymerase; and (d) the transcription template, wherein the RNA polymerase is capable of transcribing the transcription template to form a translation template and the plastid extract can sustain protein synthesis through a combined transcription/translation reaction. Optionally, the plastid extract is prepared by lysing cells of the plants and isolating intact plastids from the lysed cells, and subsequently lysing the intact plastids and separating the plastid extract from the lysed plastids (e.g., which may contain plastid membranes and other water insoluble components). The plastid extract may be obtained by subjecting the lysed plastids to centrifugation and collecting the supernatant as the plastid extract.

2. The component, composition, system, kit, or method of claim 1, wherein the plastid extract is prepared from isolated intact chloroplasts.

3. The component, composition, system, kit, or method of claim 1, wherein the plastid extract is prepared from a species selected from Nicotiana spp. (e.g., Nicotiana tobaccum).

4. The component, composition, system, kit, or method of embodiment 1, wherein the plastid extract is prepared from a species selected from Maize spp (e.g., Zea mays L.).

5. The component, composition, system, kit, or method of embodiment 1, wherein the plastid extract is prepared from a species selected from Glycine spp. (e.g. soybean plant), Oryza spp. (e.g., rice plant), and Triticum spp. (e.g., wheat plant).

6. The component, composition, system, kit, or method of embodiment 1, wherein the plastid extract is prepared from a species selected from Solanum spp. (e.g., Solanum lycopersicum (tomato plant) or Solanum tubersosum (potato plant)).

7. The component, composition, system, kit, or method of any of embodiments 1-6, wherein the plastid extract is prepared from a plant that is engineered to be deficient in a negative effector for cell-free protein synthesis (CFPS), or from a plant that is engineered to be deficient in a negative effector for combined transcription and translation.

8. The component, composition, system, kit, or method of any of the foregoing embodiments, wherein the plastid extract is prepared from a plant that has been engineered to express T7 RNA polymerase in the chloroplast or in the nucleus and T7 RNA polymerase is targeted to the chloroplast.

9. The component, composition, system, kit, or method of any of the foregoing embodiments, wherein the component (e.g., the plastid extract) or the composition, system, or kit is preserved, such as for example through freeze-drying.

10. A method for in vitro transcription of mRNA and/or translation of mRNA to prepare a polypeptide, the method comprising reacting a reaction mixture comprising: (a) an extract from plastids of a plant, wherein the plant optionally is grown for about 1-8 weeks, or for about 2-8 weeks, or for about 4-8 weeks and preferably for about 6 weeks; (b) a template for transcription of the mRNA (e.g., a DNA encoding the mRNA which may be linear or circular such as a plasmid) and/or a template for translation to prepare the polypeptide (e.g., an mRNA); (c) monomers for synthesis of the mRNA (e.g., NTPs such as one or more of ATP, CTP, GTP, and/or UTP) and/or the polypeptide (e.g., one or more of any of the twenty (20) canonical amino acids or non-canonical amino acids); (d) co-factors, enzymes and/or other reagents necessary for the transcription and/or translation; and (e) magnesium at a concentration of from about 3 mM to about 20 mM (or from about 4 mM to about 12 mM, or from about 6 mM to about 10 mM), optionally wherein the method is performed using an automated system and/or mechanized system which optionally comprises a liquid-handling robot, for example, to assemble the reaction mixture.

11. The method of embodiment 10, wherein the concentration of NTPs (as a group or individually) is between about 0.5 to 4 mM and/or wherein the concentration of amino acids (as a group or individually) is between about 0.5 to 4 mM, and preferably about 2 mM.

12. The method of embodiment 10 or 11, wherein the reaction mixture further comprises potassium at a concentration of about 0-200 mM, and preferably about 100 mM.

13. The method of any of embodiments 10-12, wherein the reaction mixture further comprises salts at a total concentration of between about 50-400 mM.

14. The method of any of embodiments 10-13, wherein the reaction mixture further comprises an energy regeneration system that comprises creatine kinase and creatine phosphate.

15. The method of any of embodiments 10-14, wherein the reaction mixture further comprises at least one macromolecular crowding agent (e.g., polyethylene glycol or Ficol).

16. The method of any of embodiments 10-15, wherein the plastid extract is prepared by a method that includes a step of adding glycerol to the plastids prior to lysing the plastids (e.g., lysis by freezing), preferably a step of adding glycerol at a concentration of about 5-15% or about 10% to the plastids prior to lysing the plastids.

17. The method of any of embodiments 10-146, wherein the template for the mRNA includes modifications in the 3′UTR that facilitate efficient transcription and/or translation.

18. The method of any of embodiments 10-17, wherein the template for the mRNA includes modifications in the 5′UTR that facilitate efficient transcription and/or translation.

19. The method of any of embodiments 10-18, wherein the reaction mixture further comprises a DNA-dependent RNA polymerase.

20. The method of embodiment 19, wherein the DNA-dependent RNA polymerase is a bacteriophage DNA-dependent RNA, such as T7 RNA polymerase.

21. The method of any of embodiments 10-20, wherein the method is performed as a batch reaction.

22. The method of any of embodiments 10-20, wherein the method is performed as a semi-continuous reaction.

23. The method of any of embodiments 10-20, wherein the method is performed as a semi-continuous reaction.

24. The method of any of embodiments 10-23, wherein the method is performed at a temperature between about 20-40° C.

25. The method of any of embodiments 10-24, wherein the reaction mixture or any component of the reaction mixture (e.g., the extract from plastids) is preserved, such as for example through freeze-drying and is rehydrated for use in the method.

26. The method of any of embodiments 10-25, wherein an RNAse inhibitor is added to the reaction mixture to enhance

27. A method comprising: (a) creating a test library of genetic parts of plants (e.g., one or more of test promoters, test terminators, test ribosome binding sites, and the like); and (b) testing the function of the genetic parts of the test library in a platform comprising: (i) a cellular extract prepared from a plant plastid; and (ii) a reaction mixture for transcribing and/or translating an mRNA; and (c) optionally assessing gene expression, and optionally using information obtained from assessing gene expression to modify gene expression in a plant (e.g., by creating a genetically modified plant); and (d) optionally wherein the method is used to is used to characterize and assess the genetic parts and modulate gene expression prior to studying the genetic parts and gene expression in plants.

28. A method comprising: (a) creating a test library of codon-optimized constructs of a gene product; and (b) testing expression of the codon-optimized constructs of the test library in a platform comprising: (i) a cellular extract prepared from a plant plastid; and (ii) a reaction mixture for transcribing and/or translating an mRNA; and (c) optionally using information obtained from testing expression of the codon-optimized constructs to modify expression in a plant (e.g., by creating a genetically modified plant); and (d) optionally wherein the method is used to is used to characterize and assess codon optimization and modulate of gene expression prior to studying codon optimization and modulating of gene expression in plants.

29. A method comprising: (a) creating a test library of constructs expressing several genes; and (b) testing expression of the constructs of the test library in a platform comprising: (i) a cellular extract prepared from a plant plastid; and (ii) a reaction mixture for transcribing and/or translating an mRNA; and (c) optionally using information obtained from testing expression of the construct to modify gene expression in a plant (e.g., by creating a genetically modified plant); and (d) optionally wherein the method is used to is used to provide information to a geneticist regarding the design of multi-gene functions.

30. A method comprising: (a) creating a test library of constructs of a biosynthetic pathway expressing enzymes; and (b) testing the constructs of the test library in a platform comprising: (i) a cellular extract prepared from a plant plastid; and (ii) a reaction mixture for transcribing and/or translating an mRNA; and (c) assessing gene expression, metabolite production, requirement of accessory proteins or other molecules, and/or metabolic parameters; (d) optionally using information obtained from testing the constructs to modify gene expression in a plant (e.g., by creating a genetically modified plant); and (e) optionally wherein the method is used to is used to provide information to a geneticist regarding the design and function of enzyme pathways for example in order to modulate expression levels of the enzymes and testing possible accessory proteins or other molecules required for proper pathway function.

31. A method comprising: (a) creating plant specific sensors comprising genetic circuits that respond to external commands; and (b) testing the sensors in a platform comprising: (i) a cellular extract prepared from a plant plastid; and (ii) a reaction mixture for transcribing and/or translating an mRNA; and (c) assessing sensor response and circuit behavior; and (d) optionally wherein the method is used to is used to provide information to a geneticist regarding the design and function of sensor and genetic circuits.

32. A kit comprising as components: (a) a cell-free extract prepared from a plant plastid (optionally wherein the cellular extract is preserved, such as for example by freeze-drying); and (b) a reaction mixture for transcribing an mRNA from a DNA template and or for translating an mRNA to prepare a polypeptide (optionally wherein the reaction mixture is preserved, such as for example by freeze-drying).

33. The kit of embodiment 30, wherein the plant is Nicotiana tabacum.

34. The kit of embodiment 30 or 31, wherein the reaction mixture comprises one or more components selected from the group consisting of amino acids which optionally may include non-canonical amino acids, NTPs, salts, cofactors, an energy source and optionally an energy source comprising a phosphate group.

35. A method for preparing an extract from plastids of a plant (e.g., isolated intact chloroplasts of a plant), the method comprising: (a) obtaining a plant optionally grown for about 1-8 weeks, for about 2-8 weeks, or for about 4-8 weeks and preferably about 6 weeks; (b) isolating the plastids from the plant; (c) adding a polyol compound (e.g., glycerol) to the isolated plastids (e.g., at a concentration of about 5-15% and preferably at a concentration of about 10%); and (d) lysing the plastids (e.g., by freezing) and separating the extract from the lysed plastids (e.g., separating a soluble extract from the lysed plastids). Optionally, the plastid extract is obtained by lysing cells of the plant and isolating intact plastids from the lysed plant cells. Subsequently, a polyol compound may be added to the isolated intact plastids and the isolated intact plastids then may be lysed. Then, the plastid extract may be separated from the lysed plastids (e.g., via centrifugal separation where the plastid extract soluble and the lysed plastids contain insoluble material such as membrane material and other insoluble material that is precipitated by centrifugation).

Embodiment 36. A maize (e.g., Zea mays L.) plastid cell-free protein synthesis system for in vitro transcription of mRNA and/or translation of polypeptides, comprising: (a) a plastid extract prepared from a maize organ, wherein the organ is optionally not a leaf; (b) a reaction buffer; (c) an RNA polymerase; and (d) a transcription template, wherein the RNA polymerase is capable of transcribing the transcription template to form a translation template and the plastid extract can sustain protein synthesis through a combined transcription/translation reaction.

Embodiment 37. A maize (Zea mays L.) plastid cell-free protein synthesis system for in vitro transcription of mRNA and/or translation of polypeptides, comprising: (a) a plastid extract prepared from a maize organ, wherein the maize has been grown for 1-8 weeks; (b) a reaction buffer; (c) an RNA polymerase; and (d) a transcription template, wherein the RNA polymerase is capable of transcribing the transcription template to form a translation template and the plastid extract can sustain protein synthesis through a combined transcription/translation reaction.

Embodiment 38. The system of claim 37, wherein the maize has been grown for about 2 weeks.

Embodiment 39. The system of claim 37 or 38, wherein the maize has been grown in about 8 hours of light and about 16 hours of darkness.

Embodiment 40. The system of any one of claims 37-39, wherein the maize has been grown in about 10 hours of darkness immediately before the extract is prepared.

Embodiment 41. The system of any one of claims 36-40, wherein the plastid extract is prepared from a plant that has been engineered to express T7 RNA polymerase in the plastid or in the nucleus.

Embodiment 42. The system of claim 41, wherein the T7 RNA polymerase is targeted to the plastid.

Embodiment 43. The system of any one of claims 36-42, wherein the maize is engineered to be deficient in a negative effector for cell-free protein synthesis (CFPS), or combined transcription and translation.

Embodiment 44. A method for in vitro transcription of mRNA and/or translation of a polypeptide, comprising: reacting a maize plastid cell-free protein synthesis system with a linear or plasmid template for transcription of the mRNA and/or a template for translation of the polypeptide; monomers for synthesis of the mRNA and/or the polypeptide; co-factors, enzymes and/or other reagents necessary for the transcription and/or translation; and magnesium at a concentration of from about 3 mM to about 20 mM (or from about 4 mM to about 12 mM, or from about 6 mM to about 10 mM).

Embodiment 45. The method of claim 44, wherein the maize plastid extract is the cell-free protein synthesis system of any one of claims 36-44.

Embodiment 46. The method of claim 44, wherein the monomers comprise amino acids at a concentration of between about 0.5 to 4 mM, and preferably at a concentration of about 2 mM.

Embodiment 47. The method of claim 44, wherein the reaction mixture further comprises potassium at a concentration of about 0-200 mM, and preferably at a concentration of about 100 mM.

Embodiment 48. The method of claim 44, wherein the reaction mixture further comprises salts at a total concentration of between about 50-400 mM.

Embodiment 49. The method of claim 44, wherein the method is performed using a liquid handling robot.

Embodiment 50. The method of any one of claims 44-49, wherein the template encodes a test library of genetic components of maize.

Embodiment 51. The method of claim 50, further comprising testing the function of the genetic components of the test library in the maize plastid cell-free protein synthesis system and assessing gene expression in the system.

Embodiment 52. The method of claim 50, wherein the method is used to characterize and assess the genetic parts or components and fine-tune gene expression prior to studying the genetic components and gene expression in maize.

Embodiment 53. The method of claim 50, wherein the genetic components comprise at least two selected from the group consisting of promoters, terminators, ribosome binding sites, and maize genes.

Embodiment 54. The method of claim 50, wherein the genetic components are genetic clusters.

Embodiment 55. The method of any one of claims 44-49, wherein the template encodes a test library of codon-optimized constructs of a gene product.

Embodiment 56. The method of claim 55, further comprising testing expression of the codon-optimized constructs of the test library in the maize plastid cell-free protein synthesis system and using information obtained from testing expression of the codon-optimized constructs to modify expression in a maize plant.

Embodiment 57. The method of claim 55, wherein the method is used to characterize and assess codon optimization and fine-tune of gene expression prior to studying codon optimization and fine-tuning of gene expression in plants.

Embodiment 58. The method of any one of claims 44-49, wherein the template encodes a test library of constructs expressing several genes.

Embodiment 59. The method of claim 58, further comprising testing expression of the constructs of the test library in the maize plastid cell-free protein synthesis system and using information obtained from testing expression of the construct to modify gene expression in a plant.

Embodiment 60. The method of claim 58, wherein the method is used to is used to provide information to a geneticist regarding the design of multi gene functions.

Embodiment 61. The method of any one of claims 44-49, wherein the template encodes a test library of constructs of a biosynthetic pathway expressing enzymes.

Embodiment 62. The method of claim 61, further comprising testing the constructs of the test library in the maize plastid cell-free protein synthesis system and assessing gene expression, metabolite production, requirement of accessory proteins or other molecules, and/or metabolic parameters.

Embodiment 63. The method of claim 61, wherein the method is used to aid design of multi gene functions; and

Embodiment 64. The method of claim 61, wherein the method is used to aid the design and function of enzyme pathways for example in order to fine-tune expression levels of the enzymes and testing possible accessory proteins or other molecules required for proper pathway function.

Embodiment 65. The method of any one of claims 44-49, wherein the template encodes plant specific sensors comprising genetic circuits that respond to external commands.

Embodiment 66. The method of claim 65, further comprising testing the sensors in the maize plastid cell-free protein synthesis system and assessing sensor response and circuit behavior.

Embodiment 67. The method of claim 65, wherein the method is used to aid the design and function of sensor and genetic circuits.

### EXAMPLES

The following Examples are illustrative and should not be interpreted to limit the scope of the claimed subject matter.

### Example 1—A Novel Combined Transcription and Translation Platform Derived From Plant Chloroplasts

Abstract

The current state of the art in transformation of plant chloroplasts is a laborious, low-throughput method that takes approximately one year to produce a stably transgenic plant. As a result, developments in plant biotechnology and our understanding of the basic biology of plants have lagged far behind what we have been able to achieve with mammalian or prokaryotic biology, in spite of the fact that plants are considered to be the source of a rich potential for pharmaceutically and technologically relevant natural products. Here we present a chloroplast cell-free protein synthesis platform derived from Nicotiana tabacum that would allow for high-throughput screening of genetic parts and allow for targeted design before plant transformation. This is the first ever—to our knowledge—combined transcription and translation system from plant chloroplasts. We have developed a series of protocols comprising plant growth conditions, a harvest and lysis protocol, and optimized CFPS reaction mixture that can be used to generate chloroplast extract for prototyping genetic parts. The optimized system is capable of producing up to ˜30 μg/mL of luciferase reporter protein, which can be detected by plate reader and affords enough dynamic range to begin to determine nuances in genetic parts libraries.

Applications

Applications for the disclosed technology include, but are not limited to: (i) In vitro screening of enzyme pathway variants for plant metabolic engineering; (ii) Pre-screening DNA designs with chloroplast CFPS allows for a dramatic reduction in iteration time for research and development; (iii) Synthetic biology applications; (iv) Fundamental studies of the chloroplast translation apparatus and harnessing translation machinery for novel functions; (v) High-throughput interrogation and characterization of organismal genetic parts (promoters, terminators, ribosome binding sites, etc.); and (vi) Agriculture and Crop Science.

Advantages

Applications for the disclosed technology include but are not limited to the following aspects.

The current state of the art in chloroplast transformation takes approximately one year to produce transformants, meaning that design iterations occur annually. Chloroplast transformation is a laborious process that involves bombardment of plant tissue with gold particles loaded with DNA. This method is not sufficiently high-throughput for prototyping genetic parts. The disclosed technology can be utilized to significantly curtail the amount of time needed to perform multiple genetic tests. For example, instead of performing 10 genetic tests in 9 month, one could perform 10,000 tests in a week.

While it is possible to use plant cell culture to study candidates for transformation into plants and some plant cell lines such as BY-2 cell lines contain chloroplasts, their chloroplasts are undifferentiated, and therefore less suitable to mimic cellular processes of a fully developed functional chloroplast. Similarly, wheat germ extract could be used to prototype nuclear modifications. However, the chloroplast is a more desirable location to stably express exogenous DNA because there is no silencing of organelle DNA. In addition, several genes can be expressed from an operon and the maternal inheritance of this subcellular compartment prevents exogenous gene escape through pollen.

Brief Summary of Technology

Cell-free protein synthesis (CFPS) is fueling numerous applications as a powerful in vitro expression system. Yet, there is little available for in vitro genetic prototyping for plant biologists. We have applied our expertise in cell-free systems to the existing work on in vitro translation to develop the first ever combined transcription-translation cell free platform derived from chloroplasts. We chose Nicotiana tabacum for its large leaf size and rapid growth, as well as well as the fact that it grows readily in an indoor setting. Currently, we have demonstrated the ability to transcribe plasmid DNA in tobacco chloroplast cell-free extracts. Then, we next achieved combined transcription and translation by improving the plants' growth conditions and selecting the BY genetic background. Next, we sought to improve yields by optimizing our harvest conditions. Initially, we collected leaves at 4 weeks post-germination, the earliest reasonable time based on tissue mass available from the plants. As a follow-up, we allowed the plants to recover for two weeks and harvested leaves again at 6 weeks. We found that chloroplast extract from 6-week-old plants is over six-fold more productive in CFPS than that from 4-week-old plants. Once we determined the optimal growth conditions, we sought to improve our harvest and lysis protocol. Surprisingly, we found that freezing chloroplasts resuspended in lysis buffer with 10% glycerol had a dramatically positive effect (which was surprising since this has not been done before). Specifically, results show that addition of glycerol prior to freezing improves yields 30-fold. Finally, we sought to optimize our cell-free reactions in a two-pronged approach. First we surveyed a number of plasmids with different 5′-UTR ad 3′-UTR to boost yields and validate our hypothesis that the chloroplast cell-free system could be used to assess and rank different genetic parts. Further, results from this library have shown that the 5′-UTR has shown to be more important than the 3′-UTR for high yields. Next, we optimized the reaction environment by varying the levels of small molecules, enzymes, and crowding in our reactions. We found that high concentrations of amino acids were important to obtain high yields. Currently our batch reactions produce stable, active luciferase or GFP for about three hours and can be used to rank 5′-UTRs. We expect that this technology will be highly desirable to large biotechnology companies that seek to prototype on a more rapid timescale.

Problems Solved

Problems solved by the disclosed technology include but are not limited to the following aspects. Technological developments in plant biology have historically been slow, in spite of the fact that our early understanding of genetics was pioneered in plants. Previously, prototyping in BY-2 cell culture has informed transformation into the nuclear genome, though this is not necessarily a high-throughput process and nuclear transformation is susceptible to gene silencing. Transformation into the chloroplast is more generationally stable, but is highly time-consuming, requiring one year to produce transformants. There is also no known way of pre-screening candidates for transformation in a chloroplast-like environment. As a result, the current compromise is to devote a staggering number of person-hours to development of a small number of candidates or to prototype in a nuclear context.

Commercial Aspects

Commercial aspects of the disclosed technology include but are not limited to the following. Previous researchers have sought to produce chloroplast extracts for in vitro translation, but this has proved challenging, likely due to the fact that freeze/thaw and handling can rupture chloroplasts. Our method includes addition of 10% glycerol lysis buffer prior to freezing, which ensures active extract. It is at present unclear if this prevents lysis entirely or merely protects chloroplast proteins and structures through the thawing process.

Plant biotechnology companies seek to increase their design-build-test cycles to increase revenue. Our platform could be used to screen thousands of genetic parts in the amount of time that it would take to produce one round of chloroplast transformants. Currently, the system is able to produce up to 28 μg/mL of active luciferase, which we have shown to be enough dynamic range to rank a small library of 5′-UTR and 3′-UTR variants. This technology could be combined with the well-understood practice of tobacco chloroplast transformation to produce large amounts of medically or agriculturally relevant compounds. Alternatively, our methods could be applied to chloroplasts derived from therapeutically or agriculturally relevant species to better prototype in more distantly related plant species such as grasses, trees, or algae. Our initial discovery has opened up the possibility to manipulate levels of natural products in many different areas of plant biotechnology.

This system is the first ever chloroplast-derived CFPS system capable of transcription and translation and is more high-yielding than previously reported translation-only systems. While other plant-derived systems have been published (wheat germ extract and BY-2 extract), these systems are not suited to prototyping for the chloroplast, which is a more stable transformation cite than the nucleus. This system will enable technological discovery by generating a method for rapid prototyping in a plant context.

Results

Aspects of this Example are further described in the figures that accompany this application and the corresponding figure legends.

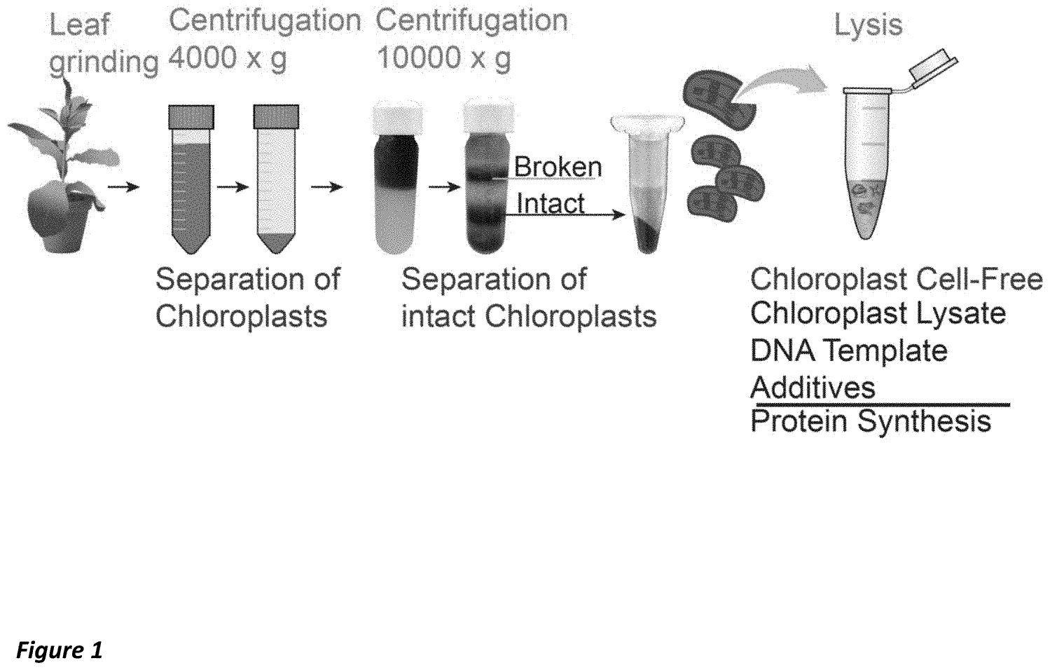

FIG. 1 provides an overview of the isolation of intact chloroplasts from plant material and preparation of an extract from the isolated chloroplasts for use in cell-free transcription and/or translation. In a first step, plant material, such as leaf material is ground and chloroplasts are separated from the ground plant material via low speed centrifugation (e.g., 4000×g). Next, the separated chloroplasts are overlaid on a gradient and subjected to higher speed centrifugation (10000×g) to separate broken chloroplasts from intact chloroplasts. Intact chloroplasts are isolated and subjected to lysis (e.g., via freezing) to prepare an extract which may be utilized as part of a cell-free protein synthesis (CFPS) reaction mixture comprising the extract, a DNA expression construct, and additional additives for protein synthesis.

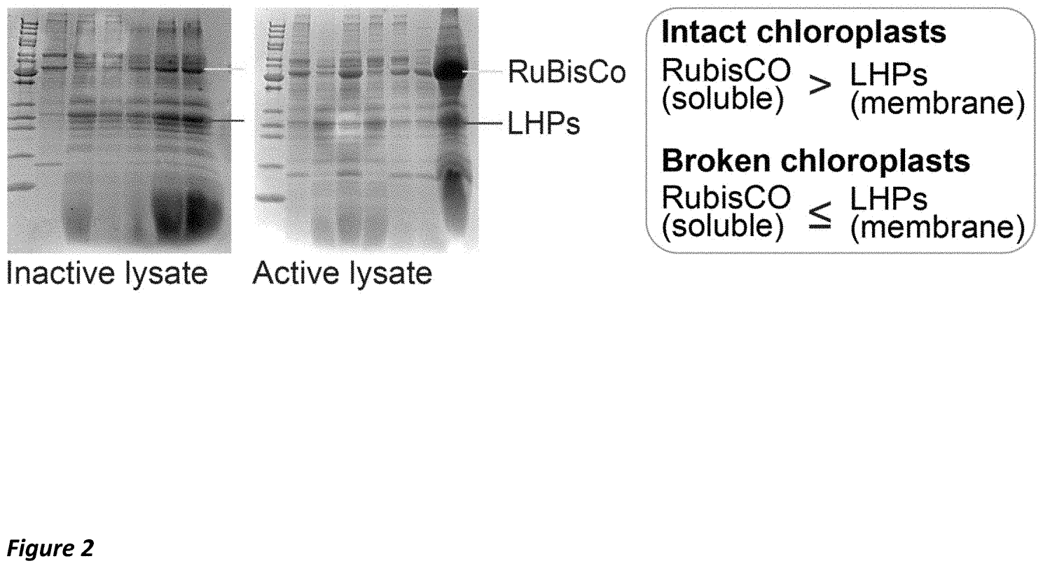

The activity of a lysate in cell-free protein synthesis reactions is correlated with the amount of intact chloroplasts utilized to prepare the lysate. FIG. 2 illustrates the relative protein concentration of ribulose-1,5-bisphosphate carboxylase/oxygenase (Rubisco), an enzyme present in chloroplasts, versus the concentration of membrane proteins in a extract can be utilized as an indicator of intact chloroplasts versus broken chloroplasts in plant material.

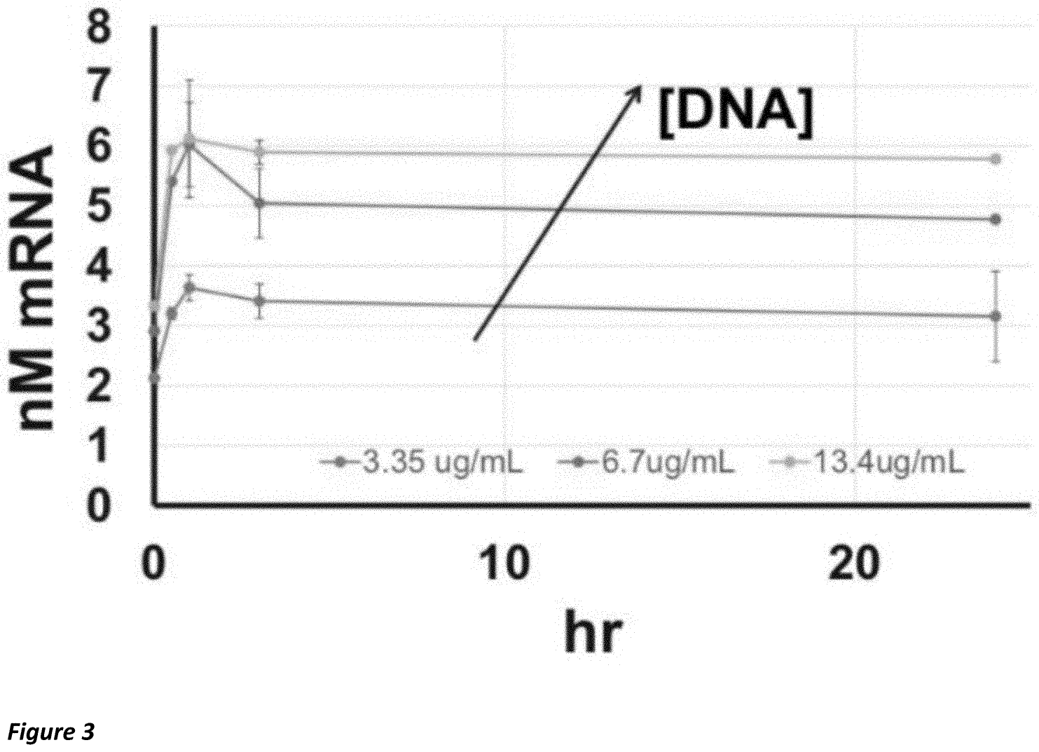

Chloroplast extracts can be utilized in cell-free systems to express mRNA. FIG. 3 illustrates the production of mRNA in a cell-free system comprising a chloroplast extract as a function of the concentration of a DNA template for mRNA present in the system. As illustrated, mRNA production (concentration) was correlated with the concentration of DNA template added to the cell-free system.

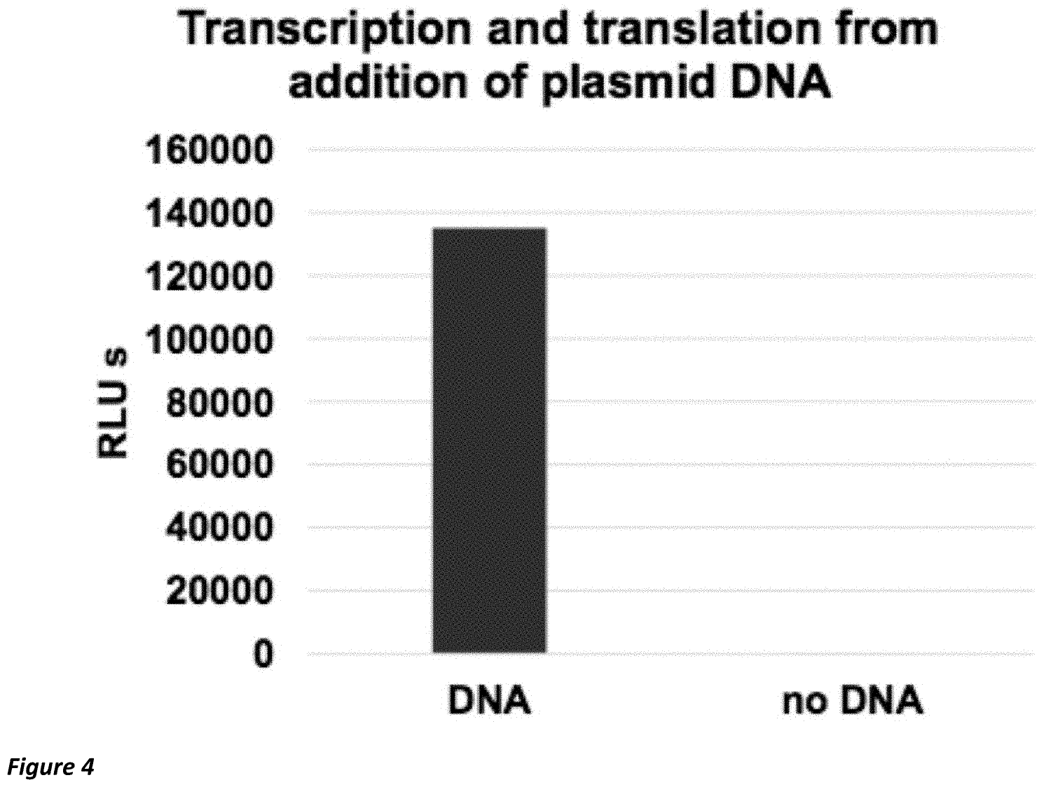

Chloroplast extracts also can be utilized to express a protein. FIG. 4 illustrates the results of experiments in which a template DNA encoding a fluorescent target protein was transcribed and translated in cell-free systems comprising a chloroplast extract. Protein production was only observed in the presence of template DNA.

Having established a strategy to prepare chloroplast extracts that were competent for transcription and translation, we set out to improve protein yields in our cell-free protein synthesis (CFPS) systems to ˜1-5 μg/mL.

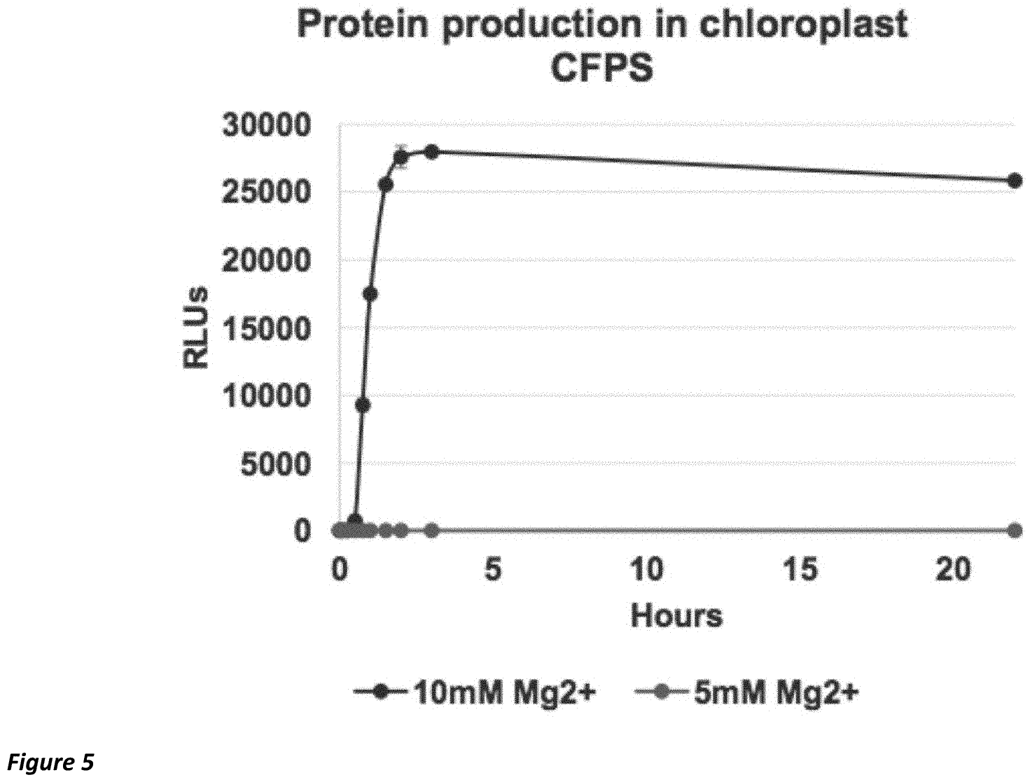

First, because divalent cations are known to be required in CFPS systems, we assessed protein production in our chloroplast extract CFPS system in the presence of the divalent cation Mg²⁺. FIG. 5 illustrates that divalent cations such as Mg²⁺ are required for efficient protein production in a cell-free system comprising a chloroplast extract in which a template DNA encoding a fluorescent target protein was transcribed and translated.

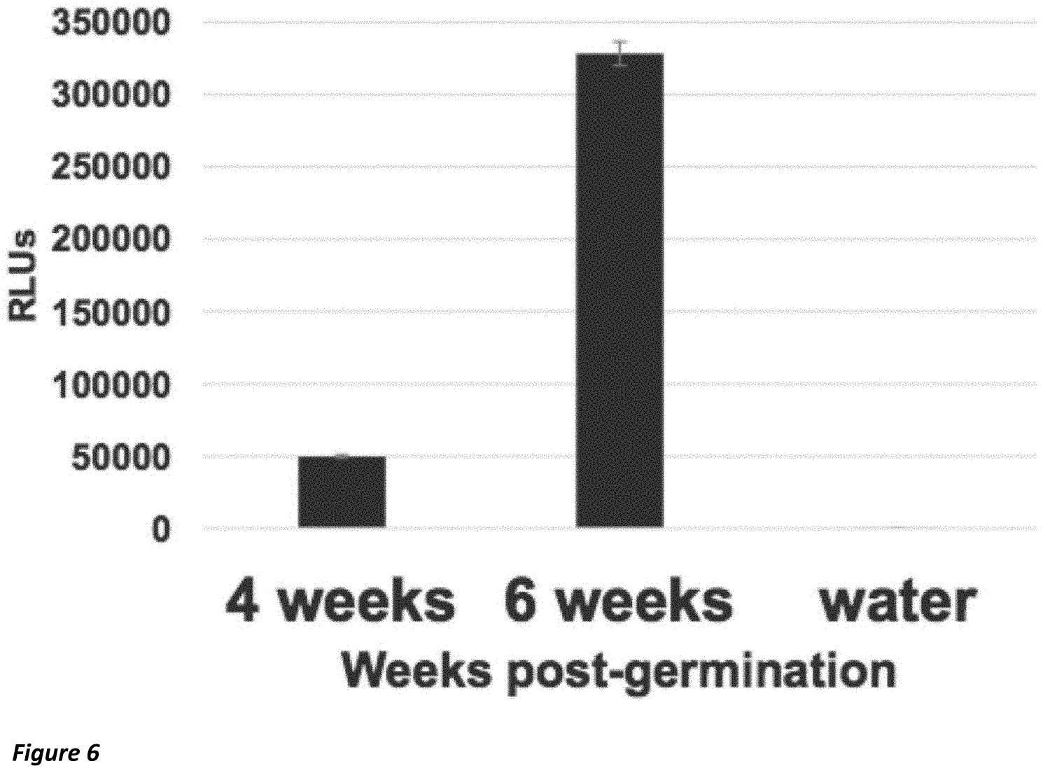

We also assessed whether we could obtain higher efficiency of protein production in our CFPS system by using extracts prepared from plants that had been grown for different periods of time. FIG. 6 illustrates that chloroplast extracts prepared from chloroplasts isolated from plants that were grown at six (6) weeks versus four (4) weeks exhibit better performance regarding protein production in a cell-free free system. Chloroplasts from 6 week old plants are over six-fold more productive in CFPS than those from 4 week old plants

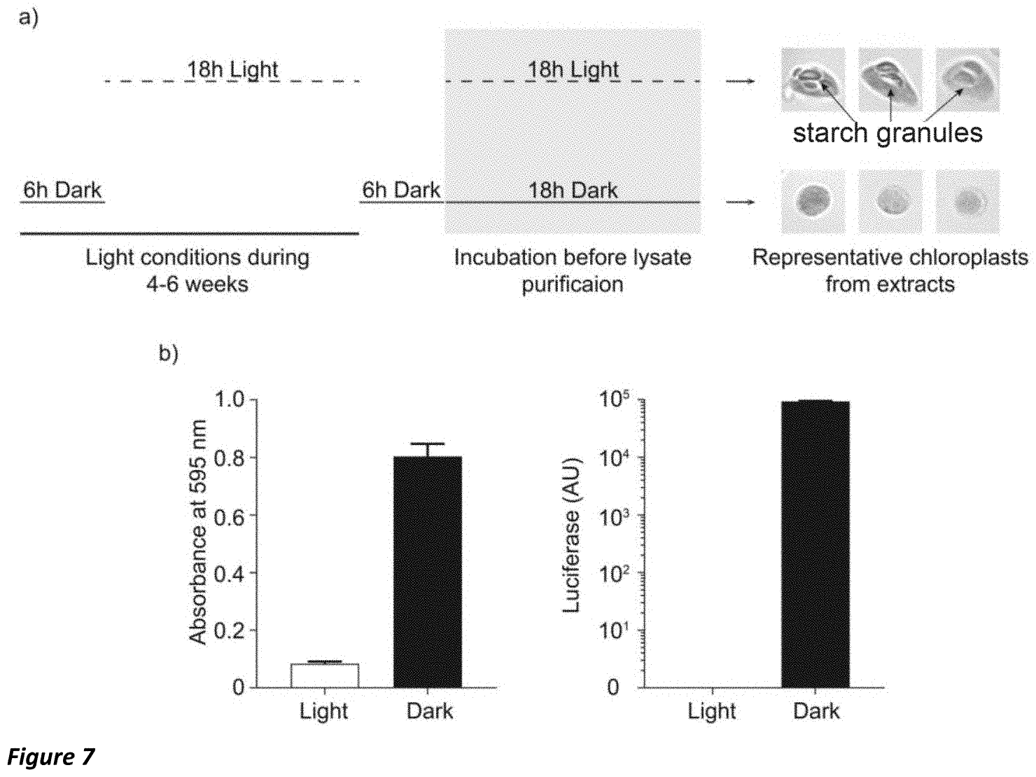

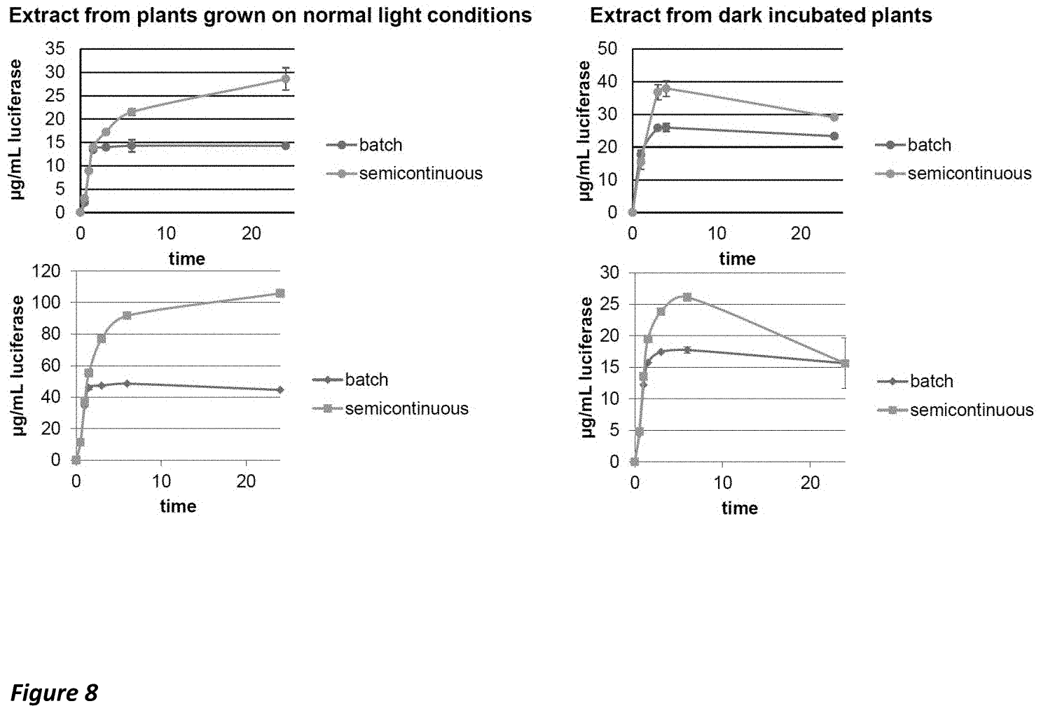

Because exposure to light is known to increase the production of starch in chloroplasts which may make chloroplasts more susceptible to membrane breakage during isolation, we assessed whether we could obtain extracts that exhibited higher production in our CFPG system by using extracts prepared from plants that had been exposed to darkness prior to our isolating chloroplasts from the plants and preparing extracts. FIG. 7 illustrates a method to increase lysate activity related to total protein content by dark incubation of plants before extract preparation. After 6-24 h of dark incubation starch granules are reduced in the chloroplasts as observed by light microscopic images which results higher total protein content as measured with Bradford assay and lysate productivity as measured by expressed luciferase in the chloroplast cell-free system. FIG. 8 illustrates the results of semi-continuous and batch reaction of extracts from light/dark incubated plants. Semi-continuous reaction conditions were observed to increase protein output independent of light/dark treatment, although substrate replenishment appeared different in “light” versus “dark” extracts. This might be the result of higher activity of proteases in dark incubated plants or that dark exposure might lead to different metabolic/translational states. Dark and light exposed chloroplast extracts could have different applications.

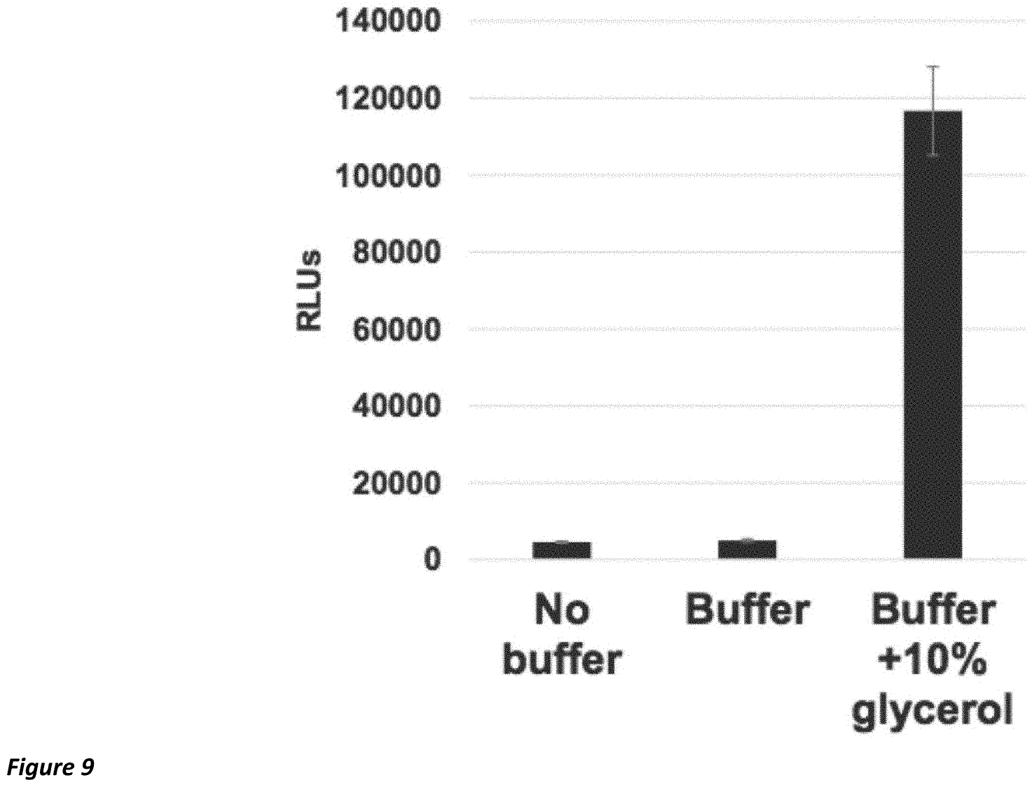

Because lysing may disrupt some of the supramolecular structures of components utilized in transcription/translation, we tested whether we could add an agent that may stabilize supramolecular structures after lysis. As such, we assessed whether the addition of a polyol such as glycerol to our isolated chloroplasts prior to lysis by freezing could improve productivity of the lysates. As illustrated in FIG. 9, protein production is improved in our chloroplast CFPS system when a polyol such as glycerol is added, and addition of glycerol prior to lysis by freezing the chloroplasts improved yields by 30-fold.

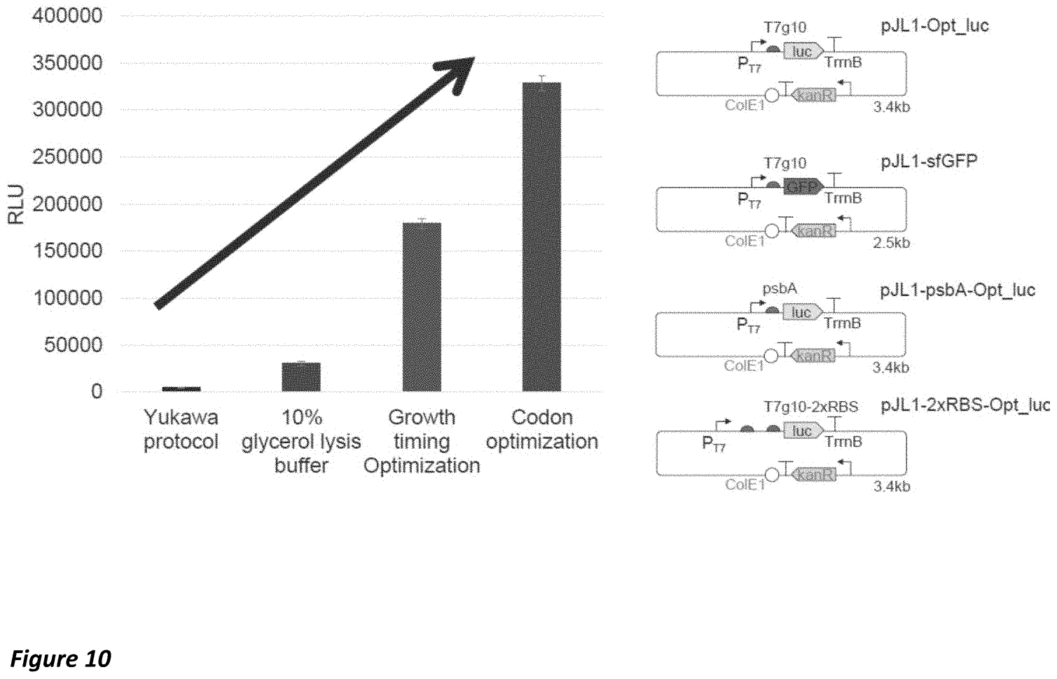

We also assessed whether we could obtain additive improvement in protein productivity by utilizing all of the individual conditions that were observed to improve protein in combination. FIG. 10 illustrates the results of combined optimization on protein production in a cell-free system comprising a chloroplast extract, including addition of a polyol, timing of growth in plants from which the chloroplast extract is prepared, codon optimization for expression in the plants from which the chloroplast extract is prepared.

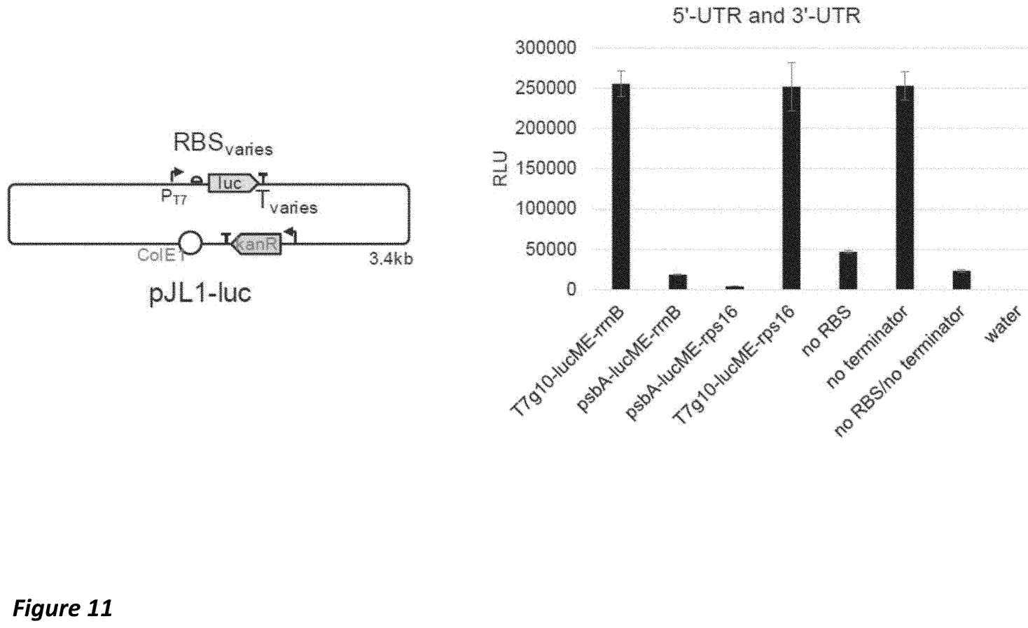

We next assessed whether we could modify elements that are present in the 5′-UTR and 3′-UTR of our DNA expression template in order to modulate protein production. FIG. 11 illustrates that protein expression is influenced by modifications in the 5′-UTR, such as modifications in the ribosome binding site (RBSD) and in the 3′-UTR, such as modifications in the transcription terminator.

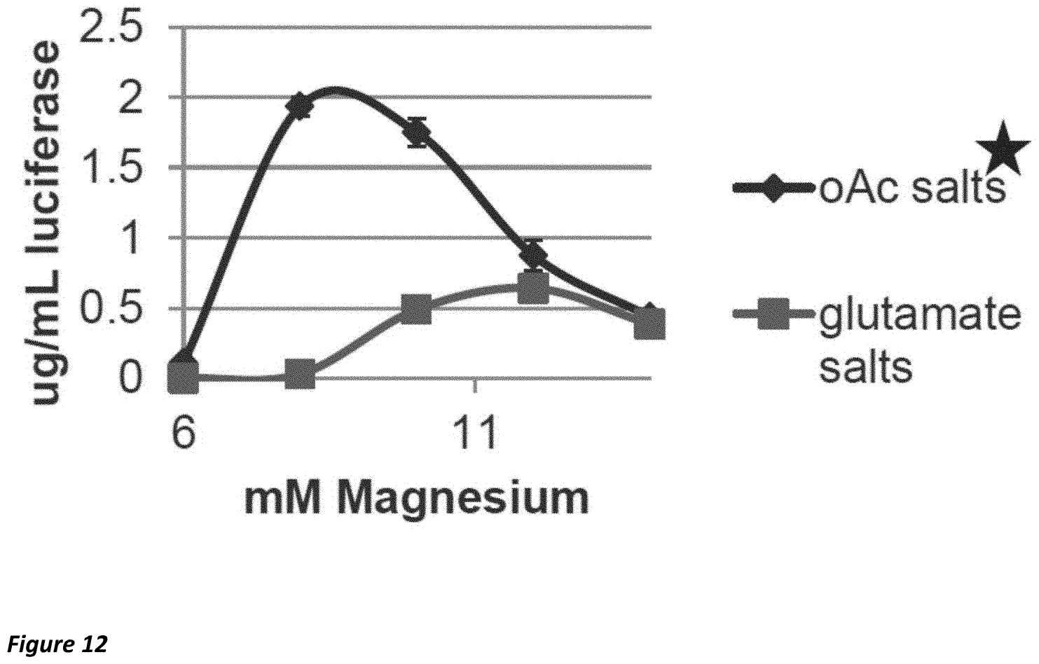

We next tested various salt, buffer, and pH conditions to determine whether we could optimize yields in our chloroplast extract CFPS system. FIG. 12 illustrates the effect of magnesium acetate versus magnesium glutamate on protein production in a cell-free system comprising a chloroplast extract. Magnesium acetate at a concentration between about 6-11 mM (e.g., about 8-10 mM) was observed to optimize protein production.

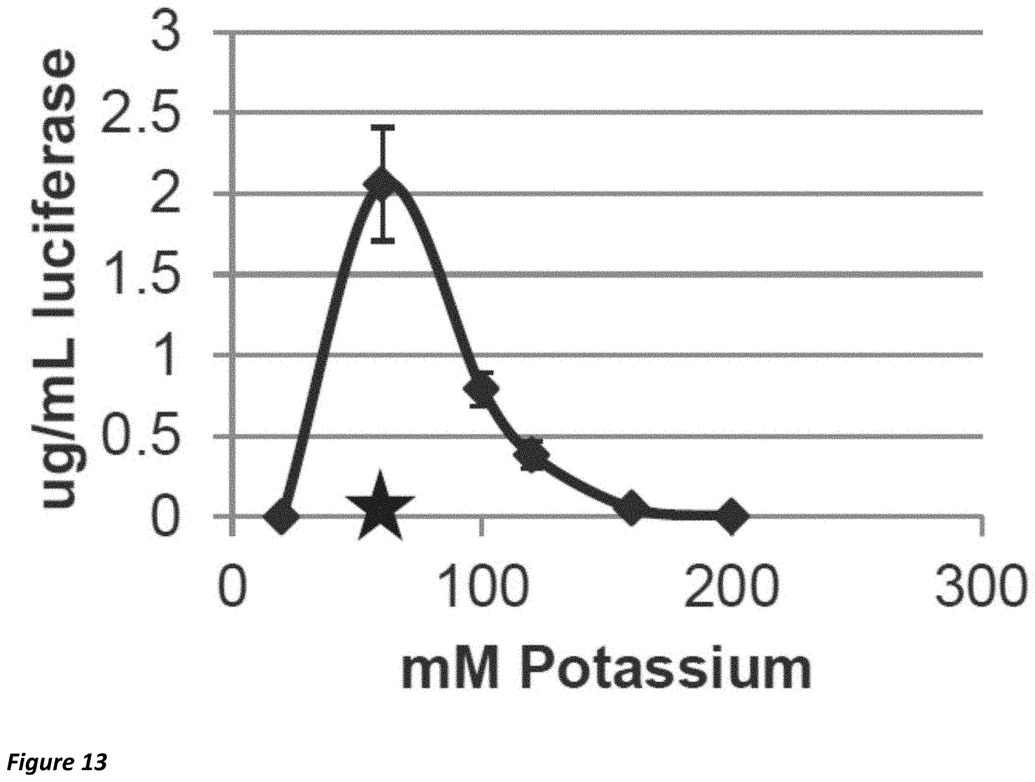

FIG. 13 illustrates the effect of adding potassium on protein production in a cell-free system comprising a chloroplast extract. A concentration of potassium of about 50-80 mM (e.g., about 55-70 mM) was observed to optimize protein production.

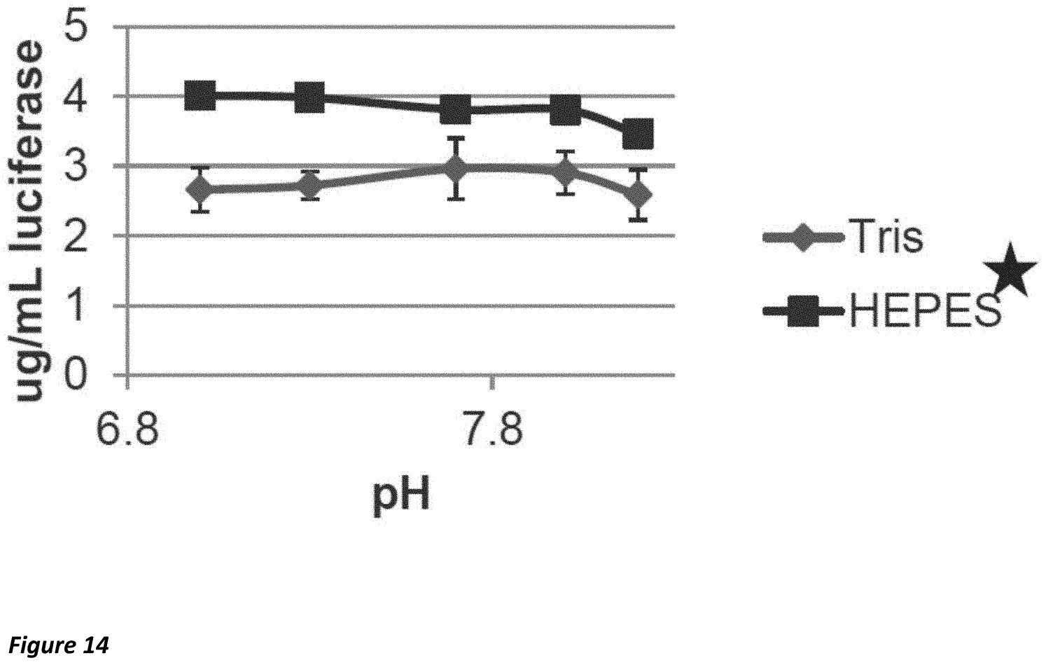

FIG. 14 illustrates the effect of pH in the presence of a HEPES buffered system versus a Tris buffered system on protein production in a cell-free system comprising a chloroplast extract. HEPES was observed to perform better than Tris in optimizing protein production and a pH of less than about 7.8 was observed to be optimal for protein production (e.g., less than about 7.7, 7.6, or 7.5 and/or higher than about 6.8).

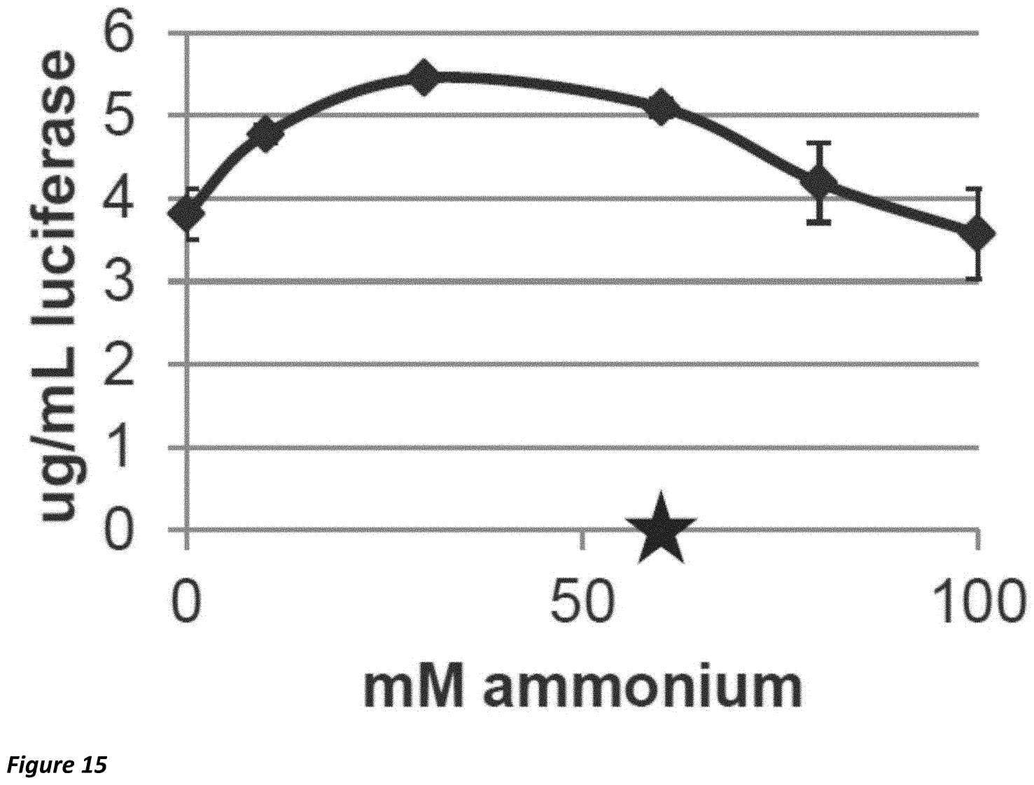

FIG. 15 illustrates the effect of adding ammonium on protein production in a cell-free system comprising a chloroplast extract. Protein production was optimal when ammonium was present at a concentration of about 20-60 mM (e.g., about 30-50 mM).

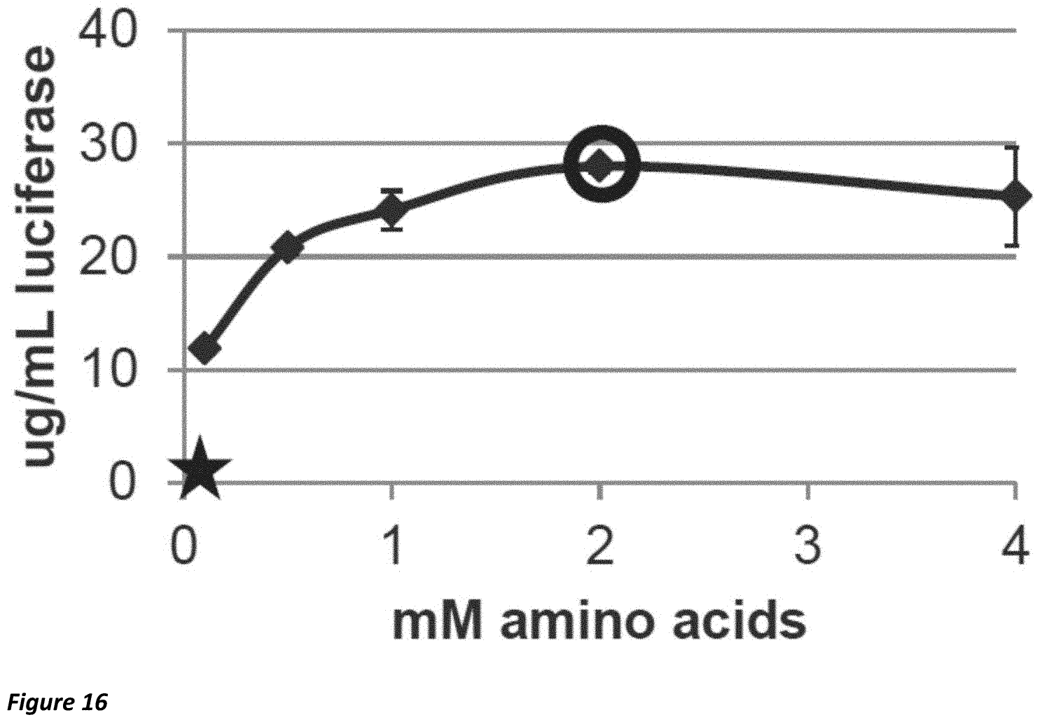

FIG. 16 illustrates the effect of amino acid concentration on protein production in a cell-free system comprising a chloroplast extract. Protein production was optimal when amino acids were present at a concentration of about 1-3 mM (e.g., about 2 mM).

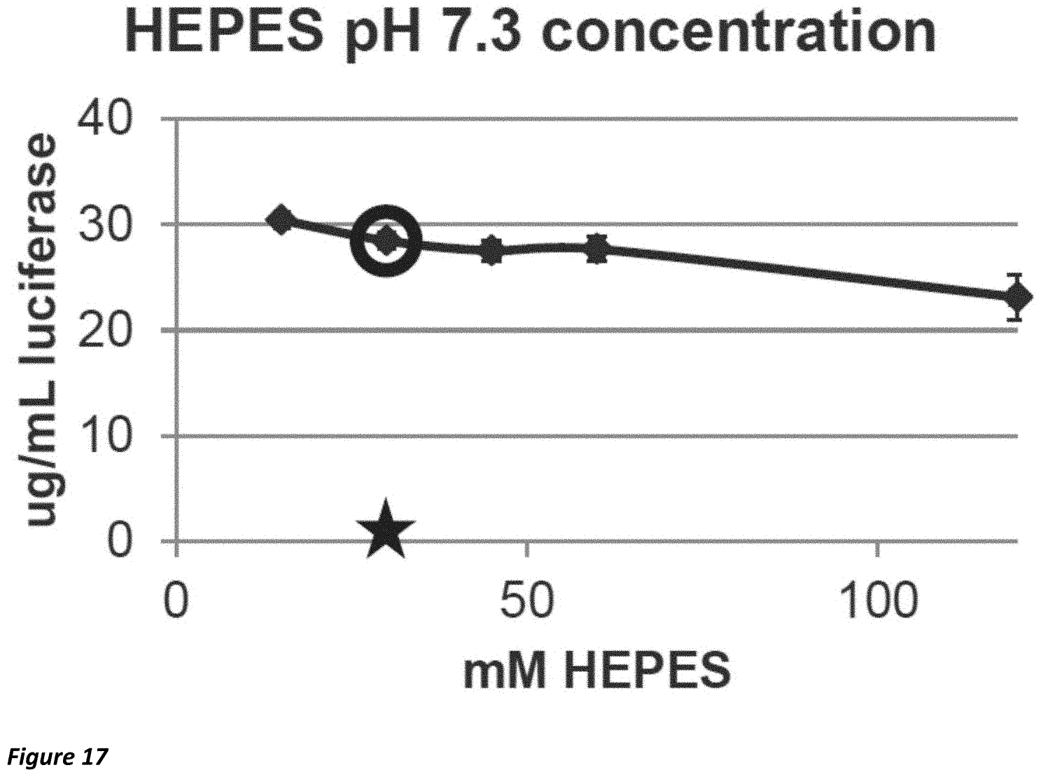

FIG. 17 illustrates the effect of HEPES concentration at a pH of 7.3 on protein production in a cell-free system comprising a chloroplast extract. Protein production was optimal when HEPES was present at a concentration of about 20-50 mM.

In summary, we observed that optimizing the pH and the concentration of ammonium, HEPES, and amino acids, we could increase the protein yield of an extract from ˜7 μg/ML to 30 μg/mL.

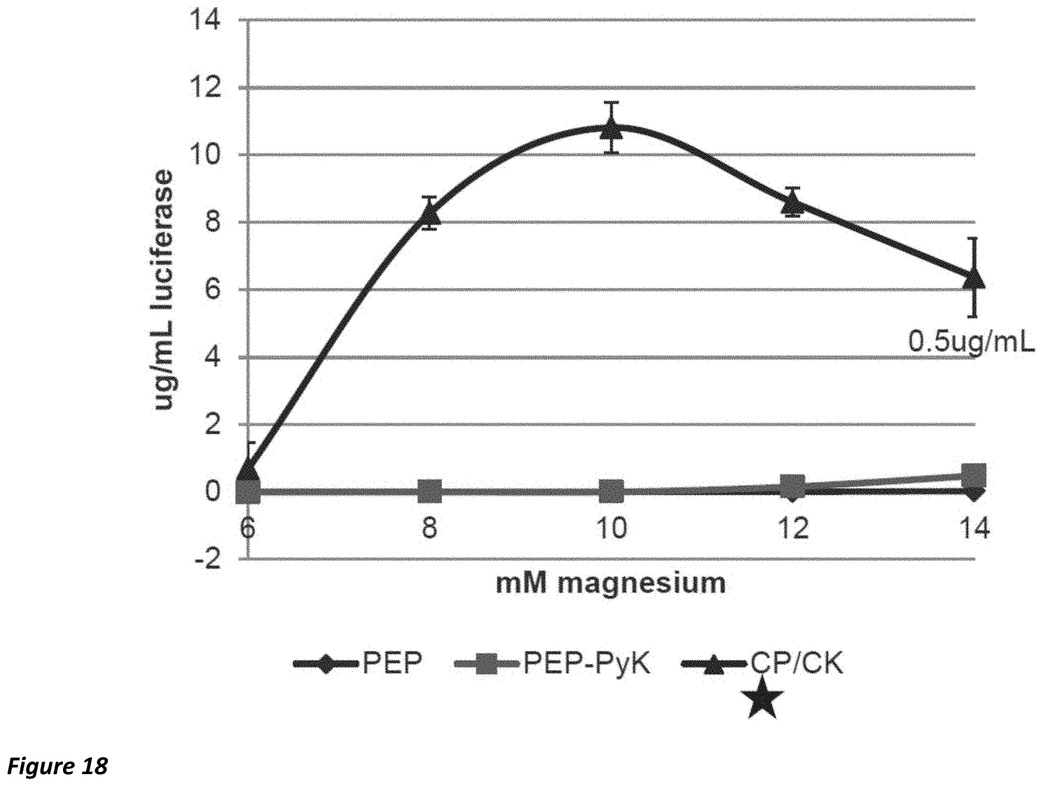

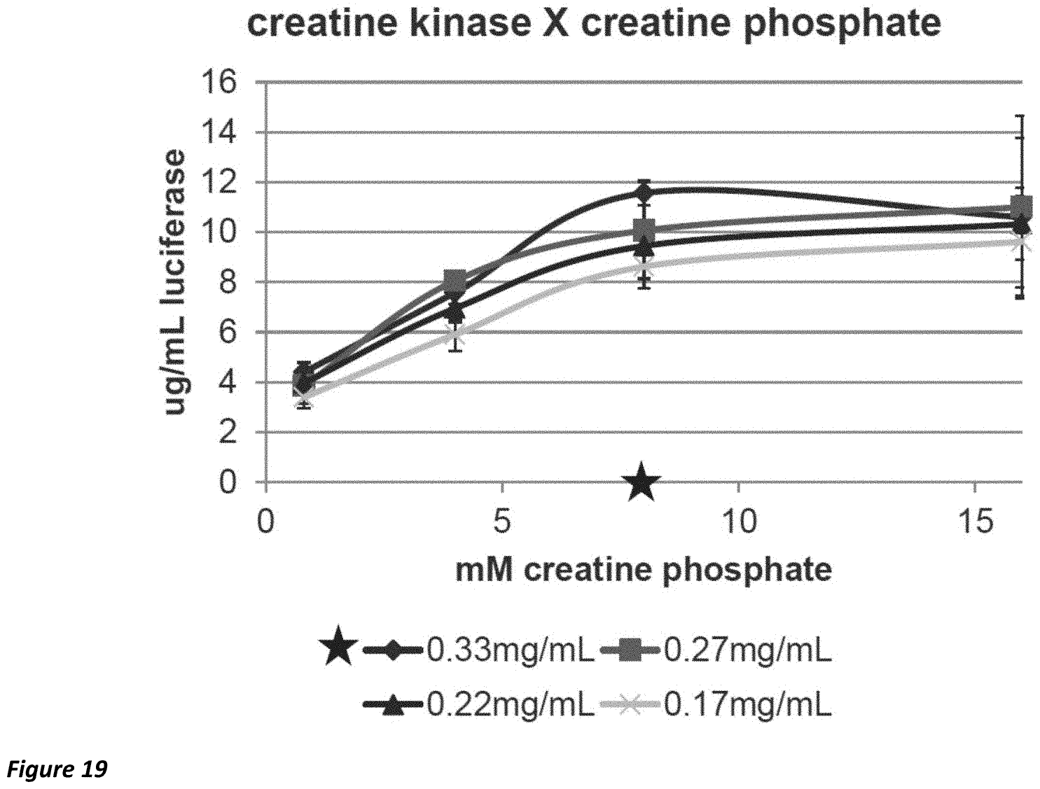

We also assessed which energy regeneration systems resulted in highest efficiency in our chloroplast extract CFPS system. FIG. 18 illustrates the effect of different energy regeneration systems on protein production in a cell-free system comprising a chloroplast extract, including a phosphoenolpyruvate (PEP) system, a PEP/pyruvate kinase (PyK) system, and a creatine phosphate (CP)/creatine kinase (CK) system. Protein production required the CP/CK system. FIG. 19 illustrates the effect of different concentrations of a creatine phosphate (CP)/creatine kinase (CK) energy regeneration system on protein production in a cell-free system comprising a chloroplast extract. A concentration of about 0.27-0.33 mg/mL was observed to optimize protein production.

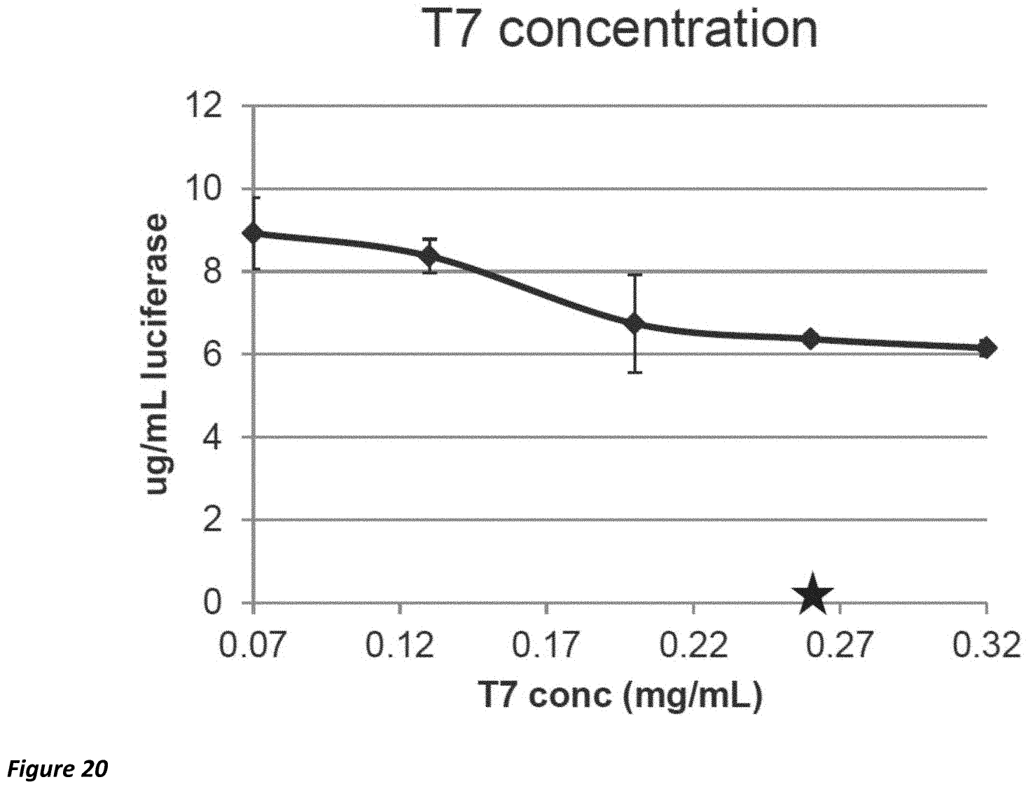

We also assessed whether we could improve protein production by optimizing the concentration of the DNA-dependent RNA polymerase from T7 (T7 RNAP) in our chloroplast extract CFPS system. FIG. 20 illustrates the effect of different concentration of T7 RNAP on protein production in a cell-free system comprising a chloroplast extract.

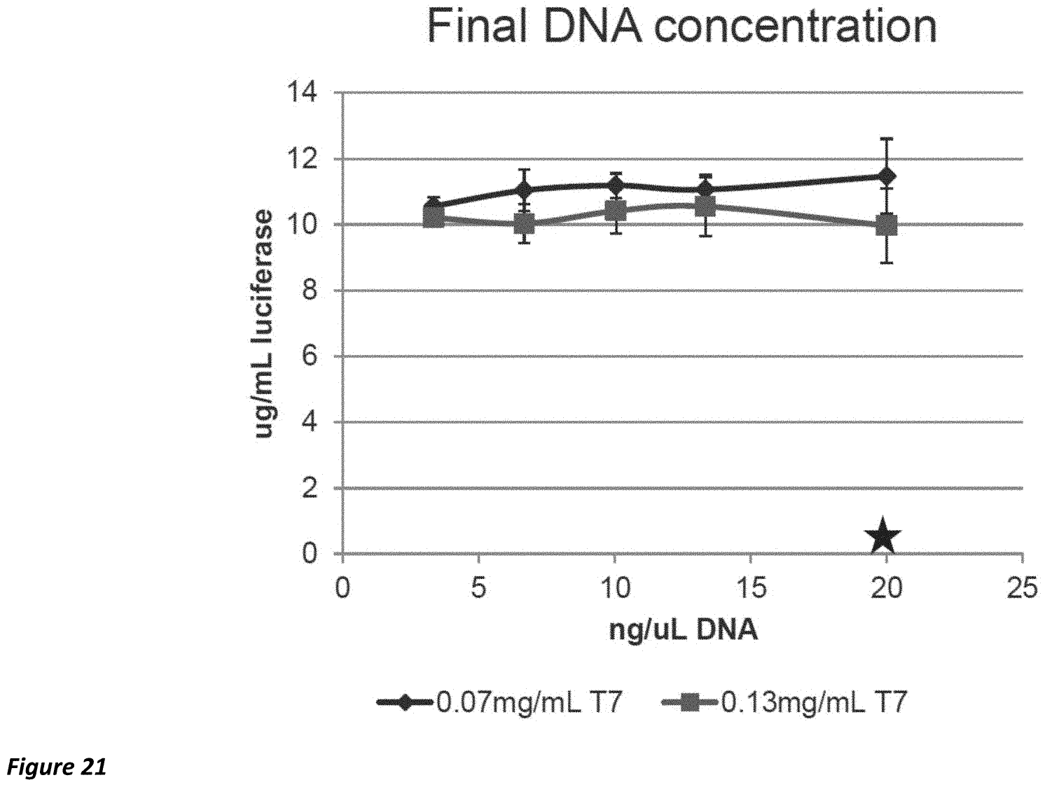

We also assessed whether we could improve protein production by optimizing the concentration of DNA template in our chloroplast extract CFPS system. FIG. 21 illustrates the effect of final DNA template concentration at two different concentrations of T7 polymerase on protein production in a cell-free system comprising a chloroplast extract.

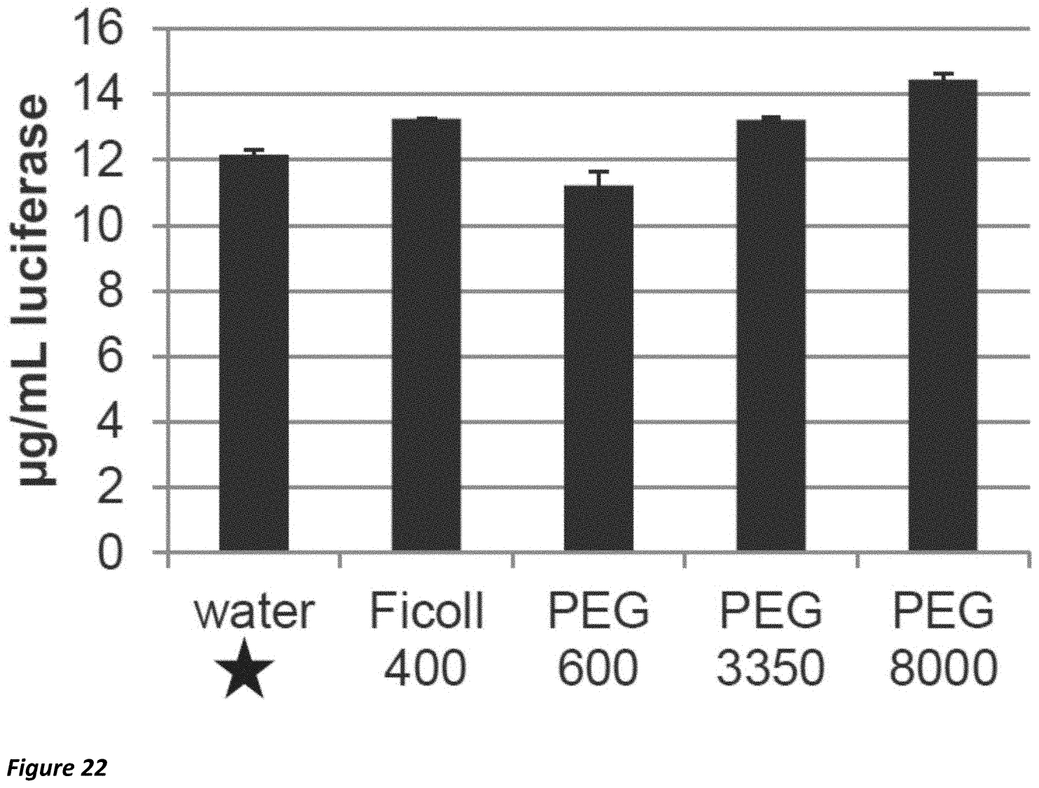

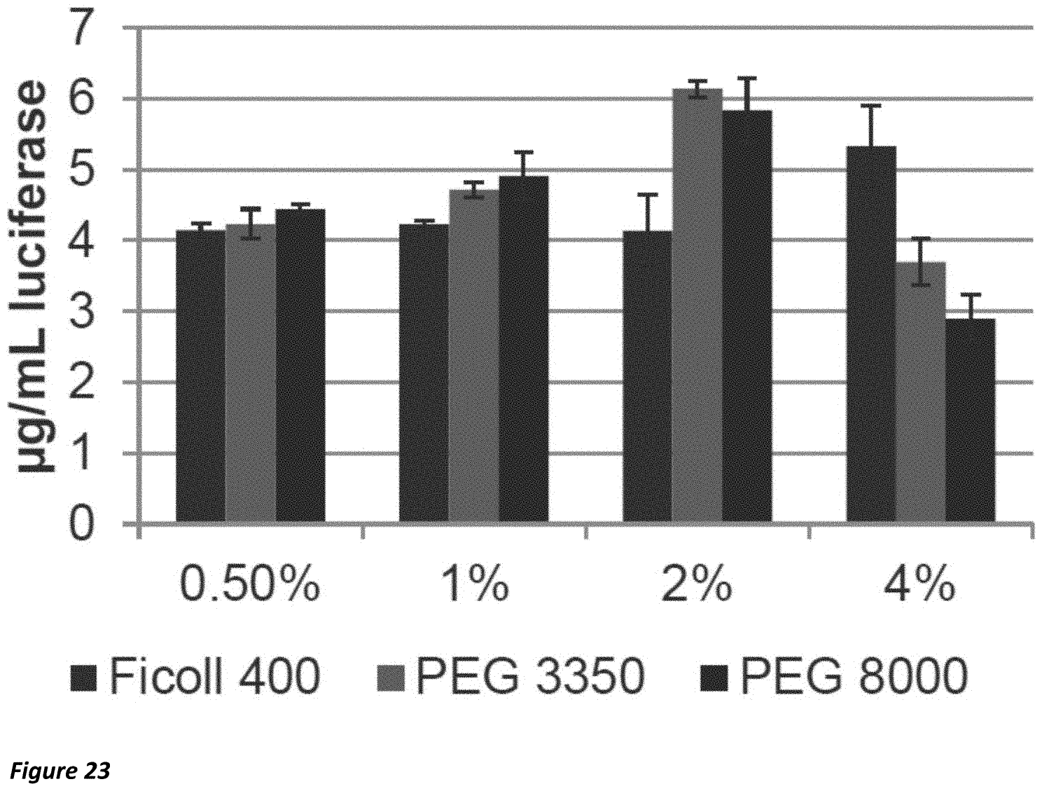

We also assessed whether we could improve protein production by adding macromolecular crowding agents to our chloroplast extract CFPS system. FIG. 22 illustrates the effect of different macromolecular crowding agents on protein production in a cell-free system comprising a chloroplast extract, including Ficoll 400, PEG 600, PEG 3350, and FEG 8000. FIG. 23 illustrates the effect of different macromolecular crowding agents at different concentrations on protein production in a cell-free system comprising a chloroplast extract, including Ficoll 400, PEG 3350, and PEG 8000 at one of 0.50%, 1%, 2%, or 4%.

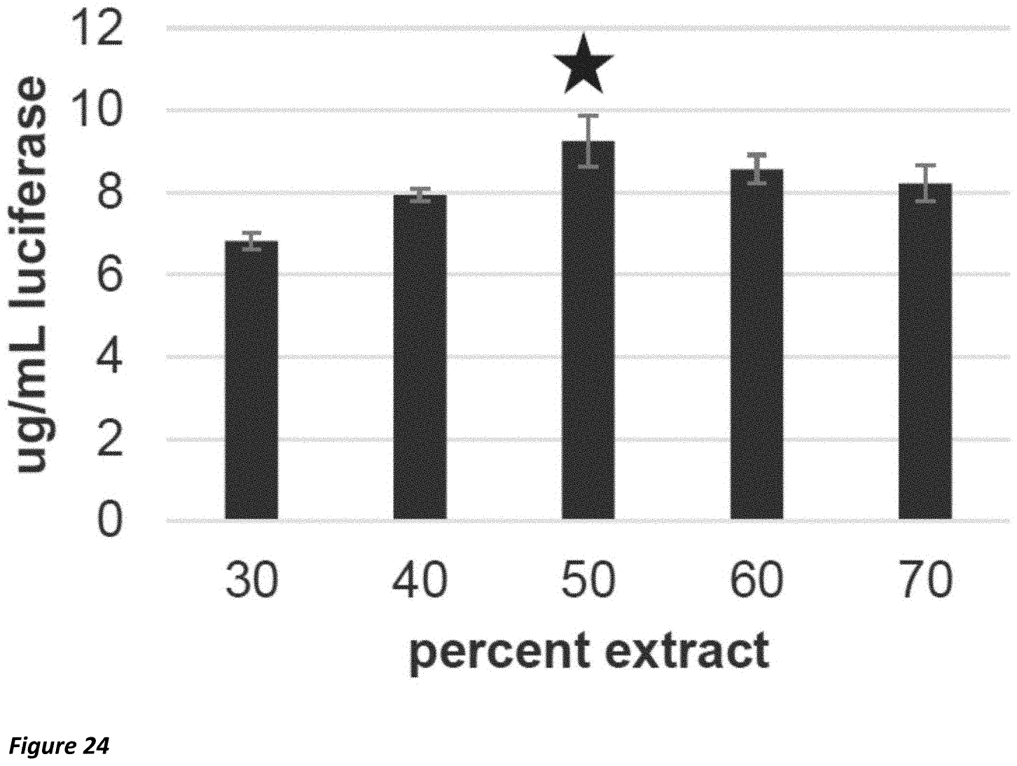

We also assessed how the concentration of chloroplast extract could influence protein production in our chloroplast extract CFPS system. FIG. 24 illustrates the effect of chloroplast extract concentration on protein production in a cell-free system comprising the chloroplast extract. Protein production was optimal at an extract concentration of between about 40-70% (e.g., about 50-60%).

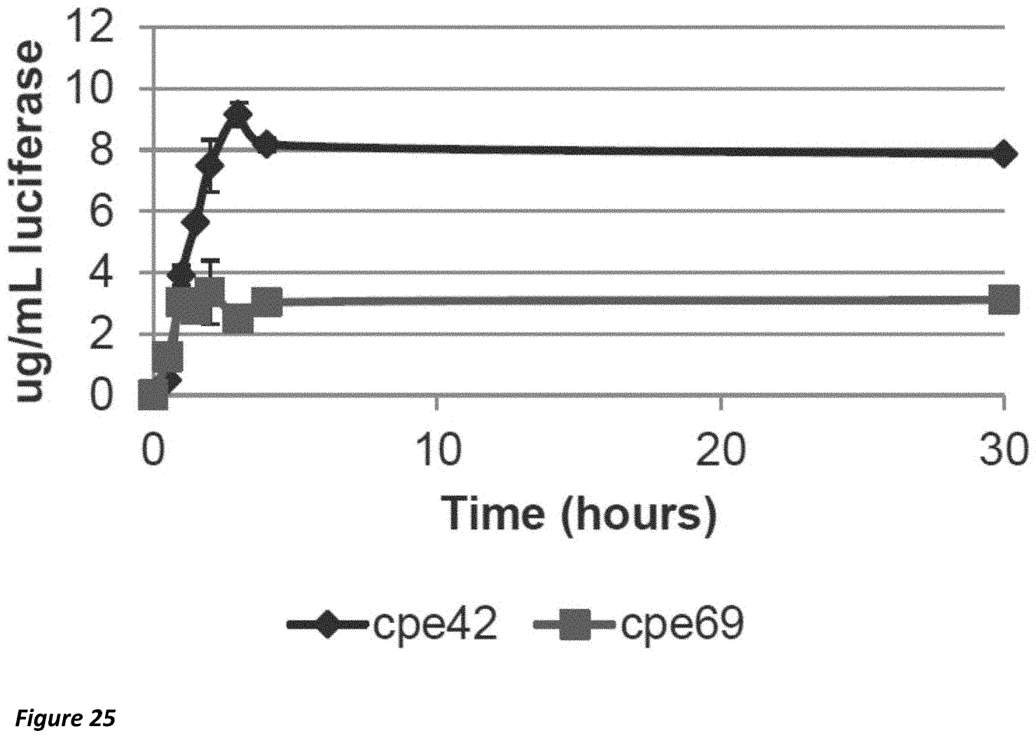

We also assessed protein production versus time. FIG. 25 illustrates protein production versus time in two different cell-free systems comprising a chloroplast extract. We observed that protein production increased from time 0-3 hours and achieved a steady state afterward.

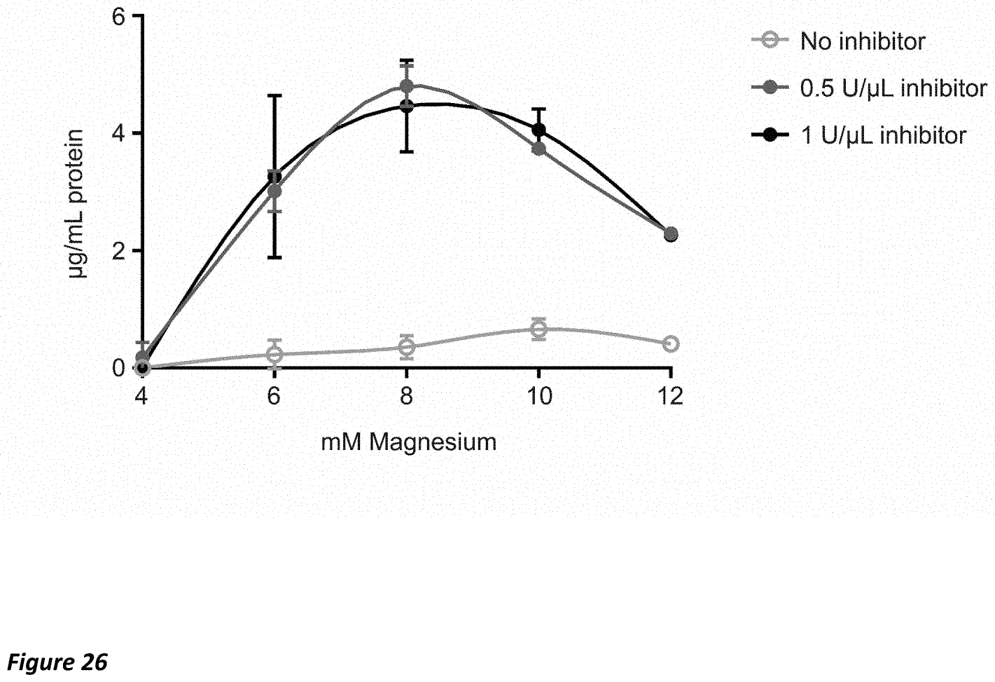

We also assessed whether we could improve protein production by inhibiting mRNA degradation. FIG. 26 shows the effect of RNAse inhibitor on chloroplast cell-free reaction. 0, 0.5 and 1 U/μL RNAse inhibitor was added. The results demonstrate that 0.5 U/μL RNAse inhibitor enhances protein production of the cell-free system, but that 1 U/μL RNAse inhibitor has no additional effect.

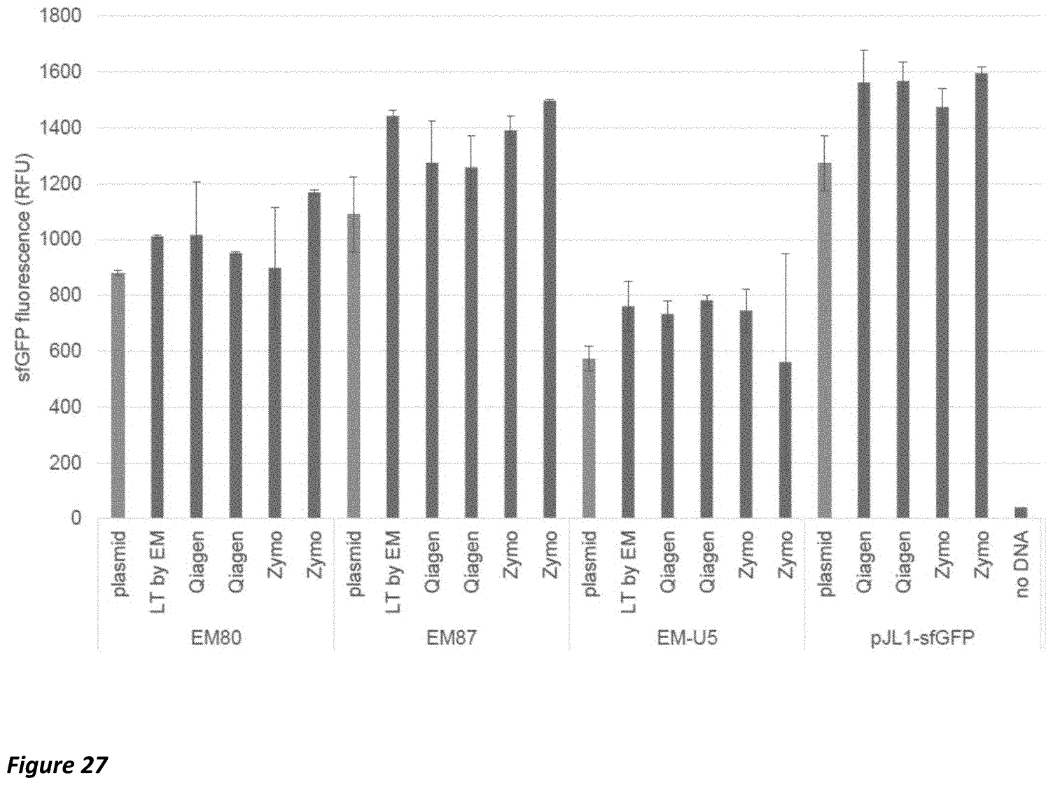

We also assessed whether we could utilize linear DNA templates for protein production. FIG. 27 illustrates that linear templates or plasmid DNA can be used in chloroplast cell free reactions to produce protein, with linear templates offering a slight boost to yields and a faster turnaround time than plasmids. Linear templates prepared by PCR, then purified with a Qiagen cleanup kit (LT by EM) work as well as plasmid DNA. Also, DNA prepared with a Qiagen or ZymoPCR cleanup kit work equally well as a plasmid DNA as a DNA template for protein production.

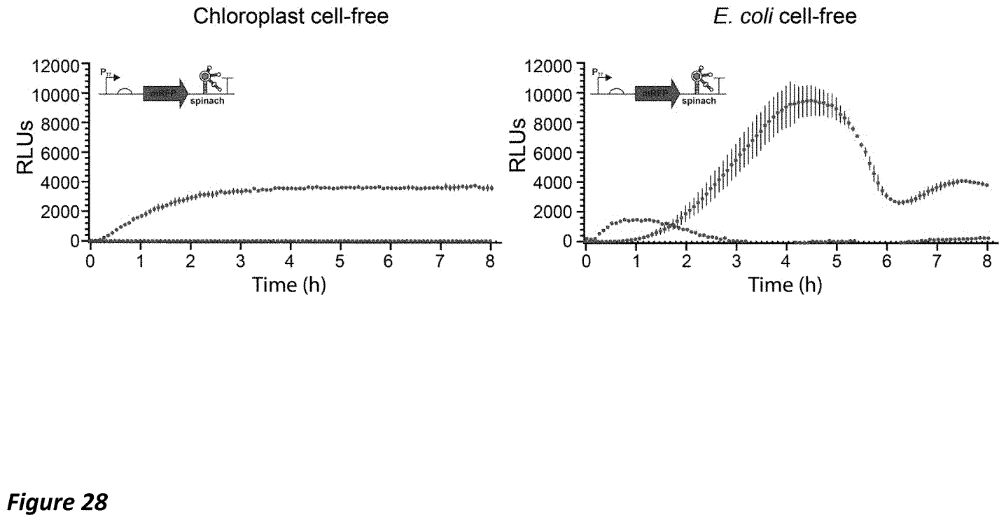

Finally, we compared mRNA expression dynamics in our chloroplast extract CFPS system versus an E. coli extract CFPS system. FIG. 28 illustrates different mRNA expression dynamics in chloroplast and E. coli cell-free systems. A construct (pY71mRFP1SpA) expressing a fluorescent protein (mRFP) and a fluorescent RNA aptamer (spinach) was assayed in both chloroplast and E. coli cell-free systems. The results in FIG. 28 show that mRNA levels are stable in the chloroplast cell-free system during 8 hours (or more).

### Example 2—Methods of In Vitro Protein Synthesis and Prototyping of Genetic Expression in Plants Utilizing Cell-Free Protein Synthesis Platforms Comprising Chloroplast Extracts

Abstract

Chloroplasts are attractive targets of plant engineering with high foreign protein production capacity and genetic control that enables the expression of several genes stacked into operons. Transferring multi-genic functions requires the characterization of genetic parts and fine-tuning of protein expression, but the time-consuming transformation of chloroplasts prevents engineers from carrying out this task in plants. To overcome this problem, a cell-free system—based on purified and subsequently lysed tobacco chloroplasts—was used which enables the expression of proteins from a template. This can be used to assess the impact of ribosome binding sites, terminators, promoters, and other genetic parts, as well as codon optimization on gene expression prior to testing in a host plant. The chloroplast cell-free system prevents mistakes like using wrong codon-optimization or ribosome binding sites, and could allow for 10,000's or more genetic constructs to be prototyped weekly. Those producing the desired expression pattern could be selected for plastid transformation in the plant. In addition, the cell-free system could also serve as a test-bed to finely balance expression of multi-gene clusters.

Applications

Applications for the disclosed technology include, but are not limited to: (i) In vitro screening of enzyme pathway variants for plant metabolic engineering; (ii) Pre-screening DNA designs with chloroplast CFPS allows for a dramatic reduction in iteration time for research and development; (iii) Synthetic biology applications; (iv) Fundamental studies of the chloroplast translation apparatus and harnessing translation machinery for novel functions; (v) High-throughput interrogation and characterization of organismal genetic parts (promoters, terminators, ribosome binding sites, etc.); and (vi) Agriculture and Crop Science.

Advantages

The current state of the art in chloroplast transformation takes approximately one year to produce transformants, meaning that design iterations occur annually. Chloroplast transformation is a laborious process that involves bombardment of plant tissue with gold particles loaded with DNA. This method is not sufficiently high-throughput for prototyping genetic parts. The disclosed technology can be utilized to significantly curtail the amount of time needed to perform multiple genetic tests. For example, instead of performing 10 genetic tests in 9 month, one could perform 10,000 tests in a week.

While it is possible to use plant cell culture to study candidates for transformation into plants and some plant cell lines such as BY-2 cell lines contain chloroplasts, their chloroplasts are undifferentiated, and therefore less suitable to mimic cellular processes of a fully developed functional chloroplast. Similarly, wheat germ extract could be used to prototype nuclear modifications. However, the chloroplast is a more desirable location to stably express exogenous DNA because there is no silencing of organelle DNA. In addition, several genes can be expressed from an operon and the maternal inheritance of this subcellular compartment prevents exogenous gene escape through pollen.

Brief Summary of Technology

Cell-free protein synthesis (CFPS) is fueling numerous applications as a powerful in vitro expression system. Yet, there is little available for in vitro genetic prototyping for plant biologists. We have applied our expertise in cell-free systems to improve and show utility of a CFPS from chloroplasts. Currently, we have demonstrated a powerful approach for prototyping genetic parts in cell-free systems. The technology is further described in FIGS. 1-5. We expect that this technology will be highly desirable to large biotechnology companies that seek to prototype on a more rapid timescale.

Problems Solved

Problems solved by the disclosed technology include but are not limited to the following aspects. Technological developments in plant biology have historically been slow, in spite of the fact that our early understanding of genetics was pioneered in plants. Previously, prototyping in BY-2 cell culture has informed transformation into the nuclear genome, though this is not necessarily a high-throughput process and nuclear transformation is susceptible to gene silencing. Transformation into the chloroplast is more generationally stable, but is highly time-consuming, requiring one year to produce transformants. There is also no known way of pre-screening candidates for transformation in a chloroplast-like environment. As a result, the current compromise is to devote a staggering number of person-hours to development of a small number of candidates or to prototype in a nuclear context.

Commercial Aspects

Commercial aspects of the disclosed technology include but are not limited to the following. Chloroplasts are attractive targets of plant engineering with high foreign protein production capacity and genetic control that enables the expression of several genes stacked into operons. Transferring multi-genic functions requires the characterization of genetic parts and fine-tuning of protein expression, but the time-consuming transformation of chloroplasts prevents engineers from carrying out this task in plants. To overcome this problem, a cell-free system—based on purified and subsequently lysed tobacco chloroplasts—was used which enables the expression of proteins from a template. This technology could be combined with the well-understood practice of tobacco chloroplast transformation to produce large amounts of medically or agriculturally relevant compounds. Alternatively, our methods could be applied to chloroplasts derived from therapeutically or agriculturally relevant species to better prototype in more distantly related plant species such as grasses, trees, or algae. Our initial discovery has opened up the possibility to manipulate levels of natural products in many different areas of plant biotechnology.

This system is the first ever chloroplast-derived CFPS system capable of transcription and translation and is more high-yielding than previously reported translation-only systems. While other plant-derived systems have been published (wheat germ extract and BY-2 extract), these systems are not suited to prototyping for the chloroplast, which is a more stable transformation cite than the nucleus. This system will enable technological discovery by generating a method for rapid prototyping in a plant context.

Results

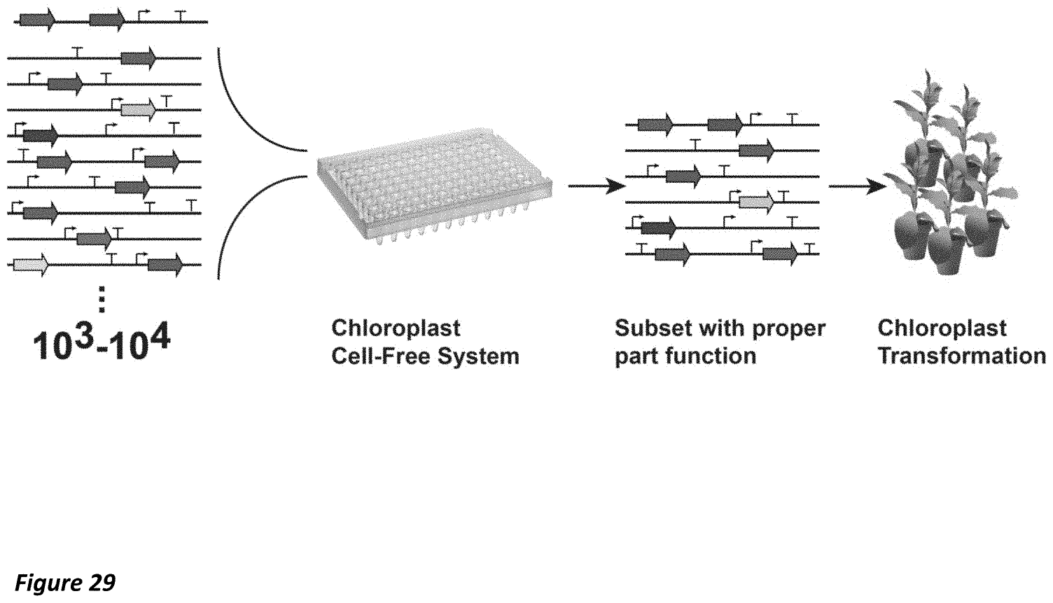

We set out to design a system for rapid plastid prototyping using a chloroplast extract CFPS system. FIG. 29 provides a schematic illustration of plastid prototyping using a chloroplast cell-free system and subsequent chloroplast transformation to prepare modified plants. As illustrated, a library of modified genetic components (e.g., ribosome binding sites (RBS)) comprising >10³ or 10⁴ members can be screened using a chloroplast CFPS system in order to identify members with desirable characteristics (e.g., optimal protein production). After the components are identified, the components can be utilized to prepare expression vectors for chloroplast transformation.

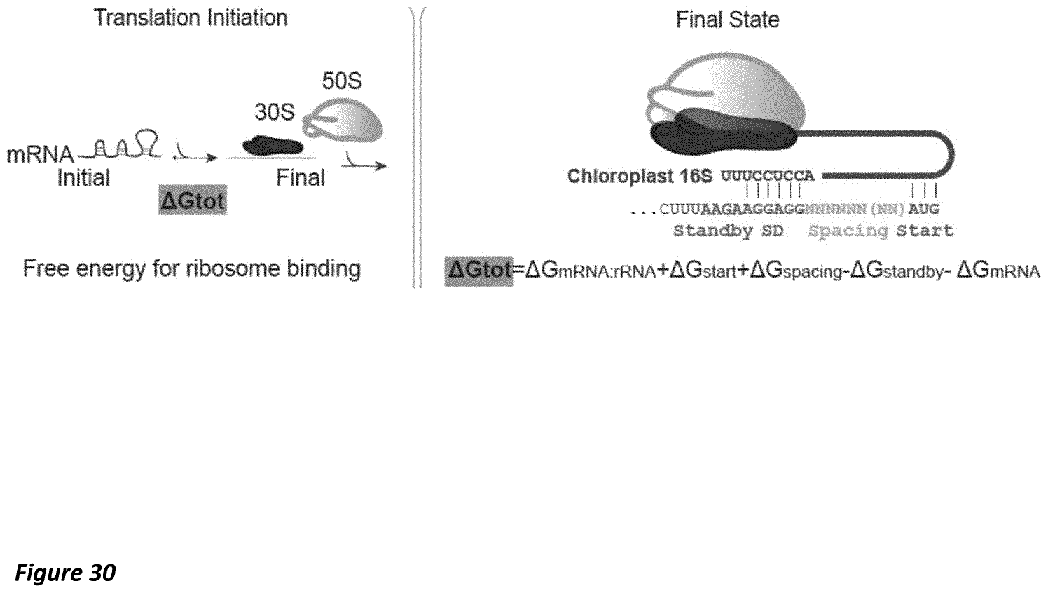

We set out to devise a model for designing and testing ribosome binding sites (RBS) based on binding strengths. FIG. 30 illustrates a thermodynamic model that provides the basis for designing ribosome binding sites (RBS) that have different strengths. The thermodynamic model is based on the interaction between an mRNA transcript and the 30S ribosomal complex. This thermodynamic model is part of our so-called “RBS calculator.” Our RBS calculator can be used as a computation tool that can design RBS's with different binding strengths. The amount of protein expressed in CFPS systems typically correlates with RBS strength. Our RBS calculator can be used for chloroplasts because many plastid 5′-UTR's have Shine Delgamo-like RBS's.

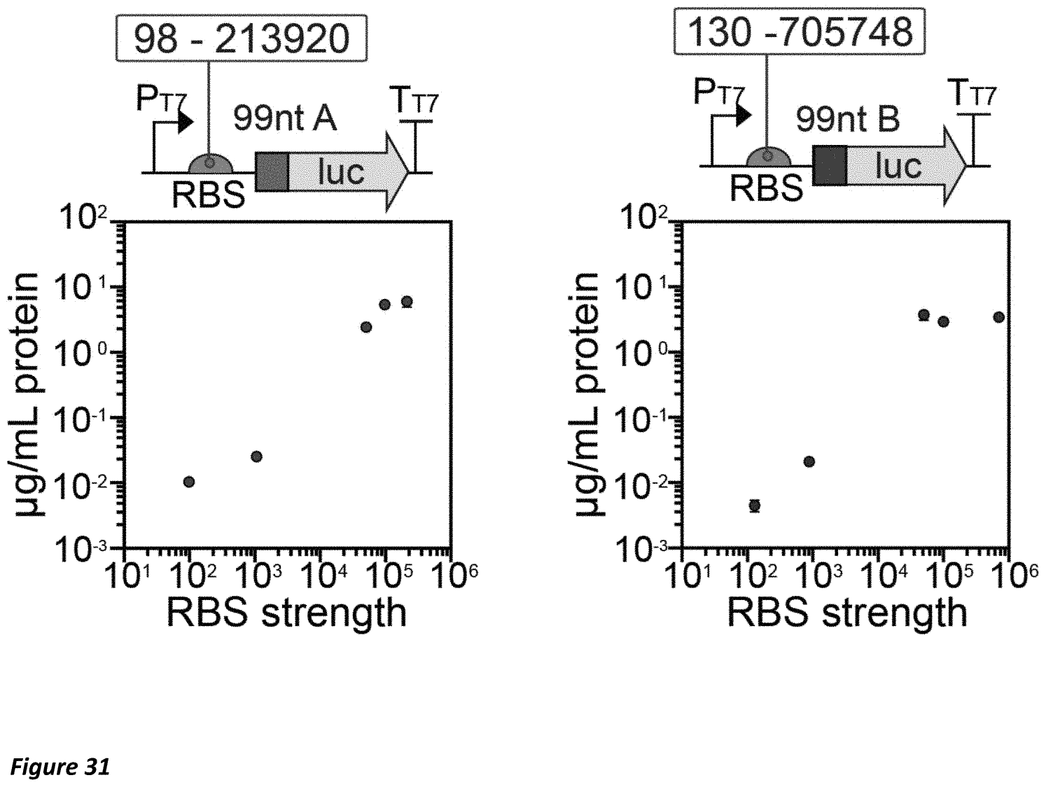

We created an expression library of 5′-UTR's operably linked to an expressed protein comprising the first 99 nucleotides of one of two biosynthetic enzymes A (Kas) and B (AroG) (from a plant biosynthetic pathway for capsaicin biosynthesis) fused to luciferase. FIG. 31 illustrates the results of experiments in which protein expression was measured versus ribosome binding strength for the library of 5′-UTR's. A strong correlation between RBS strength and measured protein output was observed.

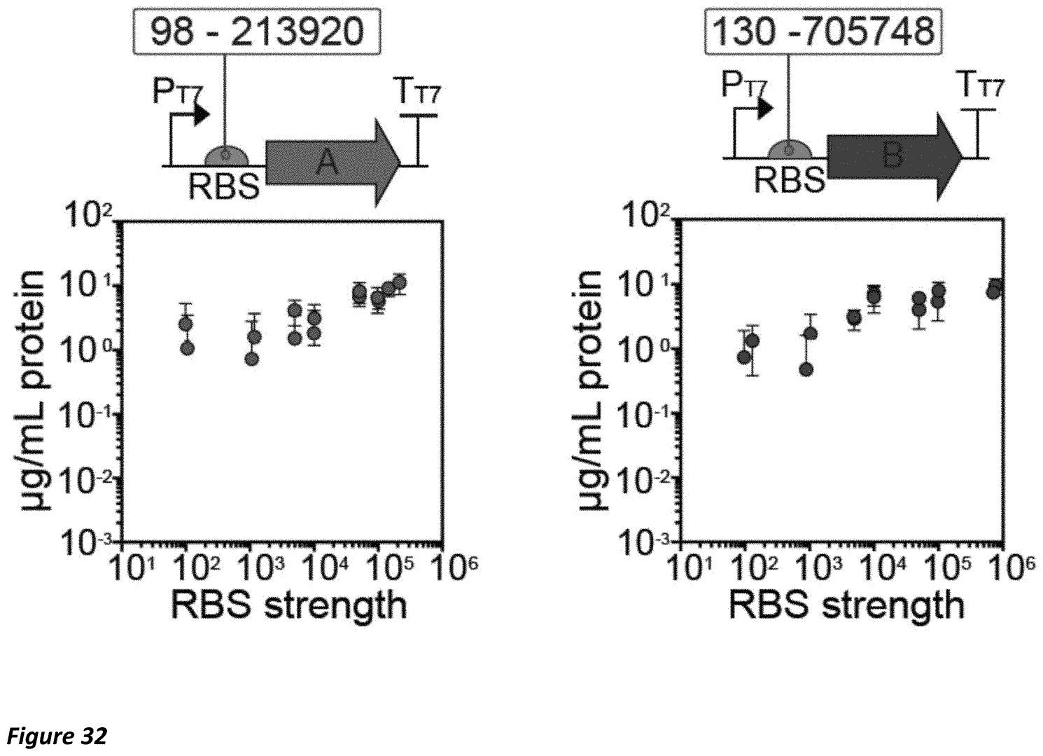

We also assessed protein expression of the full-length enzymes A (Kas) and B (AroG) in our chloroplast extract CFPS system. FIG. 32 illustrates the results of experiments in which protein expression was measured versus ribosome binding strength for a library of expression vectors having different ribosome binding sites (RBS) and expressing one of biosynthetic enzymes A or B. Protein production was measure via incorporation of radioactive leucine. Twenty eight (28) variants with different RBS binding strengths were tested in less than four (4) weeks.

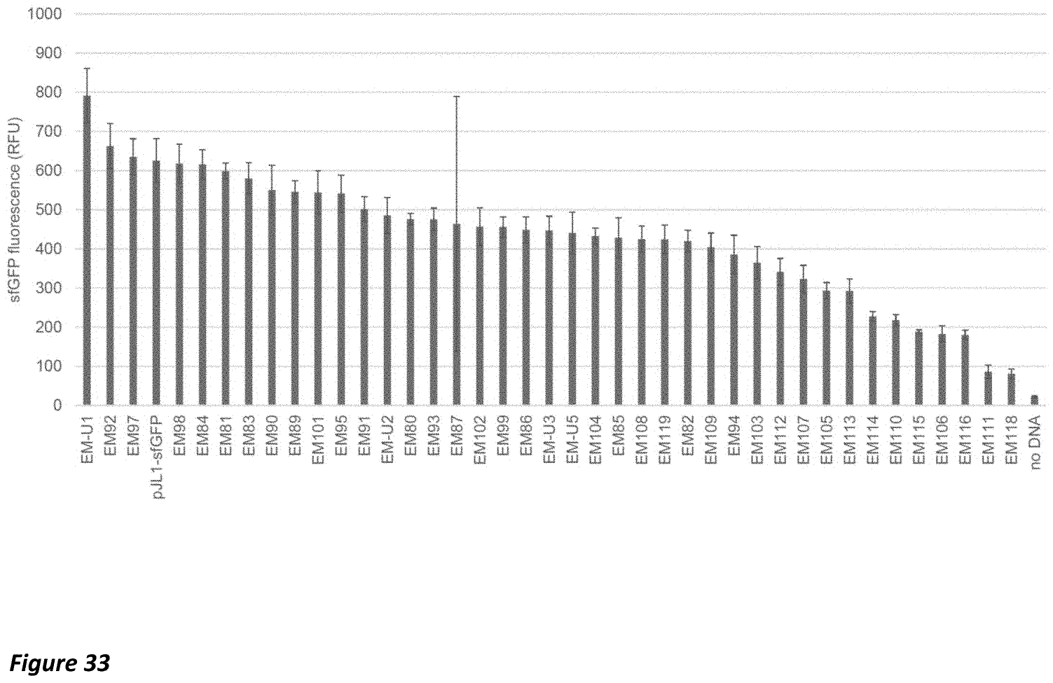

Our methods for assessing genetic components such as RBS also could be automated to increase throughput. FIG. 33 illustrates how a chloroplast extract cell-free system can be adapted as part of an automated system. Yields of protein (GFP) in a chloroplast extract using an RBS library and a 16S rRNA sequence from the chloroplast illustrate how a chloroplast cell-free system can be utilized to rank the efficiency of genetic parts such as RBS in an automated system.

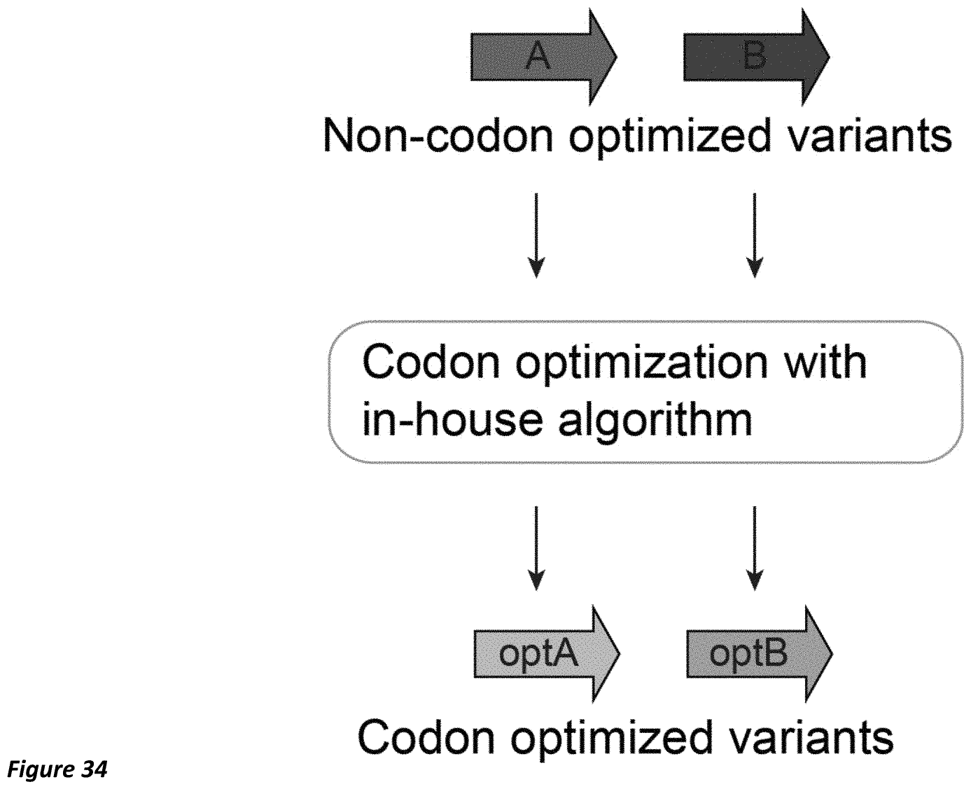

We also assessed whether we could introduce further modifications in our expression template to improve protein production such as codon optimization. FIG. 34 illustrates schematically how the effect of codon optimization on protein production can be assessed using a chloroplast cell-free system and how an algorithm can be devised from results in a chloroplast cell-free system to provide an algorithm for codon optimization. Our chloroplast extract CFPS system can then be utilized to assess protein production based on codon optimization prior to transformation of a chloroplast with an expression vector.

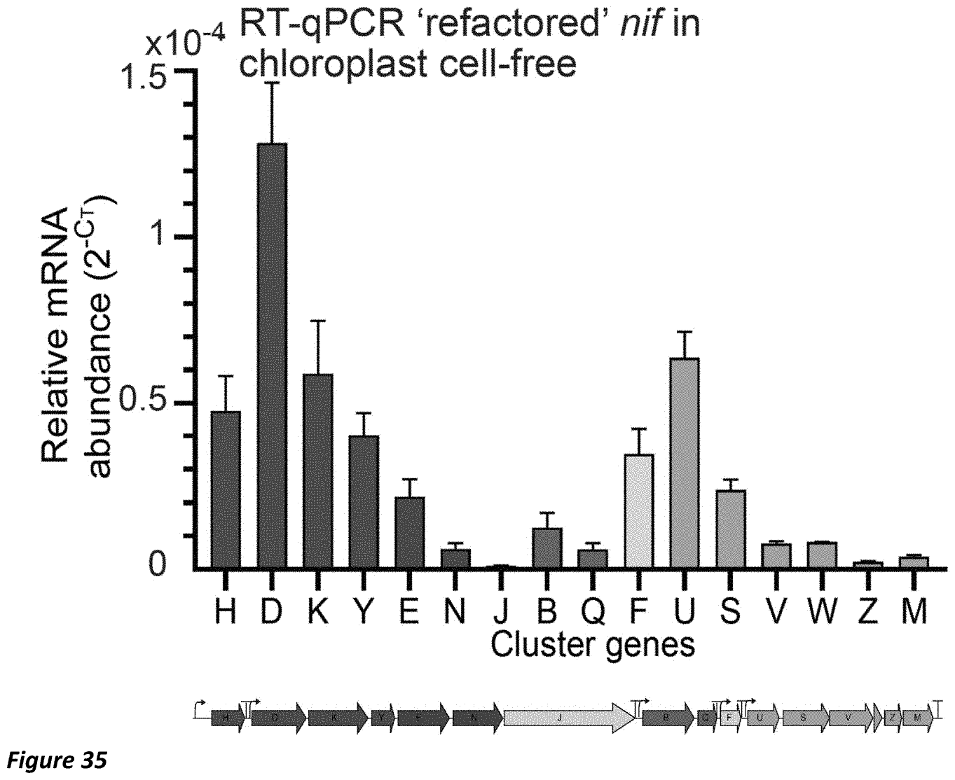

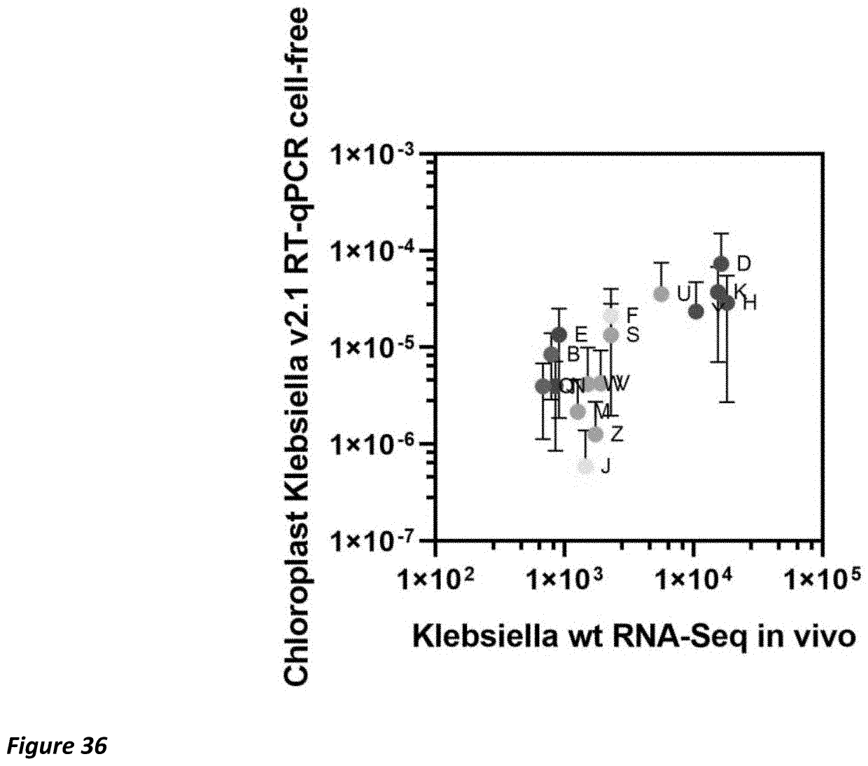

We also assessed how we might utilize our chloroplast extract CFPS system to assess and tune expression of multi-gene clusters. FIG. 35 illustrates RT-qPCR analysis of a multi-genic cluster in a chloroplast cell-free system. A cluster (Klebsiella‘refactored’ v2.) composed of (sixteen) 16 genes (20 kb) was used as template for chloroplast cell-free reactions. Reactions were incubated 25° C. during 1 h and 100 μL of the reaction mix was harvested for total RNA extraction. Total RNA was assayed with RT-qPCR. All 16 genes were detected in both cell-free systems, demonstrating that the chloroplast cell-free is able to express multi-gene cluster. We then compared expression in vivo to our results obtained in our CFPS system. FIG. 36 provides a comparison of multi-gene expression per FIG. 36 versus multi-gene expression in vivo which demonstrates a strong correlation, indicating that our CFPS system can be utilized to predict and tune expression after chloroplast transformation.

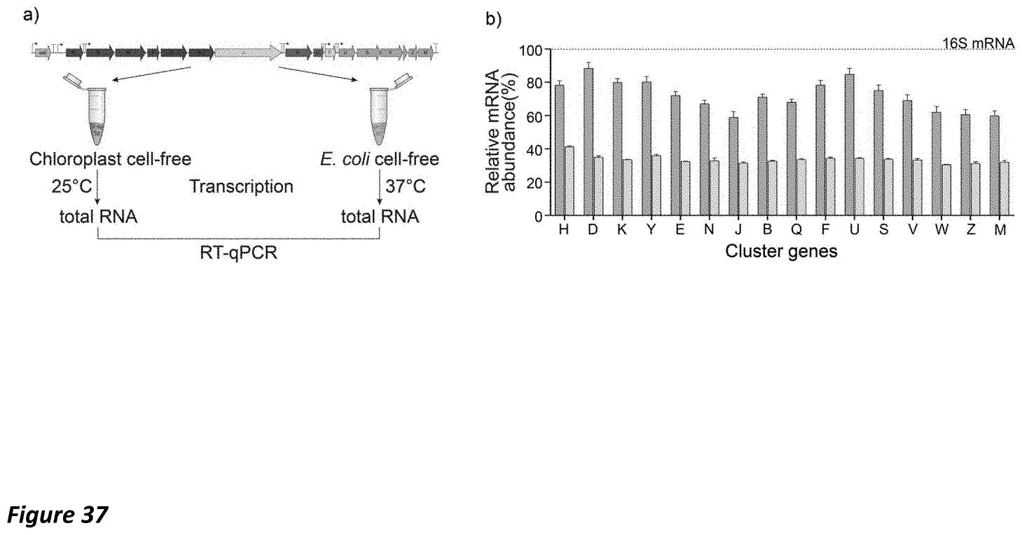

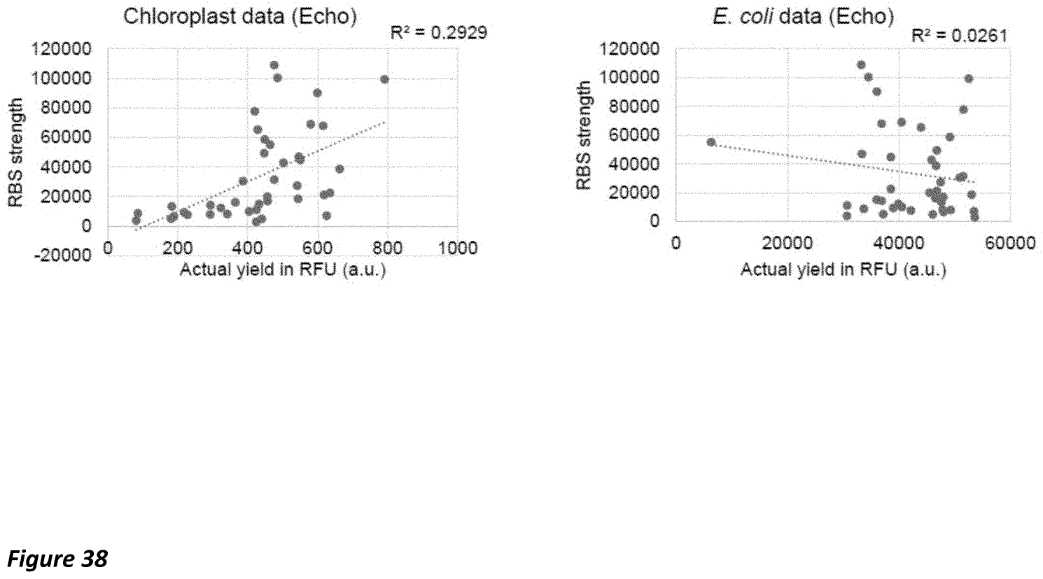

We compared expression of our sixteen (16) multi-gene cluster in a chloroplast extract CFPS system versus an E. coli extract CFPS system. FIG. 37 illustrates RT-qPCR analysis of a multi-genic cluster in chloroplast and E. coli cell-free systems. All 16 genes were detected in both cell-free systems, demonstrating that the chloroplast cell-free is able to express a multi-gene cluster. However, the observed correlation between RBS binding and protein production was stronger in our chloroplast extract CFPS system than in a CFPS that utilized an E. coli extract. (See FIG. 38).

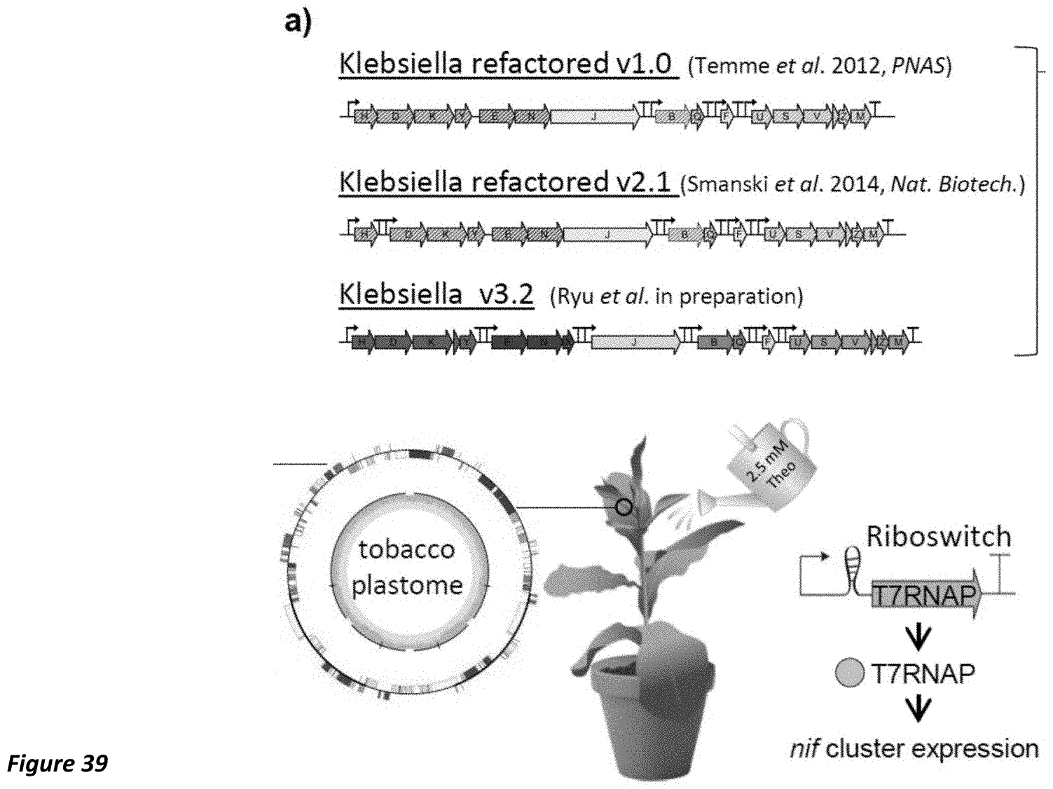

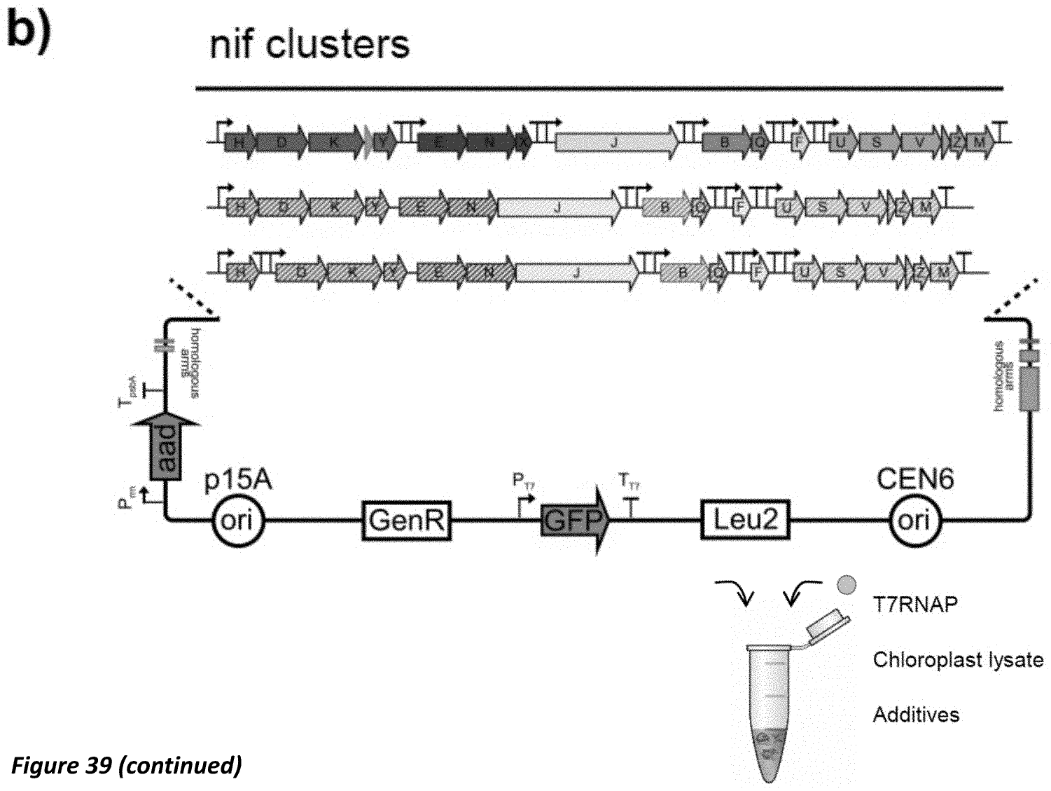

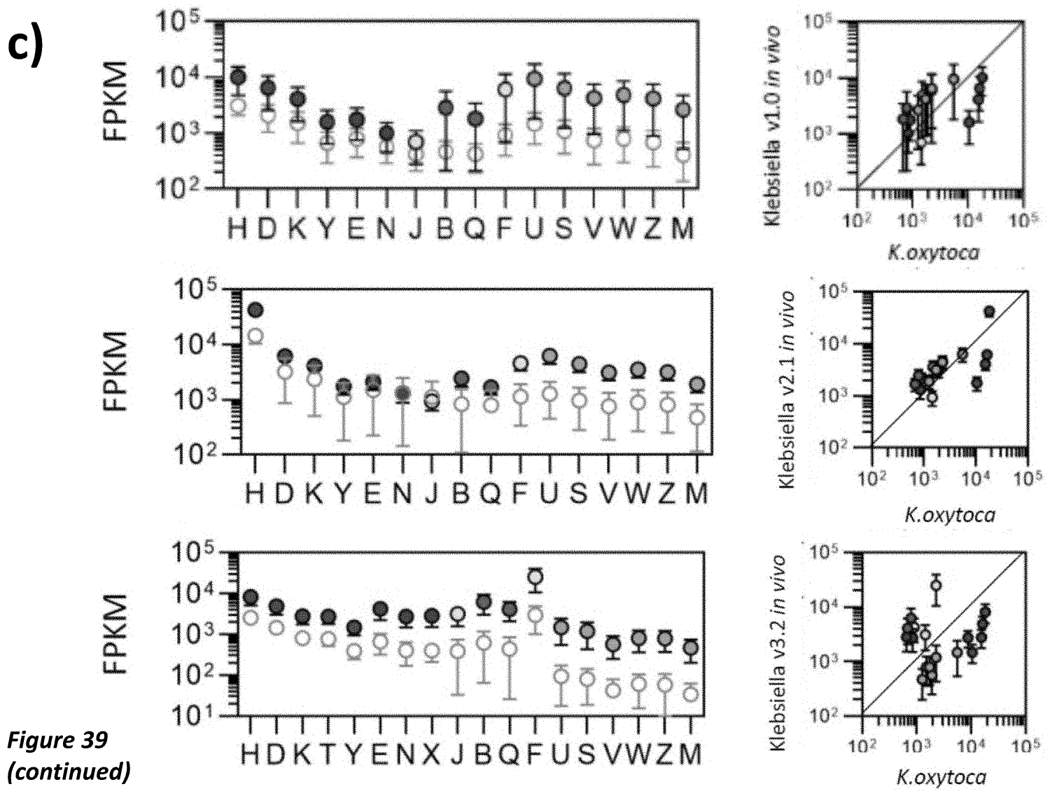

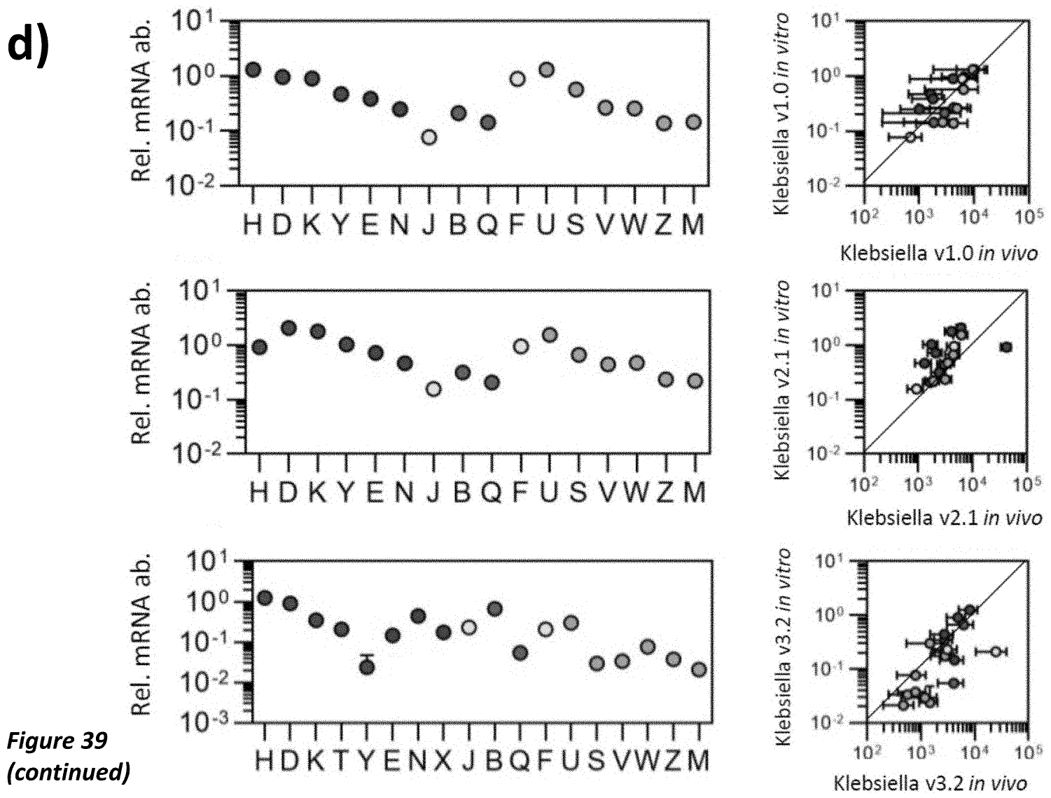

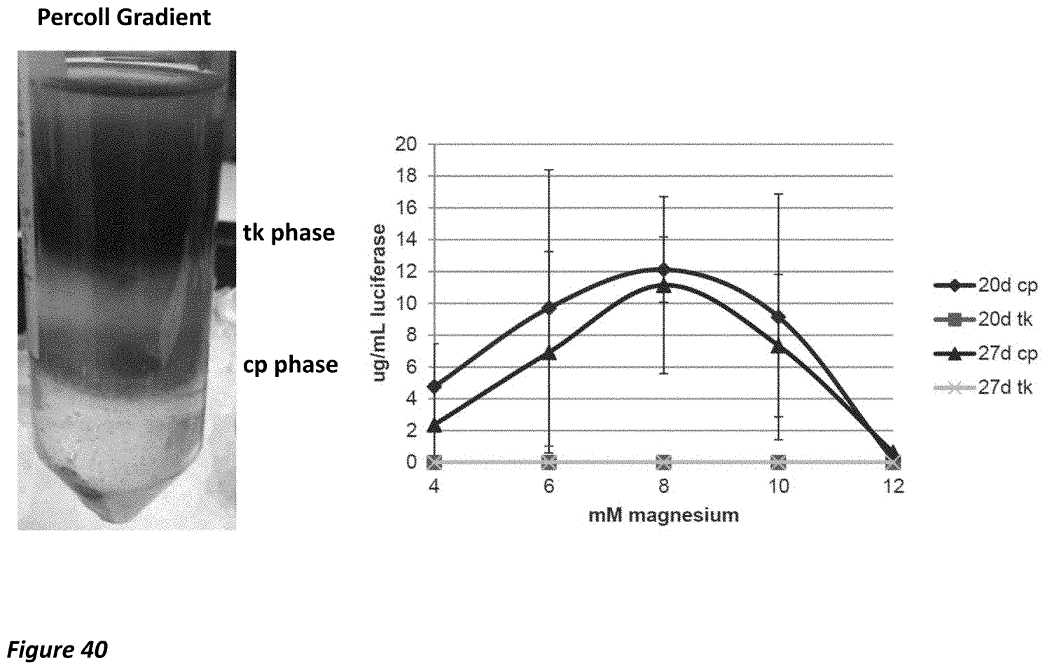

We also assessed whether chloroplast in vivo gene expression would correlate with chloroplast cell-free data. (See FIG. 39). First, we integrated three (3) engineered nitrogenase (nif) clusters (Klebsiella v1.0, v2.0, and v3.2) into the tobacco plastome under expression from T7 promoters. (See FIG. 39 a) and b)). Upon theophylline (Theo) induction, plastome integrated T7 RNAP transcribed the nif clusters. We observed a good correlation between expression in vivo and expression in our chloroplast extract CFPS system.

### Example 3—Preparation of Maize Chloroplast Extracts and Use Thereof in Cell-Free Protein Synthesis

FIG. 20 illustrations the preparation of chloroplast extracts from maize and the use thereof for cell-free protein synthesis. Leaves were collected after roughly 6 hours of light exposure, cut from plants at 20 or 27 days post-planting, and cut into small pieces. Approximately 200 g leaves were collected and transferred to 4° C. These were blended in ˜600 mL MCB1 buffer with polyvinylpyrrolidone (the same buffer as used with tobacco) for 5 second bursts for a total of 20 seconds, then strained exactly as is done for tobacco chloroplasts. This liquid was spun at 1000×g for 4 minutes, the pellet was collected, and resuspended in 9 mL MCB1 buffer, then loaded onto stepwise Percoll gradients with no more than 4.5 mL per gradient. All buffers and gradients are the same as were used with tobacco chloroplasts.

Gradients were centrifuged at 10000×g for 10 minutes with minimum acceleration and brake and the lower band was collected as chloroplasts (cp). The upper band was collected as loose thylakoids (tk) or broken chloroplasts to check for activity in this phase. These populations were washed three times in MCB2 as described for tobacco, resuspended in the same lysis buffer used for tobacco chloroplasts at 1 mL/g, and flash frozen and stored at −80.

Extracts were prepared from the resuspended chloroplasts after thawing. Resuspended chloroplasts were passed through a 25G needle 12 times and then centrifuged at 30000×g for 30 minutes twice as described for tobacco, but no dialysis was performed. Extracts were flash frozen and stored at −80.

### Example 4—Maize Plastid Cell-Free System and Uses Thereof for Cell-Free Protein Synthesis and Prototyping Gene Expression

Background Almost half of the world total grain production comes from maize. One third of this is genetically modified (GM) maize. The foreign genes in these GM maize varieties are inserted into the nuclear genome which could transfer them into non-GM varieties or related species through open pollination, resulting in the appearance of foreign genes in non-GM kernels. Expressing these genes in the plastid genome of maize would considerably reduce the risk because plastids are maternally inherited and do not spread with the pollen. The maize plastid transformation technology has not been utilized effectively because of time-consuming transformation and the lack of part libraries that drive expression of foreign genes at appropriate levels. Here, we disclose a maize plastid cell-free system that may be utilized for prototyping gene expression.

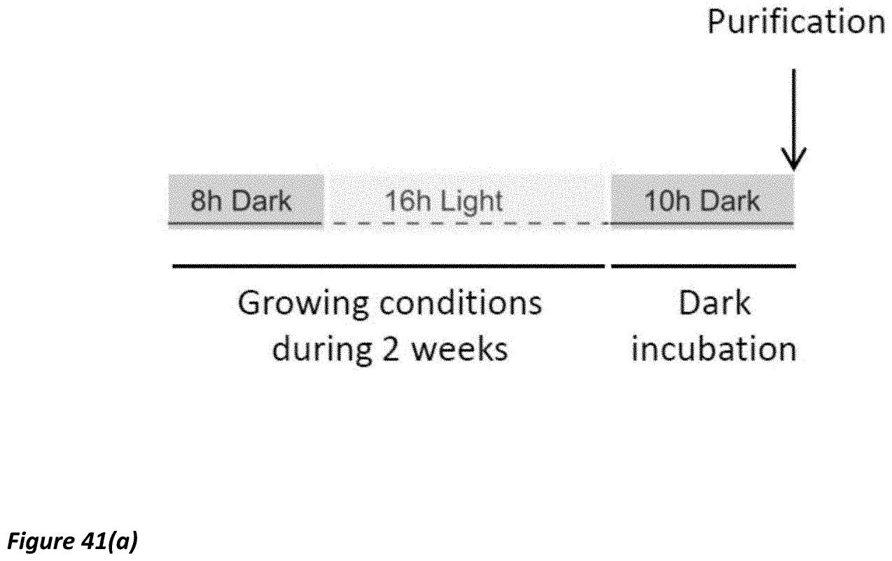

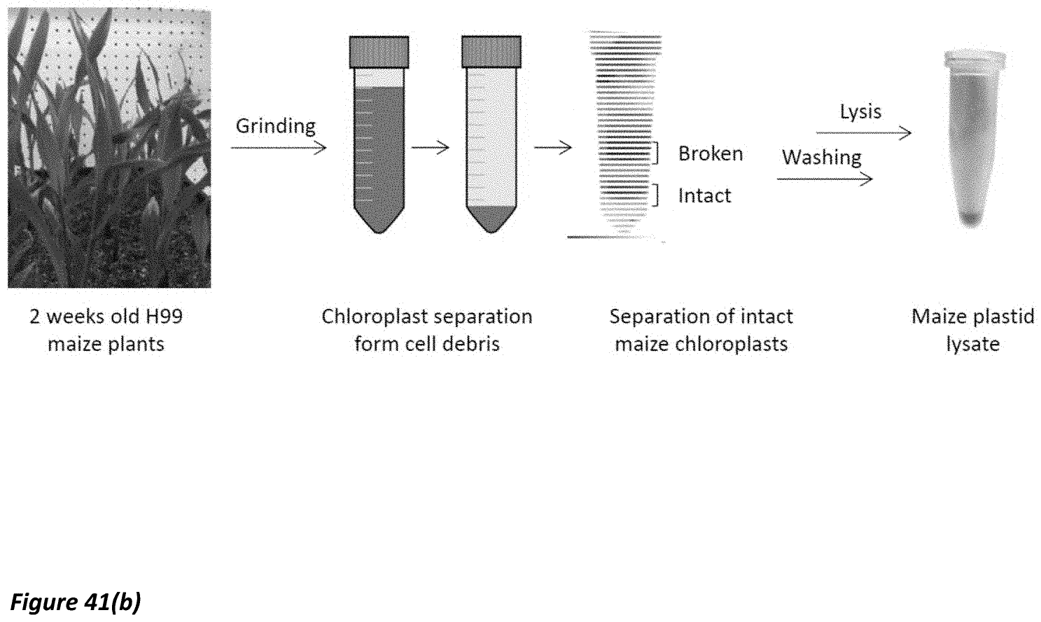

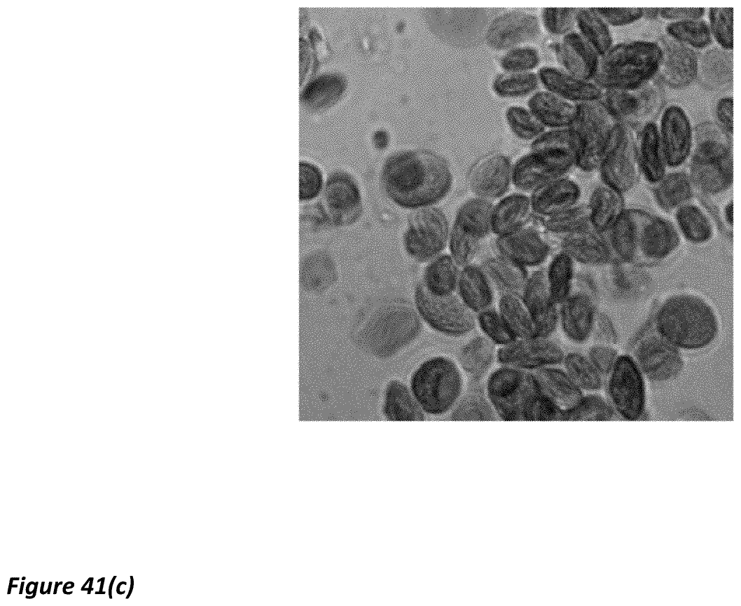

FIG. 41(a), FIG. 41(b), and FIG. 41(c) shows the process of preparing and purifying the maize cell-free system. Growing conditions of maize plants used to prepare the system are shown in FIG. 1(a). Zea mays subsp. mays cv. H99 was cultivated during 2 weeks on 8 h light and 16 h dark light conditions. These plants were incubated in the dark for 10 h before purification. Purification of maize plastid cell-free system is shown in FIG. 41(b). After breaking the cells, chloroplasts (green plastids) from leaves were separated through several rounds of centrifugation which includes a separation of broken and intact chloroplasts in a Percoll density gradient. A light microscope image of purified intact chloroplasts with no visible starch granules is shown in FIG. 41(c) (result of the dark incubation shown in FIG. 41(a). The absence of starch granules protects chloroplasts during the purification and results in higher activity of the purified lysate.

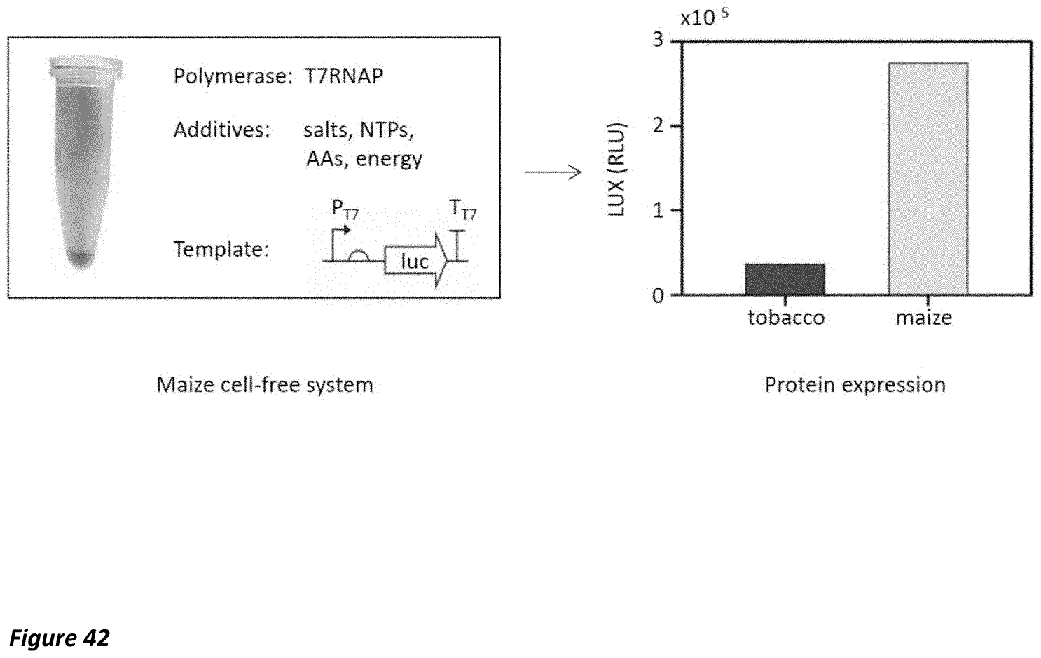

Maize cell-free systems were demonstrated to synthesize protein from a DNA template. FIG. 42 shows a schematic of the reaction—a DNA template expressing the luciferase gene under the control of the T7 RNA Polymerase was assayed in the maize plastid cell-free. As shown in the graph of FIG. 42 the system was able to transcribe and translate luciferase protein. Luminescence (LUX) was detected.

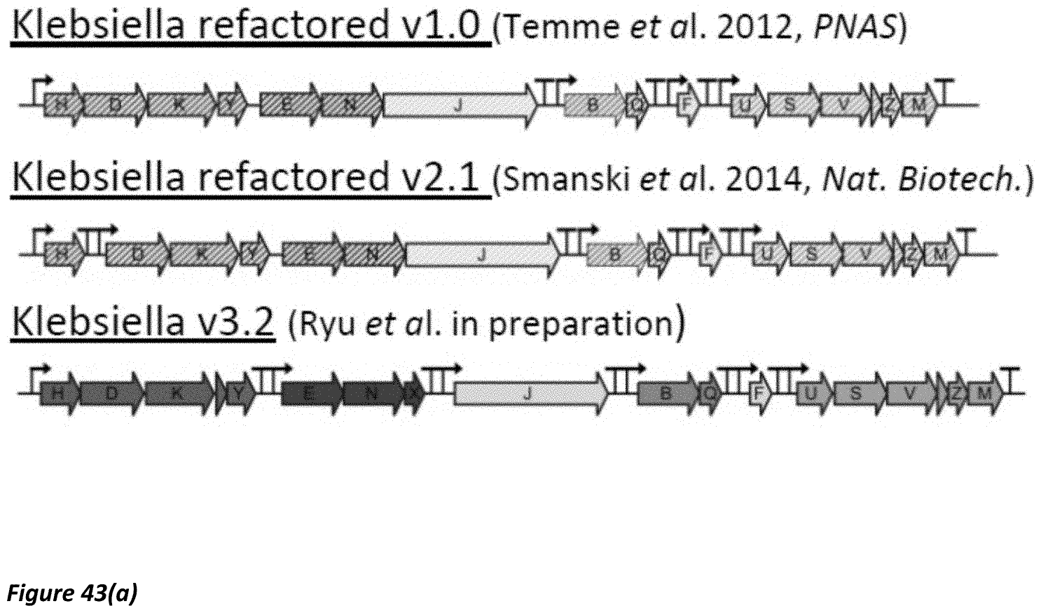

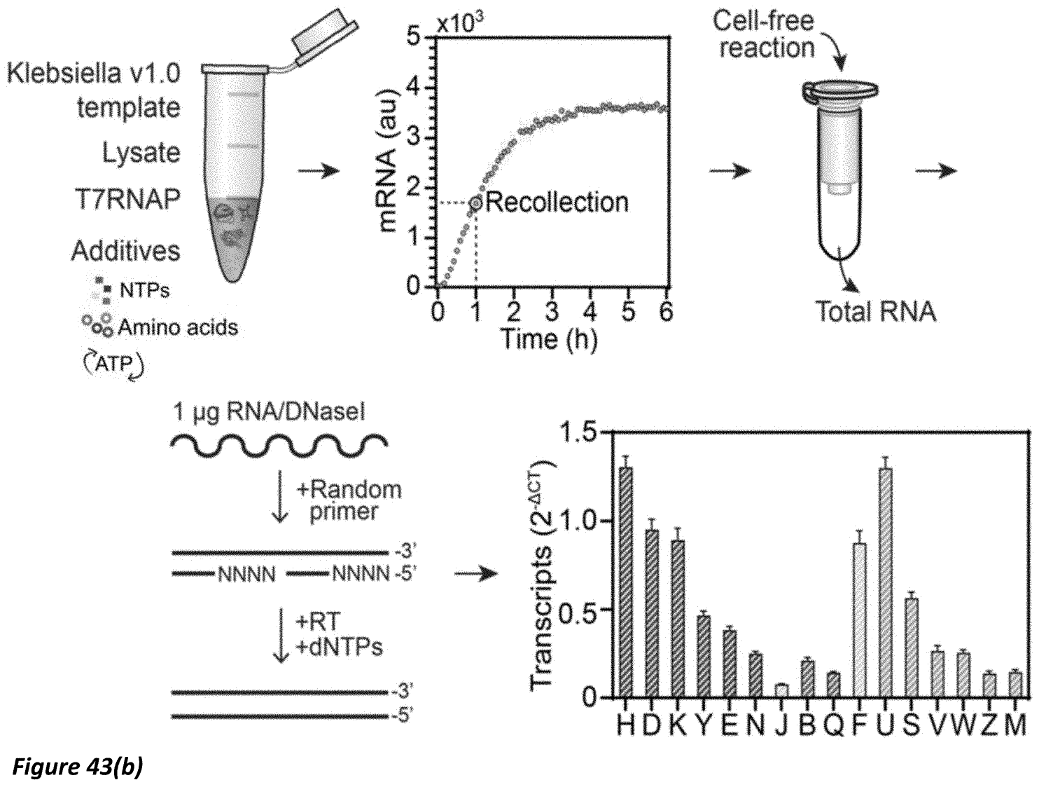

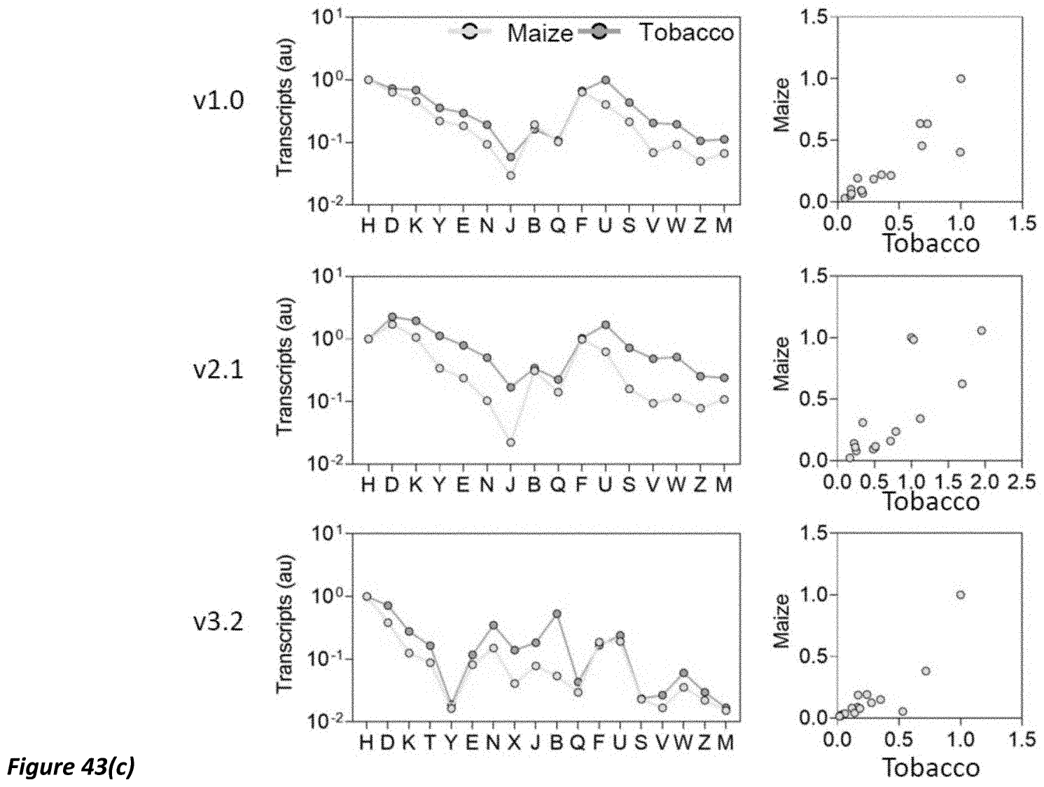

The maize cell-free system described above was analyzed to determine feasibility in producing proteins from a multi-genic construct (gene cluster). Genetic clusters encoding nitrogenase genes (Nif) were incubated in the cell free system and expression levels were analyzed. The data is shown in FIG. 43(a), FIG. 43(b), and FIG. 43(c). The data generated in the cell-free system was able to predict mRNA expression levels of several multigenic constructs coding for nitrogenase. FIG. 43(a) is a schematic depicting the templates coding for nitrogenase and the reaction components of the maize plastid cell-free system. The maize cell-free system was able to produce mRNA and protein from the DNA template. FIG. 43(b) shows the steps of purification of total RNA from the maize cell-free reaction and cDNA library synthesis for RT-qPCR. FIG. 43(c) shows the data on performance of maize cell-free system at the mRNA level expressing nitrogenase clusters. Comparison with a tobacco cell-free system control showed distinct differences in cluster transcription, presumably due to species specific expression.

### REFERENCES

### Non-Patent References

1. Carlson, E. D., et al., Cell-free protein synthesis: applications come of age. Biotechnol Adv, 2012. 30(5): p. 1185-94.
2. Martin, R. W., et al., Development of a CHO-Based Cell-Free Platform for Synthesis of Active Monoclonal Antibodies. ACS Synth Biol, 2017. 6(7): p. 1370-1379.
3. Martin, R. W., et al., Cell-free protein synthesis from genomically recoded bacteria enables multisite incorporation of noncanonical amino acids. Nat Commun, 2018. 9(1): p. 1203.
4. Lu, Y., Cell-free synthetic biology: Engineering in an open world. Synth Syst Biotechnol, 2017. 2(1): p. 23-27.
5. Jewett, M. C. and J. R. Swartz, Mimicking the Escherichia coli cytoplasmic environment activates long-lived and efficient cell-free protein synthesis. Biotechnol Bioeng, 2004. 86(1): p. 19-26.
6. Caschera, F. and V. Noireaux, Synthesis of 2.3 mg/ml of protein with an all Escherichia coli cell-free transcription-translation system. Biochimie, 2014. 99: p. 162-8.
7. Zawada, J. F., et al., Microscale to manufacturing scale-up of cell-free cytokine production—a new approach for shortening protein production development timelines. Biotechnol Bioeng, 2011. 108(7): p. 1570-8.
8. Jewett, M. C., et al., An integrated cell-free metabolic platform for protein production and synthetic biology. Mol Syst Biol, 2008. 4: p. 220.
9. Buntru, M., et al., Tobacco BY-2 cell-free lysate: an alternative and highly-productive plant-based in vitro translation system. BMC Biotechnol, 2014. 14: p. 37.
10. Buntru, M., et al., A versatile coupled cell-free transcription-translation system based on tobacco BY-2 cell lysates. Biotechnol Bioeng, 2015. 112(5): p. 867-78.
11. Thoring, L., et al., High-yield production of “difficult-to-express” proteins in a continuous exchange cell-free system based on CHO cell lysates. Sci Rep, 2017. 7(1): p. 11710.
12. Penalber-Johnstone, C., et al., Optimizing cell-free protein expression in CHO: Assessing small molecule mass transfer effects in various reactor configurations. Biotechnol Bioeng, 2017. 114(7): p. 1478-1486.
13. Chappell, J., K. Jensen, and P. S. Freemont, Validation of an entirely in vitro approach for rapid prototyping of DNA regulatory elements for synthetic biology. Nucleic Acids Res, 2013. 41(5): p. 3471-81.
14. Takahashi, M. K., et al., Characterizing and prototyping genetic networks with cell-free transcription-translation reactions. Methods, 2015. 86: p. 60-72.
15. Karim, A. S. and M. C. Jewett, A cell-free framework for rapid biosynthetic pathway prototyping and enzyme discovery. Metab Eng, 2016. 36: p. 116-26.
16. Dudley, Q. M., K. C. Anderson, and M. C. Jewett, Cell-Free Mixing of Escherichia coli Crude Extracts to Prototype and Rationally Engineer High-Titer Mevalonate Synthesis. ACS Synth Biol, 2016. 5(12): p. 1578-1588.
17. Watanabe, M., et al., Cell-free protein synthesis for structure determination by X-ray crystallography. Methods Mol Biol, 2010. 607: p. 149-60.
18. Martemyanov, K. A., et al., Cell-free production of biologically active polypeptides: application to the synthesis of antibacterial peptide cecropin. Protein Expr Purif, 2001. 21(3): p. 456-61.
19. Renesto, P. and D. Raoult, From genes to proteins: in vitro expression of rickettsial proteins. Ann N Y Acad Sci, 2003. 990: p. 642-52.
20. Xu, Z., et al., High-level expression of soluble human beta-defensin-2 fused with green fluorescent protein in Escherichia coli cell-free system. Appl Biochem Biotechnol, 2005. 127(1): p. 53-62.
21. Sullivan, C. J., et al., A cell-free expression and purification process for rapid production of protein biologics. Biotechnol J, 2016. 11(2): p. 238-48.
22. Li, J., et al., Cell-free protein synthesis enables high yielding synthesis of an active multicopper oxidase. Biotechnol J, 2016. 11(2): p. 212-8.
23. Heinzelman, P., J. A. Schoborg, and M. C. Jewett, pH responsive granulocyte colony-stimulating factor variants with implications for treating Alzheimer's disease and other central nervous system disorders. Protein Eng Des Sel, 2015. 28(10): p. 481-9.
24. Kightlinger, W., et al., Design of glycosylation sites by rapid synthesis and analysis of glycosyltransferases. Nat Chem Biol, 2018.
25. Schoborg, J. A., et al., A cell-free platform for rapid synthesis and testing of active oligosaccharyltransferases. Biotechnol Bioeng, 2018. 115(3): p. 739-750.
26. Pardee, K., et al., Rapid, Low-Cost Detection of Zika Virus Using Programmable Biomolecular Components. Cell, 2016. 165(5): p. 1255-1266.
27. Slomovic, S., K. Pardee, and J. J. Collins, Synthetic biology devices for in vitro and in vivo diagnostics. Proc Natl Acad Sci USA, 2015. 112(47): p. 14429-35.
28. Gootenberg, J. S., et al., Nucleic acid detection with CRISPR-Cas13a/C2c2. Science, 2017. 356(6336): p. 438-442.
29. Pardee, K., et al., Portable, On-Demand Biomolecular Manufacturing. Cell, 2016. 167(1): p. 248-259 e12.
30. Karig, D. K., et al., Preservation of protein expression systems at elevated temperatures for portable therapeutic production. J R Soc Interface, 2017. 14(129).
31. Smith, M. T., et al., Lyophilized Escherichia coli-based cell-free systems for robust, high-density, long-term storage. Biotechniques, 2014. 56(4): p. 186-93.
32. Hunt, J. P., et al., The growing impact of lyophilized cell-free protein expression systems. Bioengineered, 2017. 8(4): p. 325-330.
33. Hodgman, C. E. and M. C. Jewett, Optimized extract preparation methods and reaction conditions for improved yeast cell-free protein synthesis. Biotechnol Bioeng, 2013. 110(10): p. 2643-54.
34. Zemella, A., et al., Cell-Free Protein Synthesis: Pros and Cons of Prokaryotic and Eukaryotic Systems. Chembiochem, 2015. 16(17): p. 2420-31.
35. Li, J., et al., Establishing a high yielding streptomyces-based cell-free protein synthesis system. Biotechnol Bioeng, 2017. 114(6): p. 1343-1353.
36. Li, J., H. Wang, and M. C. Jewett, Expanding the palette of Streptomyces-based cell-free protein synthesis systems with enhanced yields. Biochemical Engineering Journal, 2018. 130: p. 29-33.
37. Moore, S. J., et al., Streptomyces venezuelae TX-TL—a next generation cell-free synthetic biology tool. Biotechnol J, 2017. 12(4).
38. Kelwick, R., et al., Development of a Bacillus subtilis cell-free transcription-translation system for prototyping regulatory elements. Metab Eng, 2016. 38: p. 370-381.
39. Moore, S. J., et al., Rapid acquisition and model-based analysis of cell-free transcription-translation reactions from nonmodel bacteria. Proc Natl Acad Sci USA, 2018. 115(19): p. E4340-E4349.
40. Payne, W. J., Studies on bacterial utilization of uronic acids. III. Induction of oxidative enzymes in a marine isolate. J Bacteriol, 1958. 76(3): p. 301-7.
41. Eagon, R. G., Pseudomonas natriegens, a marine bacterium with a generation time of less than 10 minutes. J Bacteriol, 1962. 83: p. 736-7.
42. Weinstock, M. T., et al., Vibrio natriegens as a fast-growing host for molecular biology. Nat Methods, 2016. 13(10): p. 849-51.
43. Lee, H. H., et al., Vibrio natriegens, a new genomic powerhouse. bioRxiv, 2016. 058487.
44. Hirose, T. and Sugiura, M. Cis-acting elements and trans-acting factors for accurate translation of chloroplast psbA nRNAs: development of an in vitro translation system from tobacco chloroplasts. EMBO J. 15, 1687-1695. doi: 10.1002/j.1460-2075.1996.tb00514.x (1996).
45. Yukawa M., Kuroda H., and Sugiura, M., A new in vitro translation system for non-radioactive assay from tobacco chloroplasts: effect of pre-mRNA processing on translation in vitro. Yukawa M., Kuroda H., and Sugiura M., Plant J., 49, 367-376, doi: 10.1111/j.1365-313X.2006.02948.x (2007)
46. Buntru M., Vogel S., Spiegel H. and Stefan Schillberg S. Tobacco BY-2 cell-free lysate: an alternative and highly-productive plant-based in vitro translation system. BMC Biotechnology 14:37, doi: 10.1186/1472-6750-14-37 (2014).

### PATENT DOCUMENTS

U.S. Pat. Nos. 5,478,730; 5,556,769; 5,665,563; 6,168,931; 6,518,058; 6,783,957; 6,869,774; 6,994,986; 7,118,883; 7,189,528; 7,312,049; 7,338,789; 7,387,884; 7,399,610; 8,357,529; 8,574,880; 8,703,471; 8,999,668; 9,410,170; and US952813; the contents of which are incorporated herein by reference in their entirety.

U.S. Patent Publications: US20020058303; US2002034559; US20040191858; US20040209321; US20050032086; US20050064592; US20050148046; US20050170452; US20050186655; US20060211085; US20060234345; US20060252672; US20060257399; US20060286637; US20070026485; US20070154983; US20070141661; US20070178551; US20080138857; US20080221311; US20080248521; US20090042244; US20120088269; US20140295492; US20160060301; US20180016612; US20180016614; US20160312312; and US20160362708; the contents of which are incorporated herein by reference in their entirety.

Published International Applications: WO2003056914A1; WO2004013151A2; WO2004035605A2; WO2006102652A2; WO2006119987A2; WO2007120932A2; W2014144583; and W2017117539; the contents of which are incorporated herein by reference in their entirety.

In the foregoing description, it will be readily apparent to one skilled in the art that varying substitutions and modifications may be made to the invention disclosed herein without departing from the scope and spirit of the invention. The invention illustratively described herein suitably may be practiced in the absence of any element or elements, limitation or limitations which is not specifically disclosed herein. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention. Thus, it should be understood that although the present invention has been illustrated by specific embodiments and optional features, modification and/or variation of the concepts herein disclosed may be resorted to by those skilled in the art, and that such modifications and variations are considered to be within the scope of this invention.

All methods described herein can be performed in any suitable order unless otherwise indicated herein or otherwise clearly contradicted by context. The use of any and all examples provided herein, is intended merely to better illuminate the invention and does not pose a limitation on the scope of the invention unless otherwise claimed. No language in the specification should be construed as indicating any non-claimed element as essential to the practice of the invention.

Citations to a number of patent and non-patent references are made herein. The cited references are incorporated by reference herein in their entireties. In the event that there is an inconsistency between a definition of a term in the specification as compared to a definition of the term in a cited reference, the term should be interpreted based on the definition in the specification.

We claim:

1. A cell-free protein synthesis platform for in vitro transcription of mRNA and/or in vitro translation of polypeptides, the platform comprising as a component:
(a) a plastid extract prepared from isolated intact plastids by lysing the isolated intact plastids and separating a plastid extract from the lysed plastids;
and the platform optionally comprising one or more of the following components:
(b) a reaction buffer;
(c) an RNA polymerase; and
(d) the transcription template,
wherein the RNA polymerase is capable of transcribing the transcription template to form a translation template and the plastid extract can sustain protein synthesis through a combined transcription/translation reaction.

2. The platform of claim 1, wherein the plastid extract is prepared from isolated intact chloroplasts.

3. The platform of claim 1, wherein one or more of the following conditions are met: (i) the plant is grown for about 2-8 weeks prior to preparing the plastid extract; (ii) the plant is grown in about 6 hours dark and in about 18 hours light during the 2-8 weeks; and (iii) the plant is incubated in total dark for 10-12 hours before preparing the plastid extract.

4. The platform of claim 1, wherein the plastid extract is prepared from a species selected from Nicotiana spp.

5. The platform of claim 1, wherein the plastid extract is prepared from a species selected from Maize spp.

6. The platform of claim 1, wherein the plastid extract is prepared from a plant that is engineered to be deficient in a negative effector for in vitro transcription and or a negative effector for in vitro translation.

7. The platform of claim 1, wherein the plastid extract is prepared from a plant that has been engineered to express T7 RNA polymerase in the chloroplast or in the nucleus and T7 RNA polymerase is targeted to the chloroplast.

8. The platform of claim 1, wherein the platform or any component of the platform is freeze-dried.

9. The platform of claim 1, wherein the plastid extract is prepared by a method that includes a step of adding glycerol at a concentration of about 5-15% to the intact plastids prior to lysing the intact plastids

10. A method for in vitro transcription of mRNA and/or in vitro translation of mRNA to prepare a polypeptide, the method comprising reacting a reaction mixture comprising:
(a) the cell-free protein synthesis platform of claim 1;
(b) a template for transcription of the mRNA and/or a template for translation to prepare the polypeptide;
(c) NTP's for synthesis of the mRNA at a concentration of 0.5 to 4 mM for synthesis of the mRNA and/or amino acids at a concentration of 0.5 to 4 mM for synthesis of the polypeptide;
(d) co-factors, enzymes and/or other reagents necessary for the transcription and/or translation; and
(e) magnesium at a concentration of from about 3 mM to about 20 mM.

11. The method of claim 10, wherein the reaction mixture further comprises one or more of: (i) potassium at a concentration of about 2-200 mM; (ii) salts at a total concentration of between about 50-400 mM.

12. The method of claim 10, wherein the reaction mixture further comprises an energy regeneration system that comprises creatine kinase and creatine phosphate.

13. The method of claim 10, wherein the template for the mRNA includes modifications in the 5′UTR or modification in the 3′UTR which modifications facilitate efficient transcription and/or translation.

14. The method of claim 10, wherein the reaction mixture further comprises a DNA-dependent RNA polymerase.

15. The method of claim 10, wherein the reaction mixture or any component of the reaction mixture has been freeze-dried and rehydrated prior to use in the method.

16. A method comprising:
(a) creating a test library of genetic parts or components of plants; and
(b) testing the function of the genetic parts or components of the test library in the cell-free protein synthesis platform of claim 1; and
(c) optionally assessing gene expression, and optionally using information obtained from assessing gene expression to modify gene expression in a plant; and
(d) optionally wherein the method is used to characterize and assess the genetic parts or components and modulate gene expression prior to studying the genetic parts and gene expression in plants.

17. A method comprising:
(a) creating a test library of codon-optimized constructs of a gene product; and
(b) testing expression of the codon-optimized constructs of the test library in the cell-free protein synthesis platform of claim 1; and
(c) optionally using information obtained from testing expression of the codon-optimized constructs to modify expression in a plant; and
(d) optionally wherein the method is used to characterize and assess codon optimization and modulate gene expression prior to studying codon optimization and fine-tuning of gene expression in plants.

18. A method comprising:
(a) creating a test library of constructs expressing several genes; and
(b) testing expression of the constructs of the test library in the cell-free protein synthesis platform of claim 1; and
(c) optionally using information obtained from testing expression of the construct to modify gene expression in a plant; and
(d) optionally wherein the method is used to is used to provide information to a geneticist regarding the design of multi-gene functions.

19. A method comprising:
(a) creating a test library of constructs of a biosynthetic pathway expressing enzymes; and
(b) testing the constructs of the test library in the cell-free protein synthesis platform of claim 1; and.
(c) assessing gene expression, metabolite production, requirement of accessory proteins or other molecules, and/or metabolic parameters;
(d) optionally wherein the method is used to aid design of multi-gene functions; and
(e) optionally wherein the method is used to aid the design and function of enzyme pathways for example in order to modulate expression levels of the enzymes and testing possible accessory proteins or other molecules required for proper pathway function.

20. A method for preparing an extract from plastids of a plant, the method comprising:
(a) lysing plant cells from a plant that has been grown for about 2-8 weeks;
(b) isolating intact plastids from the lysed plant cells;
(c) adding glycerol to the isolated intact plastids at a concentration of about 5-15%; and
(d) lysing the intact plastids and separating the extract from the lysed plastids.
